(19) United States
(12) Patent Application Publication
(10) Pub. No.: US 2015/0030582 A1
(43) Pub. Date: Jan. 29, 2015

(54) Lysosomal Storage Disease Enzyme

(71) Applicants: Alex J. Harvey, Athens, GA (US); Anthony Quinn, Gloucester, MA (US)

(72) Inventors: Alex J. Harvey, Athens, GA (US); Anthony Quinn, Gloucester, MA (US)

(73) Assignee: SYNAGEVA BIOPHARMA CORP., Lexington, MA (US)

(21) Appl. No.: 14/491,604

(22) Filed: Sep. 19, 2014

Related U.S. Application Data
(63) Continuation-in-part of application No. 13/642,790, filed on Apr. 30, 2013.
(60) Provisional application No. 61/343,177, filed on Apr. 23, 2010.
(60) Provisional application No. 61/396,376, filed on May 26, 2010.
(60) Provisional application No. 61/403,011, filed on Sep. 9, 2010.
(60) Provisional application No. 61/456,014, filed on Oct. 29, 2010.
(60) Provisional application No. 61/432,372, filed on Jan. 13, 2011.

(51) Int. Cl. A61K 38/46

(52) U.S. Cl. CPC A61K 38/465; C12Y 301/01013
USPC 424/94.6

(57) ABSTRACT

The present invention provides compositions of recombinant human lysosomal acid lipase having particular glycosylation patterns for internalization into target cells, a vector containing the nucleic acid encoding human lysosomal acid lipase, a host cell transformed with the vector, pharmaceutical compositions comprising the recombinant human lysosomal acid lipase and method of treating conditions associated with lysosomal acid lipase deficiency.

### RELATED APPLICATIONS

This application is a Continuation-in-part application of U.S. patent application Ser. No. 13/642,790, filed Apr. 30, 2013, which is a national stage entry of PCT/US2011/033699, international filing date of Apr. 23, 2011, which claims the benefit of U.S. Provisional Application No. 61/343,177, filed on Apr. 23, 2010, U.S. Provisional Application No. 61/396,376, filed on May 26, 2010, U.S. Provisional Application No. 61/403,011, filed on Sep. 9, 2010, U.S. Provisional Application No. 61/456,014, filed on Oct. 29, 2010, U.S. Provisional Application No. 61/432,372, filed on Jan. 13, 2011. The entire teachings of the above applications are incorporated herein by reference.

### BACKGROUND OF THE INVENTION

Lysosomal Acid Lipase (LAL) deficiency is a very rare lysosomal storage disease (LSD) characterized by a failure to breakdown cholesteryl esters (CE) and triglycerides (TAG) in lysosomes due to a deficiency of the enzyme. LAL deficiency resembles other lysosomal storage disorders with the accumulation of substrate in a number of tissues and cell types. In LAL deficiency substrate accumulation is most marked in cells of the reticuloendothelial system including Kupffer cells in the liver, histiocytes in the spleen and in the lamina propria of the small intestine. Reticuloendothelial cells express the macrophage mannose/N-acetylglucosamine receptor (also known as macrophage mannose receptor or MMR, CD206), which mediates binding, cell uptake and lysosomal internalization of proteins with GlcNAc or mannose terminated N-glycans, and provides a pathway for the potential correction of the enzyme deficiency in these key cell types.

LAL deficiency is a multi-system disease that most commonly manifests with gastrointestinal, liver and cardiovascular complications and is associated with significant morbidity and mortality. The clinical effects of LAL deficiency are due to a massive accumulation of lipid material in the lysosomes in a number of tissues and a profound disturbance in cholesterol and lipid homeostatic mechanisms, including substantial increases in hepatic cholesterol synthesis. LAL deficiency presents of at least two phenotypes, Wolman Disease (WD) and Cholesteryl Ester Storage Disease (CESD).

Wolman Disease is the most aggressive presentation of LAL deficiency. This phenotype is characterized by gastrointestinal and hepatic manifestations including growth failure, malabsorption, steatorrhea, profound weight loss and hepatomegaly. Wolman Disease is rapidly progressive and fatal usually within the first year of life. Case report review indicates survival beyond 12 months of age is highly unusual for patients who present with growth failure due to LAL deficiency in the first year of life. In this most aggressive form, growth failure is the predominant clinical feature and is a key contributor to the early mortality. Hepatic involvement as evidenced by liver enlargement and elevation of transaminases is also common in infants. Physical findings include abdominal distention with hepatomegaly and splenomegaly, and radiographic examination often reveals calcification of the adrenal glands. Laboratory evaluations typically reveal elevated levels of serum transaminases and absent or markedly reduced LAL enzyme activity. Elevated blood levels of cholesterol and triglycerides are also seen in patients.

Current treatment options for Wolman Disease are extremely limited. Antibiotics are administered to infants with pyrexia and/or evidence of infection. Steroid replacement therapy for adrenal insufficiency and specialized nutritional support may be prescribed and while there is no evidence that these interventions prevents death, it is also unclear at present if they have an impact on short term survival. In a series of four patients with LAL deficiency treated with bone marrow transplantation, all four patients died due to complications of the procedure within months of transplantation.

Patients with LAL deficiency can also present later in life with predominant liver and cardiovascular involvement and this is often called Cholesteryl Ester Storage Disease (CESD). In CESD, the liver is severely affected with marked hepatomegaly, hepatocyte necrosis, elevation of transaminases, cirrhosis and fibrosis. Due to the increased levels of CE and TG, hyperlipidemia and accelerated atherosclerosis are also seen in LAL deficiency. Particularly, an accumulation of fatty deposits on the artery walls is described early in life. The deposits narrow the arterial lumen and can lead to vessel occlusion increasing the risk of significant cardiovascular events including myocardial infarction and strokes. The presentation of CESD is highly variable with some patients going undiagnosed until complications manifest in late adulthood, while others can have liver dysfunction presenting in early childhood. CESD is associated with shortened lifespan and significant ill health; the life expectancy of those with CESD depends on the severity of the associated complications.

Current treatment options for the CESD phenotype are focused on controlling lipid accumulation through diet that excludes foods rich in cholesterol and triglycerides and suppression of cholesterol synthesis and apolipoprotein B production through administration of cholesterol lowering drugs. Although some clinical improvement may be seen, the underlying disease manifestations persist and disease progression still occurs.

In most cases, therapy for LAL deficiencies requires life-long treatment. In addition, due to the high cost of protein therapeutics, it is desirable to administer a minimum effective amount of therapeutic to treat LAL deficiency. However, to date, there is no effective therapy for treating LAL deficiency, particularly the patients suffering from Wolman Disease and CESD. Therefore, there is a strong need for an effective therapy with a minimized frequency of administration in order to improve the quality of life for patients. There is also a need for a high expressing and robust protein production platform which can produce LAL proteins that are stable and efficiently targeted to the lysosomal compartment in the affected tissue cells in patients.

### SUMMARY OF THE INVENTION

Disclosed herein are compositions of LAL which are particularly suited for use in therapy, for example, for treatment of conditions associated with LAL deficiency. The LAL molecules described herein contain particular glycan structures which afford efficient and rapid uptake into lysosomes of cells when administered into a subject, for example, a human subject.

In one aspect, the compositions disclosed herein comprise human LAL wherein a substantial percentage of the human LAL contain at least one mannose-6-phosphate glycan moiety, which can serve as a ligand for internalization by the mannose-6-phosphate receptor on the surface of cells found, for example, on hepatocytes. In one embodiment, 30% or more, for example, at least 30%, at least 40%, at least 50%, at least 60%, at least 70%, at least 80%, at least 90%, at least 95%, at least 97%, or at least 99%, of the LAL contained in the composition contains at least one mannose-6-phosphate moiety. The mannose-6-phosphate moiety can be found, for example, on an N-glycan structure located at one or more residues selected from the group consisting of Asn¹⁵, Asn⁵¹, Asn⁸⁰, Asn¹⁴⁰, Asn²⁵² and Asn³⁰⁰ of SEQ ID NO:2.

In another aspect, the compositions disclosed herein comprise human LAL wherein a substantial percentage of the human LAL does not contain a sialic acid moiety in any of its N-glycan structures, which can sometimes interfere with internalization of the enzyme into cells. In one embodiment, 15% or less, for example, 10% or less, 5% or less, 2% or less, 1% or less, or essentially none, of the LAL contained in the composition contains a sialic acid moiety in any of its N-glycan structures.

In another aspect, the compositions disclosed herein comprise human LAL wherein a substantial percentage of the human LAL does not contain a fucose moiety in any of its N-glycan structures. In one embodiment, 50% or less, for example, 50% or less, 40% or less, 30% or less, 20% or less, 10% or less, 5% or less, 2% or less, 1% or less, or essentially none, of the LAL contained in the composition contains a fucose moiety in any of its N-glycan structures.

In yet another aspect, vectors, host cells, expression systems and associated methods suitable for producing the LAL-containing compositions are described.

Typically, the LAL of the invention discussed and disclosed herein is human LAL. In one embodiment, the composition comprising LAL includes the mature LAL having the amino acid sequence of:

(SEQ ID NO: 2)
SGGKLTAVDPETNMNVSEIISYWGFPSEEYLVETEDGYILCLNRIPHGRK
NHSDKGPKPVVFLQHGLLADSSNWVTNLANSSLGFILADAGFDVWMGNSR
GNTWSRKHKTLSVSQDEFWAFSYDEMAKYDLPASINFILNKTGQEQVYYV
GHSQGTTIGFIAFSQIPELAKRIKMFFALGPVASVAFCTSPMAKLGRLPD
HLIKDLFGDKEFLPQSAFLKWLGTHVCTHVILKELCGNLCFLLCGFNERN
LNMSRVDVYTTHSPAGTSVQNMLHWSQAVKFQKFQAFDWGSSAKNYFHYN
QSYPPTYNVKDMLVPTAVWSGGHDWLADVYDVNILLTQITNLVFHESIPE
WEHLDFIWGLDAPWRLYNKIINLMRKYQ.

In another embodiment, the mature LAL has the amino acid sequence of:

(SEQ ID NO: 3)
GKLTAVDPETNMNVSEIISYWGFPSEEYLVETEDGYILCLNRIPHGRKNH
SDKGPKPVVFLQHGLLADSSNWVTNLANSSLGFILADAGFDVWMGNSRGN
TWSRKHKTLSVSQDEFWAFSYDEMAKYDLPASINFILNKTGQEQVYYVGH
SQGTTIGFIAFSQIPELAKRIKMFFALGPVASVAFCTSPMAKLGRLPDHL
IKDLFGDKEFLPQSAFLKWLGTHVCTHVILKELCGNLCFLLCGFNERNLN
MSRVDVYTTHSPAGTSVQNMLHWSQAVKFQKFQAFDWGSSAKNYFHYNQS
YPPTYNVKDMLVPTAVWSGGHDWLADVYDVNILLTQITNLVFHESIPEWE
HLDFIWGLDAPWRLYNKIINLMRKYQ.

In another embodiment, the mature LAL has the amino acid sequence of:

(SEQ ID NO: 4)
TAVDPETNMNVSEIISYWGFPSEEYLVETEDGYILCLNRIPHGRKNHSDK
GPKPVVFLQHGLLADSSNWVTNLANSSLGFILADAGFDVWMGNSRGNTWS
RKHKTLSVSQDEFWAFSYDEMAKYDLPASINFILNKTGQEQVYYVGHSQG
TTIGFIAFSQIPELAKRIKMFEALGPVASVAFCTSPMAKLGRLPDHLIKD
LFGDKEFLPQSAFLKWLGTHVCTHVILKELCGNLCFLLCGFNERNLNMSR
VDVYTTHSPAGTSVQNMLHWSQAVKFQKFQAFDWGSSAKNYFHINQSYPP
TYNVKDMINPTAVWSGGHDWLADVYDVNILLTQITNLVFHESIPEWEHLD
FIWGLDAPWRLYNKIINLMRKYQ.

In another embodiment, the mature LAL has the amino acid sequence of:

(SEQ ID NO: 19)
AVDPETNMNVSEIISYWGFPSEEYLVETEDGYILCLNRIPHGRKNHSDKG
PKPVVFLQHGLLADSSNWVTNLANSSLGFILADAGFDVWMGNSRGNTWSR
KHKTLSVSQDEFWAFSYDEMAKYDLPASINFILNKTGQEQVYYVGHSQGT
TIGFIAFSQIPELAKRIKMFFALGPVASVAFCTSPMAKLGRLPDHLIKDL
FGDKEFLPQSAFLKWLGTHVCTHVILKELCGNLCFLLCGFNERNLNMSRV
DVYTTHSPAGTSVQNMLHWSQAVKFQKFQAFDWGSSAKNYFHYNQSYPPT
YNVKDMLVPTAVWSGGHDWLADVYDVNILLTQITNLVFHESIPEWEHLDF
IWGLDAPWRLYNKIINLMRKYQ.

In another embodiment, the mature LAL is a mixture of at least two polypeptides selected from the group consisting of: SEQ ID NO:2, SEQ ID NO:3, SEQ ID NO:4, and SEQ ID NO:19.

The invention also provides for compositions which contain isolated mixtures of an individual type of useful protein molecule, such as those proteins disclosed herein, where one or more of the protein molecules contained in the mixture has a specific oligosaccharide structure attached, in particular an oligosaccharide structure disclosed herein. For example, the invention provides for isolated mixtures of LAL molecules, for example, human LAL molecules which contain an LAL molecule glycosylated with one or more of the following structures A-n to O-n:

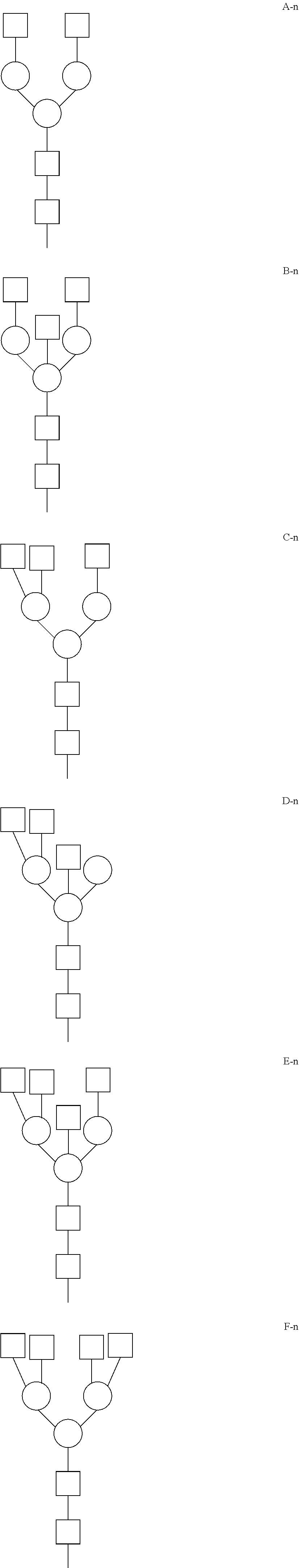

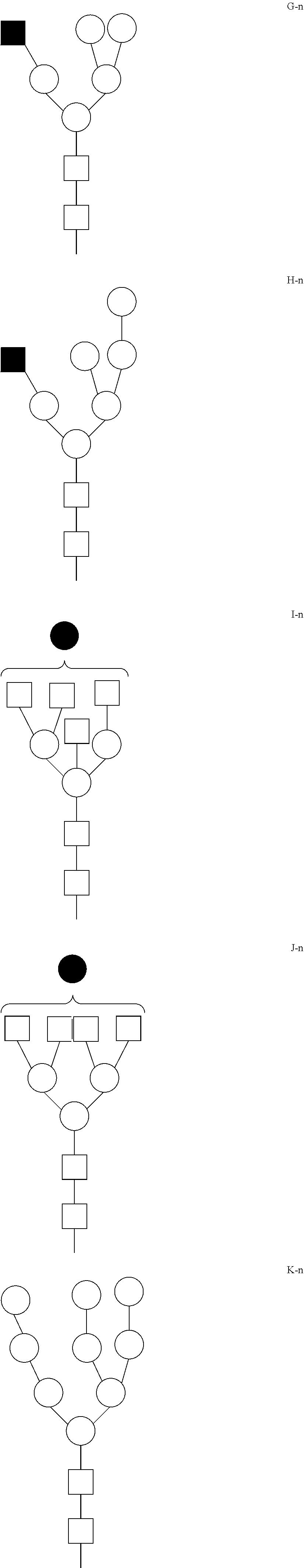

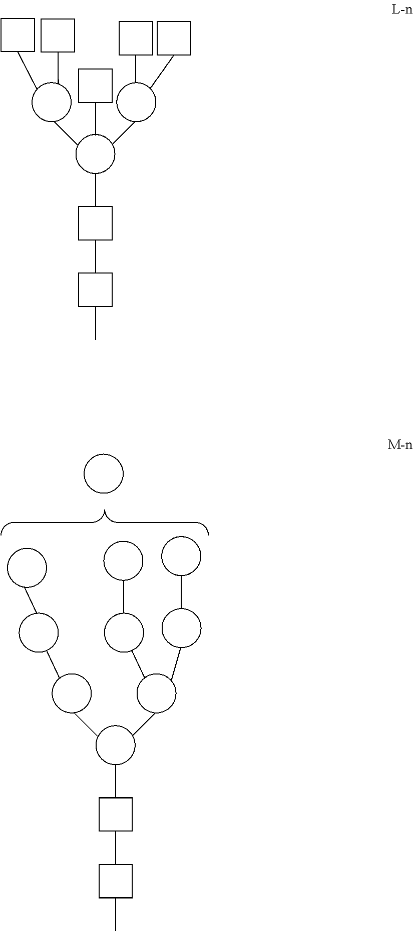

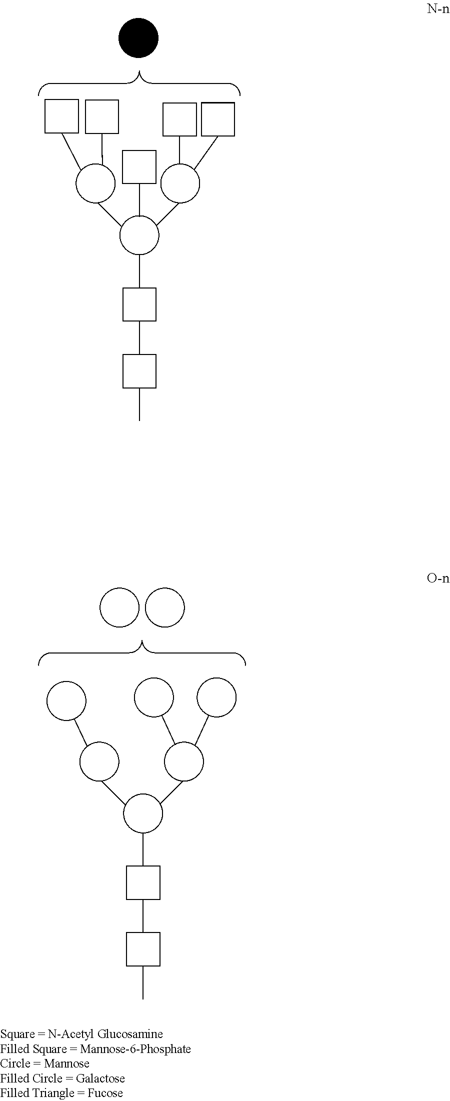

According to one aspect of the present invention, a composition comprises any isolated individual or combination of the polypeptides described above. In one embodiment, the composition can be a pharmaceutical composition, for example, a formulation that further comprises pharmaceutically acceptable carriers, such that the composition is, for example, suitable for administration into a subject (e.g., a human, particularly a patient suffering from or diagnosed with a condition). The composition can be administered any number of ways, including by intravenous administration. In another embodiment, the composition can further comprise a second agent. Such an agent can be a medicament, or an agent which can influence or modify a biological process when administered into a subject. For example, the second agent can be an immunomodulatory agent. Such immunomodulatory agents can include any agent which, when administered together (i.e., administered at the same time as, or shortly before or after) with any of the LAL compositions described herein, may have the effect of reducing the immunogenicity of the LAL composition in the subject (e.g., Rituximab, or any other B-cell depleting antibody).

In a final aspect, methods and compositions for the treatment of symptoms associated with LAL deficiency are disclosed.

Additional objects and aspects of the present invention will become more apparent upon review of the detailed description set forth below when taken in conjunction with the accompanying figures and sequences.

### BRIEF DESCRIPTION OF THE DRAWINGS

FIG. 1 depicts the amino acid sequences of human LAL. The amino acid sequence of the recombinant hLAL shows 100% homologous to that of natural human LAL. The mature form of hLAL is underlined.

FIG. 2 depicts the nucleotide sequence of recombinant hLAL, the rhLAL transgene of pALVIN-OVR1-I-hLAL-dSA.

FIGS. 3A and 3B depict diagrams of pALVIN-OVR1-I-hLAL-dSA and its proviral region. FIG. 3A depicts a diagram of human LAL retrovirus expression vector used in the production of transducing particles is diagrammed (the DNA sequence of the plasmid is located in Appendix A). FIG. 3A depicts pALVIN-OVR1-I-hLAL-dSA proviral region that has been integrated into the genome. SIN LTR, self-inactivating long terminal repeat; OV DHSIII enhancer, DNase hypersensitive site III of the ovalbumin gene; OV Intron; ovalbumin 5′ untranslated region and intron 1; hLAL, human LAL cDNA; OV 3′ UTR, ovalbumin gene 3′ untranslated region; partial gag, partial gag gene; LTR, long terminal repeat.

FIG. 4 depicts a nucleotide sequence of pALVIN-OVR1-I-hLAL-dSA.

FIG. 5 depicts a nucleotide sequence of pALVIN-OVR1-I-hLAL-dSA proviral region that has been integrated into the genome.

FIG. 6 depicts a nucleotide sequence of pALVIN-OV-1.1-I vector.

FIG. 7 depicts a nucleotide sequence of rhLAL adaptor.

FIG. 8 depicts a nucleotide sequence of rhLAL including the partial ovalbumin promoter.

FIG. 9 depicts a nucleotide sequence of OVR1 promoter.

FIG. 10 depicts schematics of the steps used to construct the pALVIN-OVR1-I-hLAL-dSA vector,

FIG. 11 depicts a real-time PCR analysis of blood DNA samples from a hemizygous transgenic G1 offspring of XLL109. The signals from duplicate DNA samples of hemizygous G1 progeny, 1LL7466, are indicated by the curves that initiate an increase in Delta Rn before cycle 22. The curves for two non-transgenic progeny are shown; these curves stay at or near baseline through at least 34 cycles.

FIGS. 12A-D depict Southern analysis of G1 chickens carrying the ALVIN-OVR1-I-hLAL-dSA transgene. FIG. 12A illustrates schematic of the integrated transgene and flanking genomic regions is shown with the known position of the transgene BlpI site and predicted position of the flanking genomic BlpI sites. The position of the OV promoter probe and the hLAL coding sequence probe (hLAL probe) are indicated by the black bars. The positions of the 4.3 kb and 10.6 kb bands detected in the Southern analysis are shown as well as the predicted sizes of the genomic and transgene portions of the 4.3 kb and 10.6 kb bands. FIG. 12B illustrates a Southern blot of genomic DNA digested with BlpI and probed with the OV probe. WT CTRL is genomic DNA isolated from a non-transgenic chicken. The ID numbers of the G1 transgenics are indicated above the lanes. The position and size (kb) of the molecular weight markers are shown to the left of the blot. The position and size of the detected transgene fragment (4.3 kb) and endogenous ovalbumin gene (4.1 kb) are shown to the right of the blot. FIG. 12C depicts a Southern blot was probed with the hLAL probe. The position and size of the detected transgene fragment (10.6 kb) is shown to the right of the blot. FIG. 12D depicts a section of the image shown in FIG. 12B at a larger scale to demonstrate the presence of the 4.1 and 4.3 kb bands.

FIG. 13A depicts schematic of the ALVIN-OVR1-I-hLAL-dSA transgene. The size of ApaLI bands predicted to be detected by the OV probe and hLAL probe are also shown. FIG. 13B depicts schematic of a Southern blot analysis of the ALVIN-OVR1-I-hLAL-dSA transgene for confirmation of transgene size. Southern blot of genomic DNA digested with ApaLI and probed with either the OV probe (left panel) or hLAL probe (right panel). WT CTRL is genomic DNA isolated from a non-transgenic chicken. The ID number of the G1s is indicated above each lane. The position and size (kb) of the molecular weight markers are shown to the left of the blots. The position and size of the detected transgene fragments (OV promoter probe, 3.6 kb; hLAL probe, 3.8 kb) and endogenous ovalbumin gene (7.7 kb) are shown to the right of the blots.

FIG. 14 depicts a lineage of transgenic chickens. Shown for each chicken are the generation number (G0, G1 or G2), identification number, gender and hatch date. Other G1 chickens are those of other lineages.

FIG. 15 depicts the purification steps of hLAL from egg white.

FIG. 16 depicts N-glycans found as an N-linked Glycosylation structure in LAL produced in accordance with the invention. Square, N-Acetyl glucosamine; Filled square, mannose-6-phosphate; circle, mannose; filled circle; galactose; and filled triangle, fucose.

FIG. 17 depicts the relative position of predicted N-glycan sites indicated on the LAL polypeptide (arrow) set forth in SEQ ID NO:1. N-glycans that are structurally representative of those detected at each site are shown. Square, N-Acetyl glucosamine; Filled square, mannose-6-phosphate; circle, mannose; filled circle; galactose; and filled triangle, fucose.

FIG. 18 depicts phosphorylated N-glycans released by PNGase and analyzed by MALDI-TOF. Structures are shown.

FIG. 19 depicts the effect of dephosphorylation of LAL on HPAEC-PAD retention time of N-glycans. LAL produced in accordance with the invention was dephosphorylated with bacterial alkaline phosphatase (upper panel) or left untreated (lower panel). Released N-glycans were analyzed by HPAEC-PAD.

FIG. 20 depicts the co-localization of recombinant human LAL (SBC-102) and lysosomal marker in the lysosomes of these cells examined by confocal fluorescence microscopy using a sequential scanning mode.

FIG. 21 depicts the binding specificity of recombinant human LAL (SBC-102) to the GlcNAc/mannose receptor assessed by competitive binding assays using the macrophage cell line, NR8383.

FIG. 22 depicts the activity of recombinant human LAL in cells in normal and LAL-deficient cells in vitro.

FIG. 23 depicts the effect of recombinant human LAL (SBC-102) treatment on internal organs mass of LAL deficient rats. Organ size is represented as percent of body weight determined at 8 weeks of age, in LAL^{-/-} rats and LAL^{+/+} rats after weekly administration of vehicle or SBC-102 at 5 mg/kg for 4 weeks.

FIG. 24 depicts body weight in wild type and LAL-deficient rats after weekly administration of vehicle or SBC-102 at 5 mg·kg⁻¹ for 4 weeks. Dose administration is highlighted on X-axis by diamonds starting at 4 week.

FIG. 25 shows liver cholesterol, cholesteryl ester and triglyceride levels determined at 8 weeks of age in WT and LAL deficient rats after weekly administration of vehicle or recombinant human LAL (SBC-102) at 5 mg·kg⁻¹ for 4 weeks.

FIG. 26 depicts percent increase in body weight in LAL-deficient rats after 4 weeks administration recombinant human LAL (SBC-102) at the indicated levels and schedules, determined at 8 weeks of age.

FIG. 27 shows liver weight, as a percent of body weight, in LAL-deficient rats after 4 weeks administration SBC-102 at the indicated levels and schedules, determined at 8 weeks of age.

FIG. 28 shows tissue cholesteryl ester levels in LAL-deficient rats after 4 weeks administration SBC-102 at the indicated levels and schedules, determined at 8 weeks of age.

FIG. 29 shows the daily progress in weight gain of rats which were administered either 1 mg/kg of LAL per week or 5 mg/kg of LAL per week or 5 mg/kg of LAL per two weeks.

FIG. 30 depicts the gross pathological examination of treated animals showing a substantial normalization in liver size and color as can be seen in the dissection at the top panels and histopathology of liver tissue from LAL of treated rats showing normal liver histology in marked contrast to the substantial accumulation of foamy macrophages in the placebo-treated animals at the bottom panels.

### DETAILED DESCRIPTION OF THE INVENTION

### Definitions

Certain definitions are set forth herein to illustrate and define the meaning and scope of the various terms used to describe the invention herein.

As used herein, the term “acceptable” with respect to a formulation, composition or ingredient, as used herein, means having no persistent detrimental effect on the general health of the subject being treated.

As used herein, the term “administration” or “administering” refers to providing a recombinant human lysosomal acid lipase of the invention to a subject in need of treatment.

A “nucleic acid or polynucleotide sequence” includes, but is not limited to, eukaryotic mRNA, cDNA, genomic DNA, and synthetic DNA and RNA sequences, comprising the natural nucleoside bases adenine, guanine, cytosine, thymidine, and uracil. The term also encompasses sequences having one or more modified bases.

The term “avian” as used herein refers to any species, subspecies or race of organism of the taxonomic class ava, such as, but not limited to chicken, turkey, duck, goose, quail, pheasants, parrots, finches, hawks, crows and ratites including ostrich, emu and cassowary. The term includes the various known strains of Gallus gallus, or chickens, (for example, White Leghorn, Brown Leghorn, Barred-Rock, Sussex, New Hampshire, Rhode Island, Australorp, Minorca, Amrox, California Gray), as well as strains of turkeys, pheasants, quails, duck, ostriches and other poultry commonly bred in commercial quantities. It also includes an individual avian organism in all stages of development, including embryonic and fetal stages.

“Therapeutic proteins” or “pharmaceutical proteins” include an amino acid sequence which in whole or in part makes up a drug.

A “coding sequence” or “open reading frame” refers to a polynucleotide or nucleic acid sequence which can be transcribed and translated (in the case of DNA) or translated (in the case of mRNA) into a polypeptide in vitro or in vivo when placed under the control of appropriate regulatory sequences. The boundaries of the coding sequence are determined by a translation start codon at the 5′ (amino) terminus and a translation stop codon at the 3′ (carboxy) terminus. A transcription termination sequence is usually located 3′ to the coding sequence. A coding sequence may be flanked on the 5′ and/or 3′ ends by untranslated regions.

“Exon” refers to that part of a gene which, when transcribed into a nuclear transcript, is “expressed” in the cytoplasmic mRNA after removal of the introns or intervening sequences by nuclear splicing.

Nucleic acid “control sequences” or “regulatory sequences” refer to promoter sequences, translational start and stop codons, ribosome binding sites, polyadenylation signals, transcription termination sequences, upstream regulatory domains, enhancers, and the like, as necessary and sufficient for the transcription and translation of a given coding sequence in a defined host cell. Examples of control sequences suitable for eukaryotic cells are promoters, polyadenylation signals, and enhancers. All of these control sequences need not be present in a recombinant vector so long as those necessary and sufficient for the transcription and translation of the desired gene are present.

“Operably or operatively linked” refers to the configuration of the coding and control sequences so as to perform the desired function. Thus, control sequences operably linked to a coding sequence are capable of effecting the expression of the coding sequence. A coding sequence is operably linked to or under the control of transcriptional regulatory regions in a cell when DNA polymerase binds the promoter sequence and transcribes the coding sequence into mRNA that can be translated into the encoded protein. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

The terms “heterologous” and “exogenous” as they relate to nucleic acid sequences such as coding sequences and control sequences, denote sequences that are not normally associated with a region of a recombinant construct or with a particular chromosomal locus, and/or are not normally associated with a particular cell. Thus, an “exogenous” region of a nucleic acid construct is an identifiable segment of nucleic acid within or attached to another nucleic acid molecule that is not found in association with the other molecule in nature. For example, an exogenous region of a construct could include a coding sequence flanked by sequences not found in association with the coding sequence in nature. Another example of an exogenous coding sequence is a construct where the coding sequence itself is not found in nature (e.g., synthetic sequences having codons different from the native gene). Similarly, a host cell transformed with a construct or nucleic acid which is not normally present in the host cell would be considered exogenous for purposes of this invention.

As used herein the terms “N-glycan,” “oligosaccharide,” “oligosaccharide structure,” “glycosylation pattern,” “glycosylation profile” and “glycosylation structure” have essentially the same meaning and each refer to one or more structures which are formed from sugar residues and are attached to glycosylated proteins.

“Exogenous protein” as used herein refers to a protein not naturally present in a particular tissue or cell, a protein that is the expression product of an exogenous expression construct or transgene, or a protein not naturally present in a given quantity in a particular tissue or cell. A protein that is exogenous to an egg is a protein that is not normally found in the egg. For example, a protein exogenous to an egg may be a protein that is present in the egg as a result of the expression of a coding sequence present in a transgene of the animal laying the egg.

“Endogenous gene” refers to a naturally occurring gene or fragment thereof normally associated with a particular cell.

“LAL” means “human lysosomal acid lipase,” “SBC-102” or “human lysosomal acid lipase molecule” and these terms are used interchangeably throughout the specification.

The expression products described herein may consist of proteinaceous material having a defined chemical structure. However, the precise structure depends on a number of factors, particularly chemical modifications common to proteins. For example, since all proteins contain ionizable amino and carboxyl groups, the protein may be obtained in acidic or basic salt form, or in neutral form. The primary amino acid sequence may be derivatized using sugar molecules (glycosylation) or by other chemical derivatizations involving covalent or ionic attachment with, for example, lipids, phosphate, acetyl groups and the like, often occurring through association with saccharides. These modifications may occur in vitro or in vivo, the latter being performed by a host cell through post-translational processing systems. Such modifications may increase or decrease the biological activity of the molecule, and such chemically modified molecules are also intended to come within the scope of the invention.

Alternative methods of cloning, amplification, expression, and purification will be apparent to the skilled artisan. Representative methods are disclosed in Sambrook, Fritsch, and Maniatis, Molecular Cloning, a Laboratory Manual, 2nd Ed., Cold Spring Harbor Laboratory (1989).

“Vector” means a polynucleotide comprised of single strand, double strand, circular, or supercoiled DNA or RNA. A typical vector may be comprised of the following elements operatively linked at appropriate distances for allowing functional gene expression: replication origin, promoter, enhancer, 5′ mRNA leader sequence, ribosomal binding site, nucleic acid cassette, termination and polyadenylation sites, and selectable marker sequences. One or more of these elements may be omitted in specific applications. The nucleic acid cassette can include a restriction site for insertion of the nucleic acid sequence to be expressed. In a functional vector the nucleic acid cassette contains the nucleic acid sequence to be expressed including translation initiation and termination sites. An intron optionally may be included in the construct, for example, 5′ to the coding sequence. A vector is constructed so that the particular coding sequence is located in the vector with the appropriate regulatory sequences, the positioning and orientation of the coding sequence with respect to the control sequences being such that the coding sequence is transcribed under the “control” of the control or regulatory sequences. Modification of the sequences encoding the particular protein of interest may be desirable to achieve this end. For example, in some cases it may be necessary to modify the sequence so that it may be attached to the control sequences with the appropriate orientation; or to maintain the reading frame. The control sequences and other regulatory sequences may be ligated to the coding sequence prior to insertion into a vector. Alternatively, the coding sequence can be cloned directly into an expression vector which already contains the control sequences and an appropriate restriction site which is in reading frame with and under regulatory control of the control sequences.

A “promoter” is a site on the DNA to which RNA polymerase binds to initiate transcription of a gene. In some embodiments the promoter can be modified by the addition or deletion of sequences, or replaced with alternative sequences, including natural and synthetic sequences as well as sequences which may be a combination of synthetic and natural sequences. Many eukaryotic promoters contain two types of recognition sequences: the TATA box and the upstream promoter elements. The former, located upstream of the transcription initiation site, is involved in directing RNA polymerase to initiate transcription at the correct site, while the latter appears to determine the rate of transcription and is upstream of the TATA box. Enhancer elements can also stimulate transcription from linked promoters, but many function exclusively in a particular cell type. Many enhancer/promoter elements derived from viruses, e.g., the SV40 promoter, the cytomegalovirus (CMV) promoter, the rous-sarcoma virus (RSV) promoter, and the murine leukemia virus (MLV) promoter are all active in a wide array of cell types, and are termed “ubiquitous.” Alternatively, non-constitutive promoters such as the mouse mammary tumor virus (MMTV) promoter may also be used in the present invention. The nucleic acid sequence inserted in the cloning site may have any open reading frame encoding a polypeptide of interest, with the proviso that where the coding sequence encodes a polypeptide of interest, it should lack cryptic splice sites which can block production of appropriate mRNA molecules and/or produce aberrantly spliced or abnormal mRNA molecules.

As used herein, the term “pharmaceutical composition” refers to a mixture of a compound described herein with other chemical components, such as carriers, stabilizers, diluents, dispersing agents, suspending agents, thickening agents, and/or excipients.

The term “poultry derived” or “avian derived” refers to a composition or substance produced by or obtained from poultry. “Poultry” refers to avians that can be kept as livestock, including but not limited to, chickens, duck, turkey, quail and ratites. For example, “poultry derived” may refer to chicken derived, turkey derived and/or quail derived.

A “retroviral particle,” “transducing particle,” or “transduction particle” refers to a replication-defective or replication-competent virus capable of transducing non-viral DNA or RNA into a cell. In one particularly useful embodiment, retroviral particles used to produce transgenic avians in accordance with the invention are made as disclosed in U.S. Pat. No. 7,524,626, issued Apr. 28, 2009, the disclosure of which is incorporated in its entirety herein by reference.

The terms “transformation,” “transduction” and “transfection” all denote the introduction of a polynucleotide into an avian blastodermal cell. “Magnum” is that part of the oviduct between the infundibulum and the isthmus containing tubular gland cells that synthesize and secrete the egg white proteins of the egg.

The term “transgene” refers to heterologous nucleotide sequence inserted into an avian genome in accordance with the invention. “Transgene” can specifically refer to an exogenous coding sequence, an exogenous coding sequence linked to an exogenous promoter or other regulatory sequence, all nucleotide sequence between two retoroviral LTRs and/or retroviral LTRs and nucleotide sequence between the LTRs wherein the LTRs are of a retrovirus used to introduce the transgene.

The term “optimized” is used in the context of “optimized coding sequence”, wherein the most frequently used codons for each particular amino acid found in the egg white proteins ovalbumin, lysozyme, ovomucoid, and ovotransferrin are used in the design of the optimized human interferon-α 2b (IFN-α 2b) polynucleotide sequence that is inserted into vectors of the present invention. More specifically, the DNA sequence for optimized human IFN-α 2b is based on the hen oviduct optimized codon usage and is created using the BACKTRANSLATE program of the Wisconsin Package, Version 9.1 (Genetics Computer Group Inc., Madison, Wis.) with a codon usage table compiled from the chicken (Gallus gallus) ovalbumin, lysozyme, ovomucoid, and ovotransferrin proteins. For example, the percent usage for the four codons of the amino acid alanine in the four egg white proteins is 34% for GCU, 31% for GCC, 26% for GCA, and 8% for GCG. Therefore, GCU is used as the codon for the majority of alanines in an optimized coding sequence. The vectors containing the gene for the optimized human protein are used to produce transgenic avians that express transgenic poultry derived protein in their tissues and eggs.

As used herein, the term “subject” encompasses mammals and non-mammals. Examples of mammals include, but are not limited to, humans, chimpanzees, apes monkeys, cattle, horses, sheep, goats, swine; rabbits, dogs, cats, rats, mice, guinea pigs, and the like.

As used herein, the term “therapeutically effective amount” refers to any amount of a compound which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The term “treat,” “treating” or “treatment” refers to methods of alleviating, abating or ameliorating a disease or condition symptoms, preventing additional symptoms, ameliorating or preventing the underlying causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

### LAL Compositions

The invention is generally drawn to compositions comprising enzymes useful for therapy, for example, in the treatment of lysosomal storage diseases. In one aspect, the invention is drawn to lysosomal storage disease enzymes such as LAL with a glycosylation pattern that renders the molecule amenable for internalization by certain cell types. Also included in the invention are recombinant human proteins including LAL in isolated or purified form. The isolation of the lysosomal storage disease enzymes (such as LAL) can be accomplished by methodologies readily apparent to a practitioner skilled in the art of protein purification.

In one embodiment, the invention is directed to lysosomal storage disease enzymes including, but not limited to LAL, having an N-linked glycosylation pattern described herein.

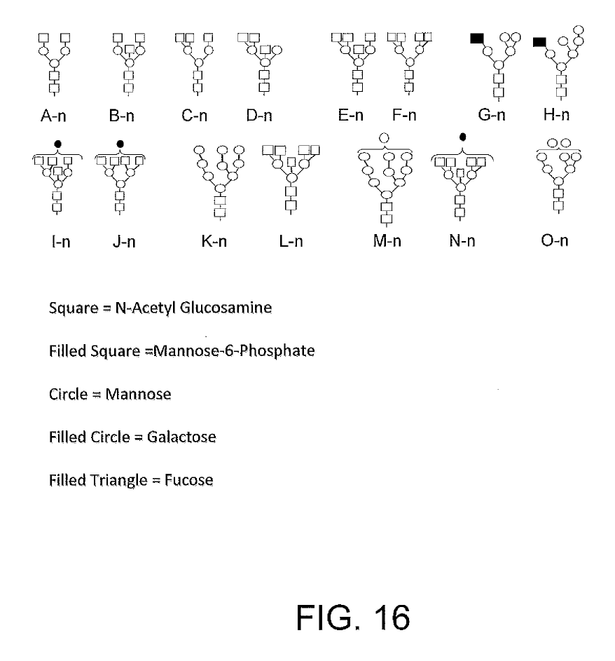

In one aspect, the compositions disclosed herein comprise human LAL wherein a substantial percentage of the human LAL contain a mannose-6-phosphate glycan moiety, which can serve as a ligand for internalization by the mannose-6-phosphate receptor on the surface of cells found, for example, on hepatocytes. In one embodiment, 30% or more, for example, at least 30%, at least 40%, at least 50%, at least 60%, at least 70%, at least 80%, at least 90%, at least 95%, at least 97%, or at least 99%, of the LAL contained in the composition contains at least one mannose-6-phosphate moiety. The mannose-6-phosphate moiety can be found, for example, on an N-glycan structure located at one or more residues selected from the group consisting of Asn¹⁵, Asn⁵¹, Asn⁸⁰, Asn¹⁴⁰, Asn²⁵² and Asn³⁰⁰ of SEQ ID NO:2. Glycan structures containing mannose-6-phosphate moieties include, for example, G-n and H-n shown in FIG. 16.

The recombinant human LAL according to the present invention contains multiple N-linked carbohydrate chains (e.g., about 5 or 6 carbohydrate chains). N-linked glycosylation structures at each of the five or six sites can be selected from one of A-n, B-n, C-n, D-n, E-n, F-n, G-n, H-n, I-n, J-n, K-n, L-n, M-n, N-n and O-n as shown in FIG. 16

Also described herein are a mixture of LAL molecules (e.g., more than one LAL molecule can be present in a mixture such as the LAL molecules set forth in SEQ ID NOs: 2, 3, 4 and 19) wherein some or all of the LAL molecules have one or more glycosylation structures selected from Structure A-n, Structure B-n, Structure C-n, Structure D-n, Structure E-n, Structure F-n, Structure G-n, Structure H-n, Structure I-n, Structure J-n, Structure K-n, Structure L-n, Structure M-n, Structure N-n and Structure O-n (FIG. 16). In one embodiment, the mixture of lysosomal acid lipase molecules is purified or isolated, for example, isolated from an egg or purified or isolated from egg white produced in a transgenic avian.

The invention also includes an individual LAL molecule comprising a Structure A-n. The invention also includes an individual LAL molecule comprising a Structure B-n. The invention also includes an individual LAL molecule comprising a Structure C-n. The invention also includes an individual LAL molecule comprising a Structure D-n. The invention also includes an individual LAL molecule comprising a Structure E-n. The invention also includes an individual LAL molecule comprising a Structure F-n. The invention also includes an individual LAL molecule comprising a Structure G-n. The invention also includes an individual LAL molecule comprising a Structure H-n. The invention also includes an individual LAL molecule comprising a Structure I-n. The invention also includes an individual LAL molecule comprising a Structure J-n. The invention also includes an individual LAL molecule comprising a Structure K-n. The invention also includes an individual LAL molecule comprising a Structure L-n. The invention also includes an individual lysosomal acid lipase molecule comprising a Structure M-n. The invention also includes an individual LAL molecule comprising a Structure N-n. The invention also includes an individual LAL molecule comprising a Structure O-n.

N-linked oligosaccharides attached to human LAL according to the present invention have a paucity of terminal sialic acid and galactose residues. That is, only minor amounts of the N-linked oligosaccharide structures are terminally sialylated and few galactose residues are present as well. Further, terminal N-Acetyl Glucosamine (GlcNAc) is present extensively on the N-linked oligosaccharide structures of the LAL described herein. As such, LAL produced in accordance with the invention can be targeted to cells such as monocyte macrophages and Kupffer cells.

One aspect of the invention provides compositions of LAL having essentially no sialic acid. In another aspect, the compositions disclosed herein comprise recombinant human LAL wherein a substantial percentage of the human LAL does not contain a sialic acid moiety in any of its N-glycan structures, which can interfere with internalization of the enzyme into cells. In one embodiment, 15% or less, for example, 10% or less, 5% or less, 2% or less, 1% or less, or essentially none, of the LAL contained in the composition contains a sialic acid moiety in any of its N-glycan structures.

In another embodiment, about 95% or more of the N-linked oligosaccharides present on the individual LAL molecule of the invention do not contain sialic acid. In another embodiment, about 90% or more of the N-linked oligosaccharides present on the individual LAL molecule of the invention do not contain sialic acid. In another embodiment, about 80% or more of the N-linked oligosaccharides present on the individual LAL molecule of the invention do not contain sialic acid. In another embodiment, more than about 70% or more of the N-linked oligosaccharides present on the individual LAL molecule of the invention do not contain sialic acid.

In still another embodiment, essentially none of the N-linked oligosaccharides structure types present on the LAL molecules of the invention contain sialic acid. In another embodiment, about 90% or more of the N-linked oligosaccharides structure types found to be associated with the LAL molecules of the invention do not contain sialic acid. For example, if there are 20 oligosaccharide structure types, then 18 or more of the structure types do not contain sialic acid. In another embodiment, about 80% or more of the N-linked oligosaccharides structure types found to be associated with the LAL molecules of the invention do not contain sialic acid. In another embodiment, about 70% or more of the N-linked oligosaccharides structure types found to be associated with the LAL molecules of the invention do not contain sialic acid. In another embodiment, about 60% or more of the N-linked oligosaccharides structure types found to be associated with the LAL molecules of the invention do not contain sialic acid. In another embodiment, about 50% or more of the N-linked oligosaccharides structure types found to be associated with the LAL molecules of the invention do not contain sialic acid.

According to one aspect of the invention, LAL as described herein contain high levels of terminal N-Acetyl Glucosamine. In one aspect, about 95% or more of the N-linked oligosaccharides present on the individual LAL molecule of the invention contain a terminal N-Acetyl Glucosamine. In another embodiment, about 90% or more of the N-linked oligosaccharides present on the individual LAL molecule of the invention contain a terminal N-Acetyl Glucosamine. In another embodiment, about 80% or more of the N-linked oligosaccharides present on the individual LAL molecule of the invention contain a terminal N-Acetyl Glucosamine. In another embodiment, about 70% or more of the N-linked oligosaccharides present on the individual LAL molecule of the invention contain a terminal N-Acetyl Glucosamine. In another embodiment, about 60% or more of the N-linked oligosaccharides present on the individual LAL molecule of the invention contain a terminal N-Acetyl Glucosamine. In another embodiment, about 50% or more of the N-linked oligosaccharides present on the individual LAL molecule of the invention contain a terminal N-Acetyl Glucosamine.

In one embodiment, all of the N-linked oligosaccharides structure types present on the LAL molecules of the invention contain a terminal N-Acetyl Glucosamine. In another embodiment, about 90% or more of the N-linked oligosaccharides structure types present on the LAL molecules of the invention contain a terminal N-Acetyl Glucosamine. For example, if there are 20 oligosaccharide structure types, then 18 or more of the structure types do not contain a terminal N-Acetyl Glucosamine. In another embodiment, about 80% or more of the N-linked oligosaccharides structure types present on the LAL molecules of the invention contain a terminal N-Acetyl Glucosamine. In another embodiment, about 70% or more of the N-linked oligosaccharides structure types present on the LAL molecules of the invention contain a terminal N-Acetyl Glucosamine. In another embodiment, about 60% or more of the N-linked oligosaccharides structure types present on the LAL molecules of the invention contain a terminal N-Acetyl Glucosamine. In another embodiment, about 50% or more of the N-linked oligosaccharides structure types present on the LAL molecules of the invention contain a terminal N-Acetyl Glucosamine.

In another aspect of the invention, the compositions disclosed herein comprise human LAL wherein a substantial percentage of the human LAL does not contain a fucose moiety in any of its N-glycan structure. In one embodiment, 50% or less, for example, 50% or less, 40% or less, 30% or less, 20% or less, 10% or less, 5% or less, 2% or less, 1% or less, or essentially none, of the LAL contained in the composition contains a fucose moiety in any of its N-glycan structure.

In one embodiment, fucose is essentially not present on the N-linked oligosaccharide structures of the LAL produced in accordance of the invention. In another embodiment, about 95% or more of the N-linked oligosaccharides present on the individual LAL molecule of the invention do not contain fucose. In another embodiment, about 90% or more of the N-linked oligosaccharides present on the individual LAL molecule of the invention do not contain fucose. In another embodiment, about 85% or more of the N-linked oligosaccharides present on the individual LAL molecule of the invention do not contain fucose. In another embodiment, about 80% or more of the N-linked oligosaccharides present on the individual LAL molecule of the invention do not contain fucose. In another embodiment, about 70% or more of the N-linked oligosaccharides present on the individual LAL molecule of the invention do not contain fucose. In another embodiment, about 60% or more of the N-linked oligosaccharides present on the individual LAL molecule of the invention do not contain fucose. In another embodiment, about 50% or more of the N-linked oligosaccharides present on the LAL of the invention do not contain fucose.

In one embodiment, essentially none of the N-linked oligosaccharides structure types present on the LAL molecules of the invention contain fucose. In another embodiment, about 95% or more of the N-linked oligosaccharides structure types present on the LAL molecules of the invention do not contain fucose. For example, if there are 20 oligosaccharide structure types, then 19 or more of the structure types do not contain fucose. In another embodiment, about 90% or more of the N-linked oligosaccharides structure types present on the LAL molecules of the invention do not contain fucose. In another embodiment, about 85% or more of the N-linked oligosaccharides structure types present on the LAL molecules of the invention do not contain fucose. In another embodiment, about 80% or more of the N-linked oligosaccharides structure types present on the LAL molecules of the invention do not contain fucose. In another embodiment, about 70% or more of the N-linked oligosaccharides structure types present on the LAL molecules of the invention do not contain fucose.

As discussed above, certain monosaccharides are abundantly present in LAL molecules produced in accordance with the present invention. The total monosaccharide species analyzed includes fucose, N-acetyl galactosamine, N-acetyl glucosamine, galactose, glucose, mannose, mannose-6-phosphate, N-acetyl neuraminic acid and N-glycolyl neuraminic acid. Fucose can be present between about 0% and about 1% of the total monosaccharide composition. N-acetyl galactosamine can be present between about 0% and about 1% of the total monosaccharide composition. N-acetyl glucosamine can be present between about 35% and about 50% of the total monosaccharide composition. Galactose can be present between about 1-10% of the total monosaccharide composition. Glucose is present at 0% of the total monosaccharide composition. Mannose is present between about 32% and about 50% of the total monosaccharide composition. Mannose-6-phosphate is present between about 1% and about 11% of the total monosaccharide composition.

In one embodiment, LAL produced in accordance with the present invention do not contain any xylose. In addition, because there is essentially no N-acetylgalactosamine (GalNac) in LAL produced in accordance with the invention, one aspect of the invention includes a composition of LAL having no O-linked glycosylation.

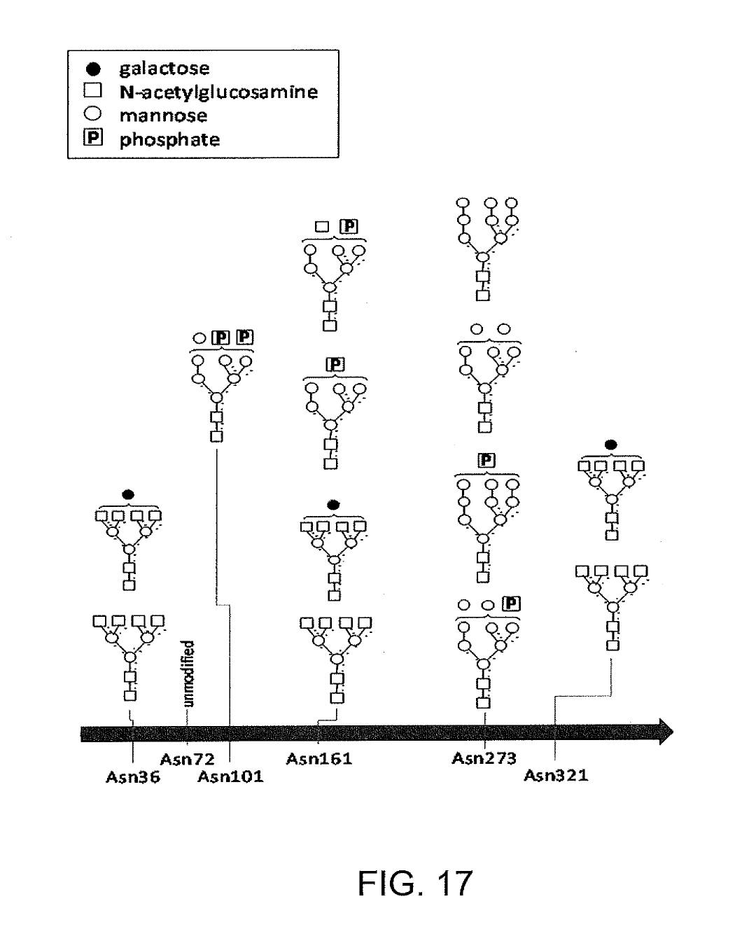

LAL has 6 potential sites in its amino acid sequence for N-linked glycosylation, for example, Asn³⁶, Asn⁷², Asn¹⁰¹, Asn¹⁶¹, Asn²⁷³, and Asn³²¹ as in SEQ ID NO:1. Five of these, Asn³⁶, Asn¹⁰¹, Asn¹⁶¹, Asn²⁷³ and Asn³²¹ are glycosylated while Asn⁷² can be unglycosylated or substantially unglycosylated (substantially unglycosylated means in a mixture of LAL molecules, fewer Asn⁷² are glycosylated than any of Asn³⁶, Asn¹⁰¹, Asn¹⁶¹, Asn²⁷³ and Asn³²¹) (see FIG. 17). Accordingly, one aspect of the invention is a composition of LAL which is unglycosylated and/or substantially unglycosylated at Asn⁷². LAL having a glycosylated Asn⁷² is within the scope of the invention. The positions of Asn described herein are based on the LAL amino acid sequence set forth in SEQ ID NO:1. It will be apparent to those skilled in the art that the numbering of Asn (i.e., the position of asparagine) can vary depending on individual LAL molecule and be readily determined in other LAL molecules such as those whose amino acid sequences are set forth in SEQ ID NOs:2, 3, 4 and 19.

The LAL molecules produced in accordance with the present invention contain N-glycan structures comprising a mixture of bi-, tri- and tetraantennary structures with N-acetylglucosamine, mannose and mannose-6-phosphate (M6P) as the major sugars (FIGS. 16 and 17). According to one aspect of the invention, M6P-modified N-glycans reside at least at Asn¹⁰¹, Asn¹⁶¹ and Asn²⁷³. Thus, one embodiment of the present invention includes a composition of LAL having M6P-modified N-glycans residing at any one of Asn¹⁰¹, Asn¹⁶¹ or Asn²⁷³. In yet another embodiment, the present invention includes a composition of LAL having M6P-modified N-glycans residing at Asn²⁷³. In another embodiment, the present invention includes a composition of LAL having monophosphorylated N-glycans (M6P) residing at any one of Asn¹⁰¹, Asn¹⁶¹ or Asn²⁷³. In yet another embodiment, the present invention includes a composition of LAL having monophosphorylated N-glycans residing at Asn¹⁶¹ and Asn²⁷³. In yet another embodiment, the present invention includes a composition of LAL having monophosphorylated N-glycans residing at Asn¹⁰¹ and Asn²⁷³. In one specific embodiment, a LAL produced in accordance with the present invention can contain bisphosphorylated mannose (bis-M6P) at Asn¹⁰¹.

The LAL molecules produced in accordance with the present invention contain reduced levels of galactose (e.g., “Gal”). One aspect of the present invention includes a composition of LAL having terminal galactose at any one of Asn³⁶, Asn¹⁶¹ or Asn³²¹. In yet another embodiment, the present invention includes a composition of LAL having terminal galactose at Asn³⁶ and Asn¹⁶¹. In yet another embodiment, the present invention includes a composition of LAL having terminal galactose at Asn¹⁶¹ and Asn³²¹. In yet another embodiment, the present invention includes a composition of LAL having terminal galactose at Asn³⁶ and Asn³²¹. In yet another embodiment, the present invention includes a composition of LAL having terminal galactose at Asn³⁶, Asn¹⁶¹ and Asn³²¹. In yet another embodiment, the present invention includes a composition of LAL having no terminal galactose.

Various types of N-glycans were found in LAL at different N-linked glycosylation sites. The N-glycan structures include a mixture of bi-, tri- and tetraantennary structures with N-acetylglucosamine, mannose and mannose-6-phosphate (M6P) as the major sugars. Specifically, in one embodiment of the present invention, LAL contains an N-glycan structure selected from GlcNAc4Man3GlcNAc2 or Gal1GlcNAc4Man3GlcNAc2 at the first N-linked glycosylation site (e.g., Asn³⁶ as in SEQ ID NO:1). In another embodiment, LAL contains no glycosylation or is substantially unglycosylated at the second N-linked glycosylation site (e.g., Asn⁷² as in SEQ ID NO:1). In yet another embodiment, LAL contains Phos2Man7GlcNAc2 at its third N-linked glycosylation site (e.g., Asn¹⁰¹ as in SEQ ID NO:1). In yet another embodiment, LAL contains a N-glycan structure selected from Phos1Man6GlcNAc2, GlcNAc1Phos1Man6GlcNAc2, Man3GlcNAc2, GlcNAc2Man3GlcNAc2, GlcNAc3Man3GlcNAc2, GlcNAc4Man3GlcNAc2, or Gal1GlcNAc4Man3GlcNAc2 at its fourth N-linked glycosylation site (e.g., Asn¹⁶¹ as in SEQ ID NO:1). In yet another embodiment, LAL contains a N-glycan structure selected from Man7GlcNAc2, Man8GlcNAc2, Man9GlcNAc2, Phos1Man8GlcNAc2, or Phos1Man9GlcNAc2 at its fifth N-linked glycosylation site (e.g., Asn²⁷³ as in SEQ ID NO:1). In yet another embodiment, LAL contains a N-glycan structure selected from GlcNAc2Man3GlcNAc2, GlcNAc3Man3GlcNAc2, GlcNAc4Man3GlcNAc2, Gal1GlcNAc4Man3GlcNAc2, GlcNAc5Man3GlcNAc2, Gal1GlcNAc5Man3GlcNAc2, GlcNAc6Man3GlcNAc2, or Gal1GlcNAc6Man3GlcNAc2 at its sixth N-linked glycosylation site (e.g., Asn³²¹ as in SEQ ID NO:1).

According to certain aspects of the invention, compositions of LAL include LAL glycosylated at Asn³⁶, Asn⁷², Asn¹⁰¹, Asn¹⁶¹, Asn²⁷³ and Asn³²¹ of SEQ ID NO:1 (or corresponding Asparagine residues within SEQ ID NOs: 2, 3, 4, and 19) with one N-glycan at the designated Asn position as shown below:

a) at Asn³⁶, GlcNAc4Man3GlcNAc2, or
Gal1GlcNAc4Man3GlcNAc2;

b) at Asn⁷², no glycosylation;

c) at Asn¹⁰¹, Phos2Man7GlcNAc2;

d) at Asn¹⁶¹, Phos1Man6GlcNAc2,

GlcNAc1Phos1Man6GlcNAc2;
Man3 GlcNAc2;
GlcNAc2Man3GlcNAc2;
GlcNAc3Man3 GlcNAc2;
GlcNAc4Man3GlcNAc2, or
Gal1GlcNAc4Man3 GlcNAc2;

e) at Asn²⁷³, Man7GlcNAc2,
Man8GlcNAc2,
Man9GlcNAc2,
Phos1 Man8GlcNAc2, or
Phos1 Man9 GlcNAc2; and

f) at Asn³²¹, GlcNAc2Man3GlcNAc2,
GlcNAc3Man3GlcNAc2,
GlcNAc4Man3GlcNAc2,
Gal1GlcNAc4Man3GlcNAc2,
GlcNAc5Man3GlcNAc2,
Gal1GlcNAc5Man3GlcNAc2,
GlcNAc6Man3GlcNAc2, or
Gal1GlcNAc6Man3GlcNAc2,
   where Man=mannose,

GlcNAc=N-Acetyl Glucosamine,

Phos=phosphate, and

Gal=galactose.

In one embodiment, Gal1GlcNAc4Man3GlcNAc2 can be found as a glycan component at any one of Asn³⁶, Asn¹⁶¹ or Asn³²¹ in LAL produced in accordance with the invention. In one specific embodiment, Gal1GlcNAc4Man3GlcNAc2 can be found as a glycan component of Asn³⁶, Asn¹⁶¹ and Asn³²¹.

In the LAL of the present invention, Asn¹⁰¹ and Asn²⁷³ display the high-mannose-type having about 6 to about 10 mannose molecules (MAN6-MAN10 as described herein) as a major component. Accordingly, one aspect of the present invention includes a composition of LAL having a high mannose structure at Asn¹⁰¹ or Asn²⁷³. In another embodiment, a composition of LAL of the invention can comprise a N-glycan structure having at least 6 mannose at Asn¹⁰¹ or Asn²⁷³. In another embodiment, a composition of LAL contains a N-glycan having 7, 8 or 9 mannose at Asn¹⁰¹ or Asn²⁷³. In yet another embodiment, the present invention includes a composition of LAL having 7, 8 or 9 mannose at Asn¹⁰¹ and Asn²⁷³. In yet another embodiment, the present invention includes a composition of LAL having 7, 8 or 9 mannose at Asn¹⁰¹ and/or Asn²⁷³ and at least one of the mannose is phosphorylated.

It is to be understood that the glycosylation sites and the numbers associated with Asn described above is based on the amino acid sequence of LAL set forth in SEQ ID NO:1 and that the glycosylation profiles described above in context of SEQ ID NO:1 also apply to LAL molecules set forth in SEQ ID NOs: 2, 3, 4 and 19 though the numbering of corresponding Asn may vary depending on LAL molecule. For example, Asn³⁶ in SEQ ID NO:1 corresponds to Asn¹⁵ in SEQ ID NO:2, Asn¹³ in SEQ ID NO:3, Asn¹⁰ in SEQ ID NO:4 and Asn⁹ in SEQ ID NO:19. Asn⁷² in SEQ ID NO:1 corresponds to Asn⁵¹ in SEQ ID NO:2, Asn⁴⁹ in SEQ ID NO:3, Asn⁴⁶ in SEQ ID NO:4 and Asn⁴⁵ in SEQ ID NO:19. Asn¹⁰¹ in SEQ ID NO:1 corresponds to Asn⁸⁰ in SEQ ID NO:2, Asn⁷⁸ in SEQ ID NO:3, Asn⁷⁵ in SEQ ID NO:4 and Asn⁷⁴ in SEQ ID NO:19. Asn¹⁶¹ in SEQ ID NO:1 corresponds to Asn¹⁴⁰ in SEQ ID NO:2, Asn¹³⁸ in SEQ ID NO:3, Asn¹³⁵ in SEQ ID NO:4 and Asn¹³⁴ in SEQ ID NO:19. Asn²⁷³ of SEQ ID NO:1 corresponds to Asn²⁵² in SEQ ID NO:2, Asn²⁵⁰ in SEQ ID NO:3, Asn²⁴⁷ in SEQ ID NO:4 and Asn²⁴⁶ in SEQ ID NO:19. Asn³²¹ of SEQ ID NO:1 corresponds to Asn³⁰⁰ in SEQ ID NO:2, Asn²⁹⁸ in SEQ ID NO:3, Asn²⁹⁵ in SEQ ID NO:4 and Asn²⁹⁴ in SEQ ID NO:19.

For example, in one embodiment, the LAL is N-linked glycosylated at least at one position selected from the group consisting of Asn¹⁵, Asn⁵¹, Asn⁸⁰, Asn¹⁴⁰, Asn²⁵² and Asn³⁰⁰ of SEQ ID NO:2. In another embodiment, the LAL is N-linked glycosylated at Asn¹⁵, Asn⁸⁰, Asn¹⁴⁰, Asn²⁵² and Asn³⁰⁰ of SEQ ID NO:2. In yet another embodiment, N-glycan structures of LAL of SEQ ID NO:2 have no xylose while less than 15%, 10%, 5%, or 1% of N-glycan structures contain sialic acid; less than 50%, 40%, 30%, 20%, 10%, 5% or 1% of N-glycan structures contain fucose; and at least 30%, 50%, 60%, 70%, 80%, 90% and 95% of N-glycan structures contain phosphorylated mannose (M6P). S

In one embodiment, the LAL is N-linked glycosylated at least at one position selected from the group consisting of Asn¹³, Asn⁴⁹, Asn⁷⁸, Asn¹³⁸, Asn²⁵⁰ and Asn²⁹⁸ of SEQ ID NO:3. In another embodiment, the LAL is N-linked glycosylated at Asn¹³, Asn⁷⁸, Asn¹³⁸, Asn²⁵⁰ and Asn²⁹⁸ of SEQ ID NO:3. In yet another embodiment, N-glycan structures of LAL of SEQ ID NO:3 have no xylose while less than 15%, 10%, 5%, or 1% of N-glycan structures contain sialic acid; less than 50%, 40%, 30%, 20%, 10%, 5% or 1% of N-glycan structures contain fucose; and at least 30%, 50%, 60%, 70%, 80%, 90% and 95% of N-glycan structures contain phosphorylated mannose (M6P).

In one embodiment, the LAL is N-linked glycosylated at least at one position selected from the group consisting of Asn¹⁰, Asn⁴⁶, Asn⁷⁵, Asn¹³⁵, Asn²⁴⁷ and Asn²⁹⁵ of SEQ ID NO:4. In another embodiment, the LAL is N-linked glycosylated at Asn¹⁰, Asn⁷⁵, Asn¹³⁵, Asn²⁴⁷ and Asn²⁹⁵ of SEQ ID NO:4. In yet another embodiment, N-glycan structures of LAL of SEQ ID NO:4 have no xylose while less than 15%, 10%, 5%, or 1% of N-glycan structures contain sialic acid; less than 50%, 40%, 30%, 20%, 10%, 5% or 1% of N-glycan structures contain fucose; and at least 30%, 50%, 60%, 70%, 80%, 90% and 95% of N-glycan structures contain phosphorylated mannose (M6P).

In one embodiment, the LAL is N-linked glycosylated at least at one position selected from the group consisting of Asn⁹, Asn⁴⁵, Asn⁷⁴, Asn¹³⁴, Asn²⁴⁶ and Asn²⁹⁴ of SEQ ID NO:19. In another embodiment, the LAL is N-linked glycosylated at Asn⁹, Asn⁷⁴, Asn¹³⁴, Asn²⁴⁶ and Asn²⁹⁴ of SEQ ID NO:19. In yet another embodiment, N-glycan structures of LAL of SEQ ID NO:4 have no xylose while less than 15%, 10%, 5%, or 1% of N-glycan structures contain sialic acid; less than 50%, 40%, 30%, 20%, 10%, 5% or 1% of N-glycan structures contain fucose; and at least 30%, 50%, 60%, 70%, 80%, 90% and 95% of N-glycan structures contain phosphorylated mannose (M6P).

The composition according to the present invention can be produced a number of ways, including by use of transgenic avians, transgenic fish, transgenic mammals, for example, transgenic goats or in transgenic plants, such as tobacco and duck weed (Lemna minor) and certain types of cell culture.

The present invention also contemplates compositions comprising PEGylated LAL. LAL enzyme as described herein can be PEGylated as disclosed, for example, in U.S. Patent publication No. 20070092486, published Apr. 26, 2007, the disclosure of which is incorporated it its entirety herein by reference.

In one embodiment, the derived glycosylation pattern is obtained through expression specialized expression systems, for example, from avian oviduct cells, for example, tubular gland cells. For example, glycosylation patterns disclosed herein have been demonstrated to be present on lysosomal storage disease enzymes produced in oviduct cells of an avian such as a chicken in accordance with the present invention.

Proteins produced in accordance with the invention can be purified from egg white by any useful procedure such as those apparent to a practitioner of ordinary skill in the art of protein purification. For example, the human LAL (hLAL) produced in transgenic avians in accordance with the invention can be purified from egg white by methods apparent to practitioners of ordinary skill in the art of protein purification. An example of a purification protocol for LAL present in egg white is described in the Examples.

The invention includes the eggs and egg white and the avians (e.g., chicken turkey and quail) that lay the eggs and produce the egg white containing lysosomal acid lipase molecules of the invention comprising one or more of the glycosylation structures disclosed herein.

### Expression of LAL in Avians

Disclosed herein are vectors and methods for the stable introduction of exogenous nucleic acid sequences into the genome of avians to express desired proteins such as those which benefit (e.g., attain an increased efficacy) from the addition of mannose-6-phosphate such as lysosomal enzymes including, without limitation, lysosomal acid lipase (LAL) and other proteins such as those specifically disclosed herein. In particular, transgenic avians are produced which express exogenous sequences in their oviducts and which deposit exogenous proteins, such as pharmaceutical proteins, into their eggs. Avian eggs that contain such exogenous proteins are also described herein. Also disclosed herein are novel forms of LAL which are efficiently expressed in the oviduct of transgenic avians and deposited into avian eggs.

One aspect of the invention relates to compositions containing LAL, i.e., LAL molecules produced in accordance with the invention. In a particularly useful embodiment, the LAL is purified or isolated. For example, the LAL has been removed from the contents of a hard shell egg laid by a transgenic avian. In one particularly useful embodiment, the LAL is human LAL. In one embodiment, the LAL of the invention has a glycosylation pattern resulting from the LAL being produced in an oviduct cell of an avian. For example, the compositions can contain a mixture of LAL molecules produced in avians, for example, chickens, in accordance with the invention and isolated from egg white. In one useful embodiment, the LAL containing compositions are pharmaceutical formulations.

In one aspect, the invention is drawn to compositions containing isolated LAL molecules, for example, human LAL molecules, wherein the LAL is produced in an avian which contains a transgene encoding the LAL. In one embodiment, the LAL is produced in an oviduct cell (e.g., a tubular gland cell) of a transgenic avian (e.g., transgenic chicken) and the LAL is isolated from egg white of the transgenic avian. In one embodiment, the LAL is glycosylated in the oviduct cell (e.g., tubular gland cell) of the bird, for example, a chicken.

In another aspect, methods for producing exogenous proteins such as lysosomal storage disease enzymes, for example, LAL, in specific tissues of avians, are provided. Such exogenous proteins may be expressed in the oviduct, blood and/or other cells and tissues of the avian. In one embodiment, transgenes are introduced into embryonic blastodermal cells, for example, near stage X, to produce a transgenic avian, such that the protein of interest is expressed in the tubular gland cells of the magnum of the oviduct, secreted into the lumen, and deposited into the egg white of a hard shell egg. A transgenic avian so produced can carry the transgene in its germ line providing transmission of the exogenous transgene to the avian's offspring stably in a Mendelian fashion.

The present invention encompasses methods of producing exogenous protein such as LAL in an avian oviduct. The methods may include a first step of providing a, vector that contains a coding sequence and a promoter operably linked to the coding sequence, so that the promoter can effect expression of the nucleic acid in the avian oviduct. Transgenic cells and/or tissues can be produced, wherein the vector is introduced into avian embryonic blastodermal cells, either freshly isolated, in culture, or in an embryo, so that the vector sequence is inserted into the avian genome. A mature transgenic avian which expresses the exogenous protein such as LAL in its oviduct can be derived from the transgenic cells and/or tissue.

In one aspect of the invention, production of a transgenic avian is accomplished by transduction of embryonic blastodermal cells with replication-defective or replication-competent retroviral particles carrying the transgene between the 5′ and 3′ LTRs of the retroviral rector. For instance, an avian leukosis virus (ALV) retroviral vector or a murine leukemia virus (MLV) retroviral vector may be used which comprises a modified pNLB plasmid containing an exogenous gene that is inserted downstream of a segment of a promoter region. An RNA copy of the modified retroviral vector, packaged into viral particles, can be used to infect embryonic blastoderms which develop into transgenic avians.

Another aspect of the invention provides a vector which includes a coding sequence and a promoter in operational and positional relationship such that the coding sequence is expressed in an avian oviduct. Such vectors include, but are not limited to, an avian leukosis virus (ALV) retroviral vector, a murine leukemia virus (MLV) retroviral vector, and a lentivirus vector. In addition, the vector may be a nucleic acid sequence which includes an LTR of an avian leukosis virus (ALV) retroviral vector, a murine leukemia virus (MLV) retroviral vector, or a lentivirus vector. The promoter is sufficient for effecting expression of the coding sequence in the avian oviduct. The coding sequence codes for an exogenous protein which is deposited into the egg white of a hard shell egg. As such, the coding sequence codes for exogenous proteins such as transgenic poultry derived proteins such as transgenic poultry derived lysosomal acid lipase (TPD LAL).

In one embodiment, vectors used in the methods of the invention contain a promoter which is particularly suited for expression of exogenous proteins in avians and their eggs. As such, expression of the exogenous coding sequence may occur in the oviduct and blood of the transgenic avian and in the egg white of its avian egg. The promoters include, but are not limited to, a cytomegalovirus (CMV) promoter, a rous-sarcoma virus (RSV) promoter, a β-actin promoter (e.g., a chicken β-actin promoter), a murine leukemia virus (MLV) promoter, a mouse mammary tumor virus (MMTV) promoter, an ovalbumin promoter, a lysozyme promoter, a conalbumin promoter, an ovomucoid promoter, an ovomucin promoter, and an ovotransferrin promoter. Optionally, the promoter may be a segment of at least one promoter region, such as a segment of the ovalbumin, lysozyme, conalbumin, ovomucoid, ovomucin, and ovotransferrin promoter region. In one embodiment, the promoter is a combination or a fusion of one or more promoters or a fusion of a portion of one or more promoters such as ovalbumin, lysozyme, conalbumin, ovomucoid, ovomucin, and ovotransferrin promoters.

In one embodiment, the vector includes a signal peptide coding sequence which is operably linked to the coding sequence, so that upon translation in a cell, the signal peptide directs secretion of the exogenous protein expressed by the vector, such as human LAL, into the egg white of a hard shell egg.

One aspect of the invention provides for coding sequences for exogenous proteins produced as disclosed herein wherein the coding sequence is codon optimized for expression in an avian, for example, in a chicken. Codon optimization may be determined from the codon usage of at least one, and preferably more than one, protein expressed in an avian cell (e.g., a chicken cell). For example, the codon usage may be determined from the nucleic acid sequences encoding the proteins ovalbumin, lysozyme, ovomucin and ovotransferrin of chicken. For example, the DNA coding sequence for the exogenous protein may be codon optimized using the BACKTRANSLATE® program of the Wisconsin Package, version 9.1 (Genetics Computer Group, Inc., Madison, Wis.) with a codon usage table compiled from the chicken (Gallus gallus) ovalbumin, lysozyme, ovomucoid, and ovotransferrin proteins.

One important aspect of the present invention relates to avian hard shell eggs (e.g., chicken hard shell eggs) which contain an exogenous peptide or protein including, but not limited to, a human LAL. The exogenous peptide or protein such as human LAL may be encoded by a transgene of a transgenic avian. Often, the exogenous peptide or protein (e.g., LAL) is glycosylated. The protein may be present in any useful amount. In one embodiment, the protein is present in an amount in a range of between about 0.01 μg per hard-shell egg and about 1 gram per hard-shell egg. In another embodiment, the protein is present in an amount in a range of between about 1 μg per hard-shell egg and about 1 gram per hard-shell egg. For example, the protein may be present in an amount in a range of between about 10 μg per hard-shell egg and about 1 gram per hard-shell egg (e.g., a range of between about 10 μg per hard-shell egg and about 400 milligrams per hard-shell egg).

In one embodiment, the exogenous protein of the invention is present in the egg white of the egg. In one embodiment, the protein is present in an amount in a range of between about 1 ng per milliliter of egg white and about 0.2 gram per milliliter of egg white. For example, the protein may be present in an amount in a range of between about 0.1 μg per milliliter of egg white and about 0.2 grain per milliliter of egg white (e.g., the protein may be present in an amount in a range of between about 1 μg per milliliter of egg white and about 100 milligrams per milliliter of egg white. In one embodiment, the protein is present in an amount in a range of between about 1 μg per milliliter of egg white and about 50 milligrams per milliliter of egg white. For example, the protein may be present in an amount in a range of about 1 μg per milliliter of egg white and about 10 milligrams per milliliter of egg white (e.g., the protein may be present in an amount in a range of between about 1 μg per milliliter of egg white and about 1 milligrams per milliliter of egg white). In one embodiment, the protein is present in an amount of more than 0.1 μg per milliliter of egg white. In one embodiment, the protein is present in an amount of more than 0.5 μg per milliliter of egg white. In one embodiment, the protein is present in an amount of more than 1 μg per milliliter of egg white. In one embodiment, the protein is present in an amount of more than 1.5 μg per milliliter of egg white.

The avians of the invention which produce exogenous proteins disclosed herein (e.g., LAL) which are developed from the blastodermal cells into which the vector has been introduced are the G0 generation and can be referred to as “founders.” Founder birds are typically chimeric for each inserted transgene. That is, only some of the cells of the G0 transgenic bird contain the transgene(s). The G0 generation typically is also hemizygous for the transgene(s). The G0 generation may be bred to non-transgenic animals to give rise to G1 transgenic offspring which are also hemizygous for the transgene and contain the transgene(s) in essentially all of the bird's cells. The G1 hemizygous offspring may be bred to non-transgenic animals giving rise to G2 hemizygous offspring or may be bred together to give rise to G2 offspring homozygous for the transgene. Substantially all of the cells of birds which are positive for the transgene that are derived from G1 offspring contain the transgene(s). In one embodiment, hemizygotic G2 offspring from the same line can be bred to produce G3 offspring homozygous for the transgene. In one embodiment, hemizygous G0 or G1 animals, for example, are bred together to give rise to homozygous G1 offspring containing two copies of the transgene(s) in each cell of the animal. These are merely examples of certain useful breeding methods and the present invention contemplates the employment of any useful breeding method such as those known to individuals of ordinary skill in the art.

In one embodiment, the invention provides for the LAL to be isolated. That is, the LAL contained in the composition may be an isolated LAL. For example, the LAL may be isolated from egg white. The isolated LAL may be LAL molecules having a variety of glycosylation structures among the LAL molecules.

By the methods of the present invention, transgenes can be introduced into avian embryonic blastodermal cells to produce a transgenic chicken, transgenic turkey, transgenic quail and other avian species, that carry the transgene in the genetic material of its germ-line tissue in order to produce proteins of the invention. The blastodermal cells are typically stage cells, or the equivalent thereof, and in one embodiment are near stage X.

Some vectors useful in carrying out the methods of the present invention are described herein. In one embodiment, the coding sequence and the promoter of the vector are both positioned between 5′ and 3′ LTRs before introduction into blastodermal cells. In one embodiment, the vector is retroviral and the coding sequence and the promoter are both positioned between the 5′ and 3′ LTRs of the retroviral vector. In one useful embodiment, the LTRs or retroviral vector is derived from the avian leukosis virus (ALV), murine leukemia virus (MLV), or lentivirus.

In one embodiment, vectors are used for transfecting blastodermal cells and generating stable integration into the avian genome contain a coding sequence and a promoter in operational and positional relationship to express the coding sequence in the tubular gland cell of the magnum of the avian oviduct, wherein the exogenous protein such as an lysosomal enzyme (e.g., LAL) is deposited in the egg white of a hard shell egg.

The promoter may optionally be a segment of the ovalbumin promoter region which is sufficiently large to direct expression of the coding sequence in the tubular gland cells. Truncating the ovalbumin promoter and/or condensing the critical regulatory elements of the ovalbumin promoter so that it retains sequences required for expression in the tubular gland cells of the magnum of the oviduct, while being small enough that it can be readily incorporated into vectors is included within the scope of the invention. In one embodiment, a segment of the ovalbumin promoter region may be used. This segment comprises the 5′-flanking region of the ovalbumin gene.

The promoter may also be a promoter that is largely, but not entirely, specific to the magnum, such as the lysozyme promoter. The promoter may also be a mouse mammary tumor virus (MMTV) promoter. Alternatively, the promoter may be a constitutive promoter (e.g., a cytomegalovirus (CMV) promoter, a rous-sarcoma virus (RSV) promoter, a murine leukemia virus (MLV) promoter, etc.). In one embodiment, the promoter is a cytomegalovirus (CMV) promoter, a MDOT promoter, a rous-sarcoma virus (RSV) promoter, a murine leukemia virus (MLV) promoter, a mouse mammary tumor virus (MMTV) promoter, an ovalbumin promoter, a lysozyme promoter, a conalbumin promoter, an ovomucoid promoter, an ovomucin promoter and/or an ovotransferrin promoter. Optionally, the promoter may be at least one segment of a promoter region, such as a segment of the ovalbumin, lysozyme, conalbumin, ovomucoid, ovomucin, and ovotransferrin promoter region.

In one method of transfecting blastodermal cells, a packaged retroviral-based vector is used to deliver the vector into embryonic blastodermal cells so that the vector is integrated into the avian genome.

Useful retrovirus for randomly introducing a transgene into the avian genome is the replication-deficient avian leucosis virus (ALV), the replication-deficient murine leukemia virus (MLV), or the lentivirus. In one embodiment, a pNLB vector is modified by inserting a region of the ovalbumin promoter and one or more exogenous genes between the 5′ and 3′ long terminal repeats (LTRs) of the retrovirus genome. The invention contemplates that any coding sequence placed downstream of a promoter that is active in tubular gland cells can be expressed in the tubular gland cells. For example, the ovalbumin promoter can be expressed in the tubular gland cells of the oviduct magnum because the ovalbumin promoter drives the expression of the ovalbumin protein and is active in the oviduct tubular gland cells.

Any of the vectors described herein can also optionally include a coding sequence encoding a signal peptide that directs secretion of the protein expressed by the vector's coding sequence from the tubular gland cells of the oviduct. This aspect effectively broadens the spectrum of exogenous proteins that may be deposited in avian eggs using the methods described herein. Where an exogenous protein would not otherwise be secreted, the vector containing the coding sequence is modified to comprise a DNA sequence comprising about 60 bp encoding a signal peptide from the lysozyme gene. The DNA sequence encoding the signal peptide is inserted in the vector such that it is located at the N-terminus of the protein encoded by the DNA.

Another aspect of the invention involves the use of internal ribosome entry site (IRES) elements in any of the vectors of the present invention to allow the translation of two or more proteins from a dicistronic or polycistronic mRNA. The IRES units are fused to 5′ ends of one or more additional coding sequences which are then inserted into the vectors at the end of the original coding sequence, so that the coding sequences are separated from one another by an IRES.

In one embodiment when using an IRES, post-translational modification of the product is facilitated because one coding sequence can encode an enzyme capable of modifying the other coding sequence product. For example, the first coding sequence may encode collagen which would be hydroxylated and made active by the enzyme encoded by the second coding sequence wherein an IRES is employed as is understood in the art.

In another aspect, the coding sequences of vectors used in any of the methods of the present invention are provided with a 3′ untranslated region (3′ UTR) to confer stability to the RNA produced. When a 3′ UTR is added to a retroviral vector, the orientation of the promoter, gene X and the 3′ UTR must be reversed in the construct, so that the addition of the 3′ UTR does not interfere with transcription of the full-length genomic RNA. In one embodiment, the 3′ UTR may be that of the ovalbumin or lysozyme genes, or any 3′ UTR that is functional in a magnum cell, i.e., the SV40 late region.

In one embodiment, a constitutive promoter is used to express the coding sequence of a transgene in the avian. In this case, expression is not limited to the magnum; expression also occurs in other tissues within the avian (e.g., blood). The use of such a transgene, which includes a constitutive promoter and a coding sequence, is particularly suitable for effecting or driving the expression of a protein in the oviduct and the subsequent secretion of the protein into the egg.

Transducing particles (i.e., transduction particles) are produced for the vector and titered to determine the appropriate concentration that can be used to inject embryos. Avian eggs are windowed according to the Speksnijder procedure (U.S. Pat. No. 5,897,998, the disclosure of which is incorporated in its entirety herein by reference), and eggs are injected with transducing particles. Eggs hatch about 21 days after injection and male birds are selected for breeding. In order to screen for G0 roosters which contain the transgene in their sperm, DNA is extracted from rooster sperm samples. The G0 roosters with the highest levels of the transgene in their sperm samples are bred to nontransgenic hens by artificial insemination. Blood DNA samples are screened for the presence of the transgene. The serum of transgenic roosters is tested for the presence of exogenous protein. If the exogenous protein is confirmed, the sperm of the transgenic roosters is used for artificial insemination of nontransgenic hens. A certain percent of the offspring then contains the transgene (e.g., more than 50%). When exogenous protein is present in eggs produced in accordance with the present invention the protein may be isolated. The protein may also be tested for biological activity.

The methods of the invention which provide for the production of exogenous protein in the avian oviduct and the production of eggs which contain exogenous protein involve an additional step subsequent to providing a suitable vector and introducing the vector into embryonic blastodermal cells so that the vector is integrated into the avian genome. The subsequent step involves deriving a mature transgenic avian from the transgenic blastodermal cells produced in the previous steps. Mature transgenic avians can be obtained from the cells of a blastodermal embryo which has been transfected or transduced with the vector directly within the embryo. The resulting embryo is allowed to develop and the chick allowed to mature.

The transgenic avian produced from blastodermal cells is known as a founder. Some founders will carry the transgene in tubular gland cells in the magnum of their oviducts. These avians will express the exogenous protein encoded by the transgene in their oviducts. The exogenous protein may also be expressed in other tissues (e.g., blood) in addition to the oviduct. If the exogenous protein contains the appropriate signal sequence(s), it will be secreted into the lumen of the oviduct and into the egg white of the egg.

Some founders are germ-line founders. A germ-line founder is a founder that carries the transgene in genetic material of its germ-line tissue, and may also carry the transgene in oviduct magnum tubular gland cells that express the exogenous protein. Therefore, in accordance with the invention, the transgenic avian may have tubular gland cells expressing the exogenous protein, and the offspring of the transgenic avian may also have oviduct magnum tubular gland cells that express the exogenous protein. Alternatively, the offspring express a phenotype determined by expression of the exogenous gene in specific tissue(s) of the avian. In one embodiment, the transgenic avian is a chicken or a turkey.

### Pharmaceutical Compositions & Therapeutic Methods

While it is possible that, for use in therapy, therapeutic proteins produced as described herein may be administered in raw form, it is preferable to administer the therapeutic proteins as part of a pharmaceutical formulation. Therefore, further provided are pharmaceutical formulations comprising poultry derived glycosylated therapeutic proteins such as LAL or a pharmaceutically acceptable derivative thereof together with one or more pharmaceutically acceptable carriers thereof and, optionally, other therapeutic and/or prophylactic ingredients and methods of administering such pharmaceutical formulations. The carrier(s) must be “acceptable” in the sense of being compatible with the other ingredients of the formulation and not deleterious to the recipient thereof. Methods of treating a patient (e.g., quantity of pharmaceutical protein administered, frequency of administration and duration of treatment period) using pharmaceutical compositions of the invention can be determined using standard methodologies known to physicians of skill in the art.

Compositions comprising carriers, including composite molecules, are formulated by well-known conventional methods (see, for example, Remington's Pharmaceutical Sciences, 14th Ed., Mack Publishing Co., Easton, Pa.), the entire teachings of which are incorporated herein by reference. The carrier may comprise a diluent. In one embodiment, the pharmaceutical carrier can be a liquid and the recombinant human LAL can be in the form of a solution. The pharmaceutical carrier can be wax, fat, or alcohol. In one embodiment, the wax- or fat-based carrier does not contain ester. In another embodiment, the pharmaceutically acceptable carrier may be a solid in the form of a powder, a lyophilized powder, or a tablet. In one embodiment, the carrier may comprise a liposome or a microcapsule.

The pharmaceutical formulations include those suitable for administration by injection including intramuscular, sub-cutaneous and intravenous administration. Pharmaceutical formulations include those suitable for oral, rectal, nasal, topical (including buccal and sub-lingual), vaginal or parenteral. The pharmaceutical formulations also include those for administration by inhalation or insufflation. The formulations may, where appropriate, be conveniently presented in discrete dosage units and may be prepared by any of the methods well known in the art of pharmacy. The methods of producing the pharmaceutical formulations typically include the step of bringing the therapeutic protein into association with liquid carriers or finely divided solid carriers or both and then, if necessary, shaping the product into the desired formulation.

Pharmaceutical formulations suitable for oral administration may conveniently be presented as discrete units such as capsules, cachets or tablets each containing a predetermined amount of the active ingredient; as a powder or granules; as a solution; as a suspension; or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils) or preservatives.

LAL may also be formulated for parenteral administration (e.g., by injection, for example, bolus injection or continuous infusion) and may be presented in unit dose form in ampoules, pre-filled syringes, small volume infusion or in multi-dose containers with an added preservative. The therapeutic proteins can be injected by, for example, subcutaneous injections, intramuscular injections, and intravenous infusions or injections.

The LAL may take such forms as suspensions, solutions, or emulsions in oily or aqueous vehicles, and may contain formulatory agents such as suspending, stabilizing and/or dispersing agents. It is also contemplated that the therapeutic protein may be in powder form, obtained by aseptic isolation of sterile solid or by lyophilization from solution, for constitution with a suitable vehicle, e.g., sterile, pyrogen-free water, before use.

For intravenous infusions or injection, the LAL produced in accordance of the invention can be formulated as an aqueous suspension or solution. Excipients suitable for the formulation for intravenous infusion or injection can include one of the following: trisodium citrate dehydrate, citric acid and human serum albumin. The pharmaceutical formulation can also include other suitable excipients well known in the art used for other products for lysosomal storage disorders. The pH of LAL produced in accordance with the invention is maintained between about 5.6 and about 6.2. Preferably, the pH of the LAL formulation is maintained at 5.9±0.2.

For topical administration to the epidermis, the therapeutic proteins of the invention produced according to the invention may be formulated as ointments, creams or lotions, or as a transdermal patch. Ointments and creams may, for example, be formulated with an aqueous or oily base with the addition of suitable thickening and/or gelling agents. Lotions can be formulated with an aqueous or oily base and can also contain one or more emulsifying agents, stabilizing agents, dispersing agents, suspending agents, thickening agents or coloring agents.

Formulations suitable for topical administration in the mouth include lozenges comprising active ingredient in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutical formulations suitable for rectal administration wherein the carrier is a solid are most preferably represented as unit dose suppositories. Suitable carriers include cocoa butter and other materials commonly used in the art, and the suppositories may be conveniently formed by a mixture of the active compound with the softened or melted carrier(s) followed by chilling and shaping in molds.

Formulations suitable for vaginal administration may be presented as pessaries, tampons, creams, gels, pastes, foams or sprays containing in addition to the active ingredient, such carriers as are known in the art to be appropriate.

For intra-nasal administration the therapeutic proteins of the invention may be used as a liquid spray or dispersible powder or in the form of drops. Drops may be formulated with an aqueous or non-aqueous base also comprising one or more dispersing agents, solubilizing agents or suspending agents. Liquid sprays are conveniently delivered from pressurized packs.

For administration by inhalation, therapeutic proteins according to the invention may be conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

For administration by inhalation or insufflation, the therapeutic proteins according to the invention may take the form of a dry powder composition, for example, a powder mix of the compound and a suitable powder base such as lactose or starch. The powder composition may be presented in unit dosage form in, for example, capsules or cartridges or, e.g., gelatin or blister packs from which the powder may be administered with the aid of an inhalator or insufflator.

When desired, the above described formulations adapted to give sustained release of the active ingredient, may be employed.

The pharmaceutical compositions described herein may also contain other active ingredients such as antimicrobial agents, or preservatives.

In addition, it is contemplated that the therapeutic proteins disclosed herein may be used in combination with other therapeutic agents. For example, the invention provides methods for pretreatment with a pharmaceutically effective dose of an antihistamine to minimize or prevent potential infusion-related anaphylactic reactions. For example, the antihistamine may be any pharmaceutically acceptable antihistamine (e.g. diphenhydramine) as disclosed herein and as known in the art. In one embodiment, the antihistamine is administered in a dose between about 1 mg and about 10 mg per kilogram of body weight. For example, the antihistamine may be administered in a dose of about 5 mg per kilogram. In one embodiment, the antihistamine is administered between about 10 minutes and about 90 minutes, for example, about 30 minutes to about 60 minutes, prior to administration of lysosomal acid lipase using an ambulatory system connected to the vascular access port. In one embodiment, the dose of diphenhydramine effectively counteracts potential anaphylactic infusion reactions.

Immunosuppresants such as antihistamines, corticosteroids, sirolimus, voclosporin, ciclosporin, methotrexate, IL-2 receptor directed antibodies, T-cell receptor directed antibodies, TNF-α directed antibodies or fusion proteins (infliximab, etanercept or adalimumab), CTLA4-Ig (e.g., abatacept), anti-OX-40 antibodies can also be administered before, during or after LAL administration if an anaphylactic reaction or adverse immune response is experienced by the patient.

The invention also contemplates therapy involving administration of LAL-containing compositions in combination with one or more cholesterol lowering agents (e.g., HMG-CoA reductase inhibitors). Non-limiting examples of such agents include: atorvastatin (Lipitor® and Torvast®), fluvastatin (Lescol®), lovastatin (Mevacor®, Altocor®, Altoprev®), pitavastatin (Livalo®, Pitava®), pravastatin (Pravachol®, Selektine®, Lipostat®), rosuvastatin (Crestor®) and simvastatin (Zocor®, Lipex®).

Compositions or proteins described herein can be used to treat a variety of conditions. For example, there are conditions for which treatment therapies are known to practitioners of skill in the art. The present invention contemplates that the therapeutic proteins (e.g., LAL) produced in an avian system containing a poultry derived glycosylation pattern can be employed to treat such conditions. That is, the treatment of conditions known to be treatable by conventionally produced therapeutic proteins by using therapeutic proteins produced as described herein is also contemplated. For example, LAL produced as described herein can be used to treat conditions resulting from or associated with LAL deficiency or insufficiency (collectively, “LAL deficiency”), such as Wolman disease and cholesteryl ester storage disease (CESD). As described herein, LAL deficiency also contemplates conditions in which expression of LAL is reduced due to a condition (e.g., a genetic mutation), physiological or environmental factors which leads to a reduction or deficiency of LAL produced in the body. LAL produced as described herein can also be used to treat other conditions such as atherosclerosis, fatty liver disease, non-alcoholic fatty liver disease, nonalcoholic steatohepatitis (NASH) and cirrhosis. LAL produced as described herein can also be used to treat other conditions such as those disclosed in U.S. Pat. No. 6,849,257, issued Feb. 1, 2005, the disclosure of which is incorporated in its entirety herein by reference, U.S. publication No. 2009/0297496, published Dec. 3, 2009; US publication No. 2004/0223960, published Nov. 11, 2004; US publication No. 2007/0264249, published Nov. 15, 2009, the disclosures of which (i.e., the disclosures of each of these four patent publications) are incorporated in their entirety herein by reference.

It is also contemplated that LAL produced as disclosed herein can be used to treat certain specific conditions including pancreatitis, for example, chronic pancreatitis and/or acute pancreatitis as well as alcohol induced pancreatic injury such as alcohol induced pancreatitis.

LAL produced by any useful method, such as the ones disclosed herein, is contemplated for use to treat diseases due to alcohol induced cell injury including, but not limited to, those alcohol induced cell injuries that result in accumulation of lipid esters in body tissue such as, but not limited to, liver, spleen, gut and cardiovascular tissue. The invention also contemplates the treating of malabsorption by administering LAL.

One aspect of the invention is drawn to methods of treating a patient comprising administering to a patient a therapeutically effective amount of a composition comprising recombinant human LAL as described herein. The patient can be suffering or diagnosed with any number of conditions, including those associated with LAL deficiency. In one embodiment, the therapeutically effective amount is an amount that increases the red blood cell count in a patient by a desired amount. It is contemplated that LAL produced in accordance with the invention can be used to treat chronic kidney disease, for example, where tissues fail to sustain production of lysosomal acid lipase.

It is also contemplated that LAL produced by any useful method may be useful for the treatment of patients with Tangier disease and familial hypoalphalipoproteinemia. Tangier disease/familial hypoalphalipoproteinemia is associated with the accumulation of cholesterol esters in macrophages accompanied by hepatosplenomegaly and/or lymphadenopathy along with low levels of high-density lipoproteins (HDL) which can be treated by the administration of LAL. For example, without wishing to limit the invention to any particular theory or mechanism of operation, it is believed that impaired LAL activity decreases ABCA1 expression and conversely an increased LAL activity obtained by the administration of LAL to a patient with Tangier disease/familial hypoalphalipoproteinemia will increase ABCA1 expression to overcome the effects of an ABCA1 gene with a reduced functional activity as a result of polymorphism.

For the treatment of a condition, generally, the dosage administered can vary depending upon known factors such as age, health and weight of the recipient, type of concurrent treatment, frequency of treatment, and the like. Usually, a dosage of active ingredient can be between about 0.0001 and about 10 milligrams per kilogram of body weight. Precise dosage, frequency of administration and time span of treatment can be determined by a physician skilled in the art of administration of the respective therapeutic protein.

In addition, it has been discovered that dosages of 1 mg/kg and less can be effective in treating LAL deficiencies. The present invention provides methods of treating conditions comprising administering to a mammal (e.g. a patient, preferably a human patient) a therapeutically effective dose of lysosomal acid lipase between one time every 5 days and one time every 25 days, for example, between one time every 7 days and one time every 14 days. In one embodiment, the dose of lysosomal acid lipase administered is between about 0.1 mg and about 50 mg per kilogram of body weight, for example, the dose may be between about 1 mg and 5 mg per kilogram.

In one particularly useful embodiment, the invention provides methods of treating a condition by administering a dose of lysosomal acid lipase of between about 0.1 mg and 1.0 mg per kilogram of body weight in accordance with any therapeutically effective dosage regime such as those described herein.

The invention provides methods for treating any complication of LAL deficiency which may benefit from administering a therapeutically effective dose of LAL. In one embodiment, malabsorption and growth failure may be treated in accordance with the methods described herein. In another embodiment, complications seen in LAL deficiency patients including but not restricted to hepatomegaly and liver dysfunction may be treated using the methods provided herein.

The invention provides for treatment with recombinant LAL (e.g. recombinant human LAL) that can be produced by any useful protein expression system, for example, transgenic mammals and avians as is understood in the art. Other protein expression systems may include, but are not limited to, cell culture, bacteria, and plant systems.

The invention encompasses the administration of recombinant LAL as a part of a pharmaceutically acceptable composition by any route which may achieve the intended therapeutic effect, as determined by a physician skilled in the art. In one embodiment, the LAL may be administered by intravenous infusion over a period of about five hours. For example, the infusion may be facilitated by an ambulatory infusion pump connected to a vascular access port (e.g. a Port-a-Cath).

The invention also includes monitoring clinical and pathological presentation of the conditions, for example, Wolman Disease and CESD, in the mammal (e.g. the human patient). In one embodiment, the assessments consist of but are not limited to: lipid analysis, chest x-ray, liver function tests, stool chart, plasma mevalonic acid, immunogenicity, plasma lysosomal acid lipase, chitotriosidase, PARC, portal hypertension, anthropometry, volume and characterization of the liver, spleen, and gastrointestinal tract using, for example, imaging technology. For example, the aforementioned imaging technology may consist of ultrasound, magnetic resonance imaging, and nuclear magnetic resonance spectroscopy.

### EXAMPLES

The present invention is further exemplified by the following examples. The examples are for illustrative purpose only and are not intended, nor should they be construed as limiting the invention in any manner.

### Example 1

### Construction of Vector (pALVIN-OVR1-I-hLAL-dSA) Carrying Recombinant Human Lysosomal Acid Lipase (rhLAL) Coding Sequence

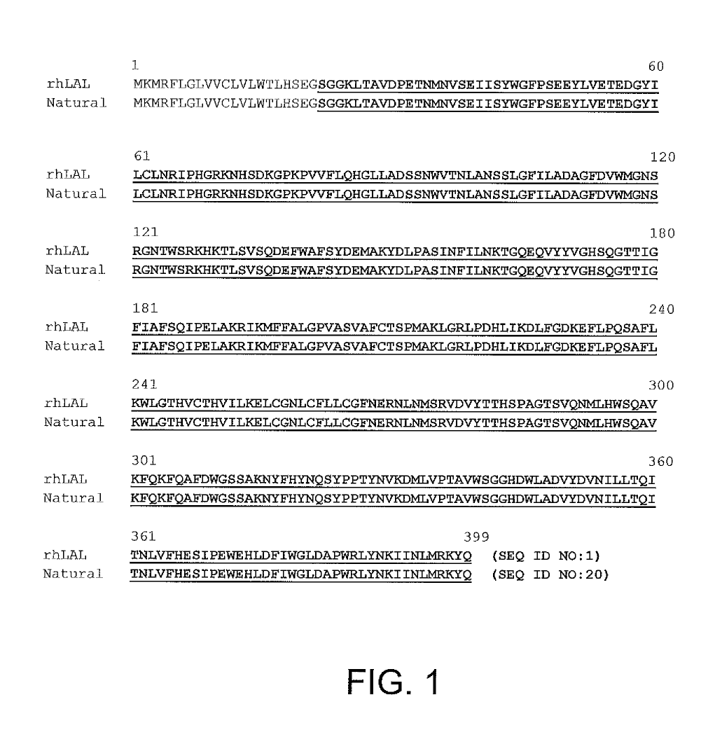

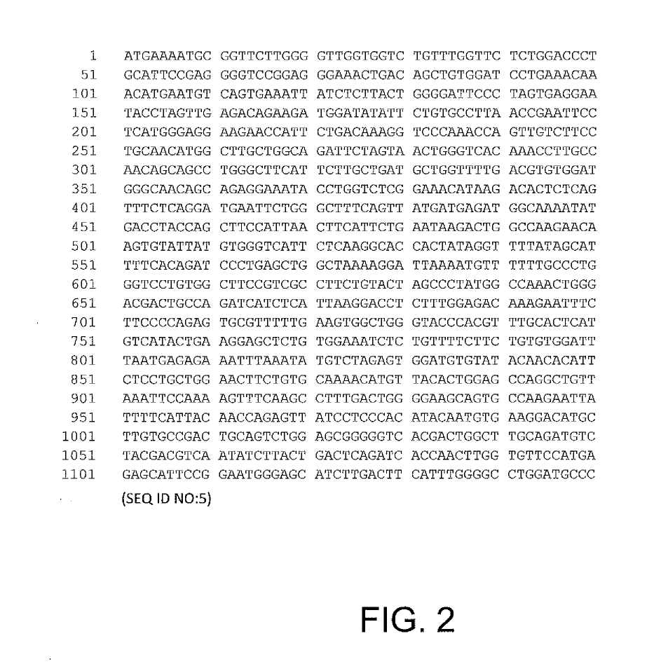

The nucleotide sequence of the hLAL gene in the pALVIN-OVR1-I-hLAL-dSA vector encodes a protein that is identical to the amino acid sequence of the protein produced by the human lysosomal acid lipase gene (GenBank Accession, NP_{—}000226) (FIG. 1). Transcription of this sequence and subsequent translation of the resultant mRNA produces a 399 amino acid precursor protein, which is processed to a mature 378 amino acid protein identical to human LAL (GenBank Accession, NP_{—}000226) (FIG. 1) as set forth in SEQ ID NO:1. Expression of the hLAL gene (see FIG. 2 for the cDNA sequence) in this Example is controlled by non-coding elements derived from the ovalbumin gene including enhancer, promoter, intronic, and 5′ and 3′ untranslated sequences. The ovalbumin gene produces ovalbumin, the major protein constituent of egg white. Activity of the chicken ovalbumin promoter is very specific to the cells within the chicken oviduct that produce egg white; expression in other tissues is minimal.

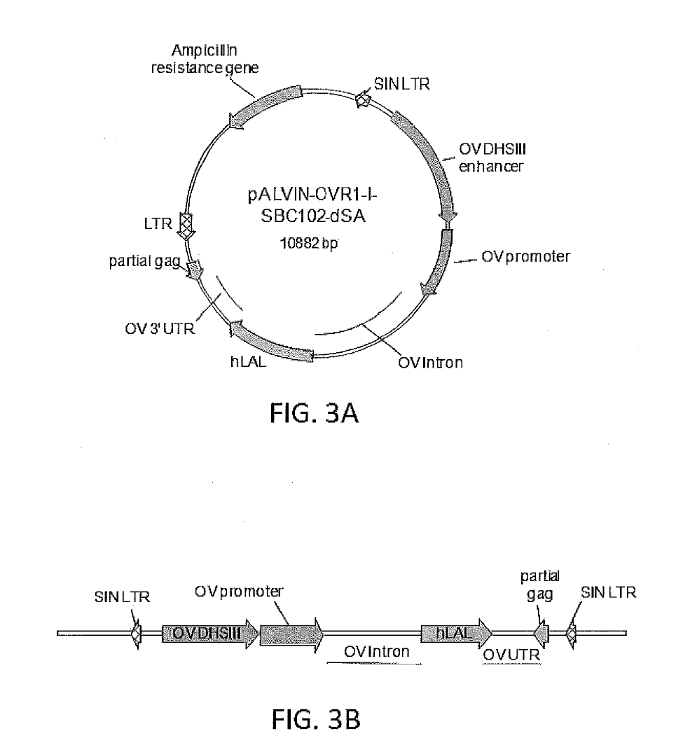

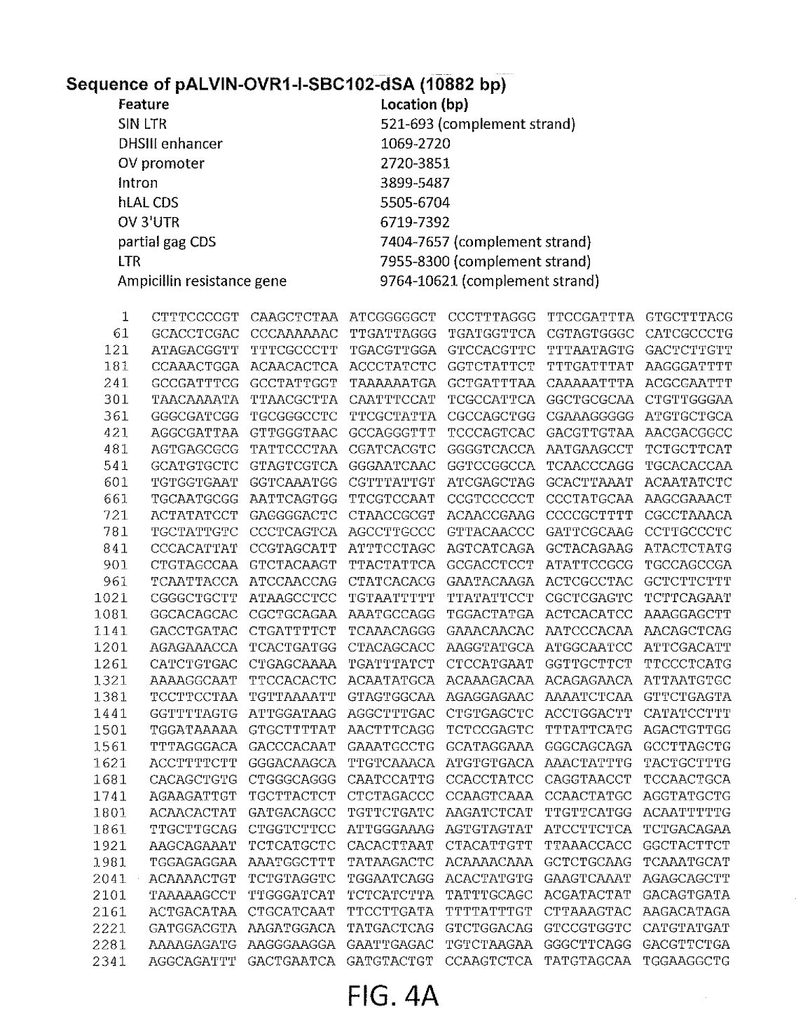

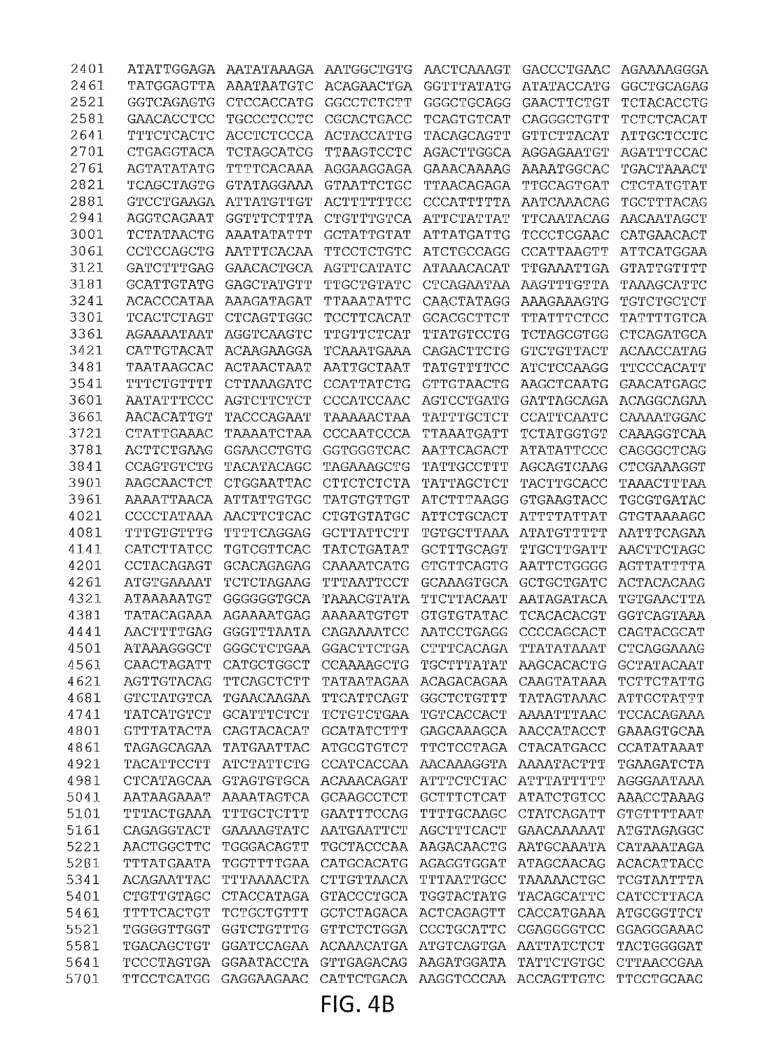

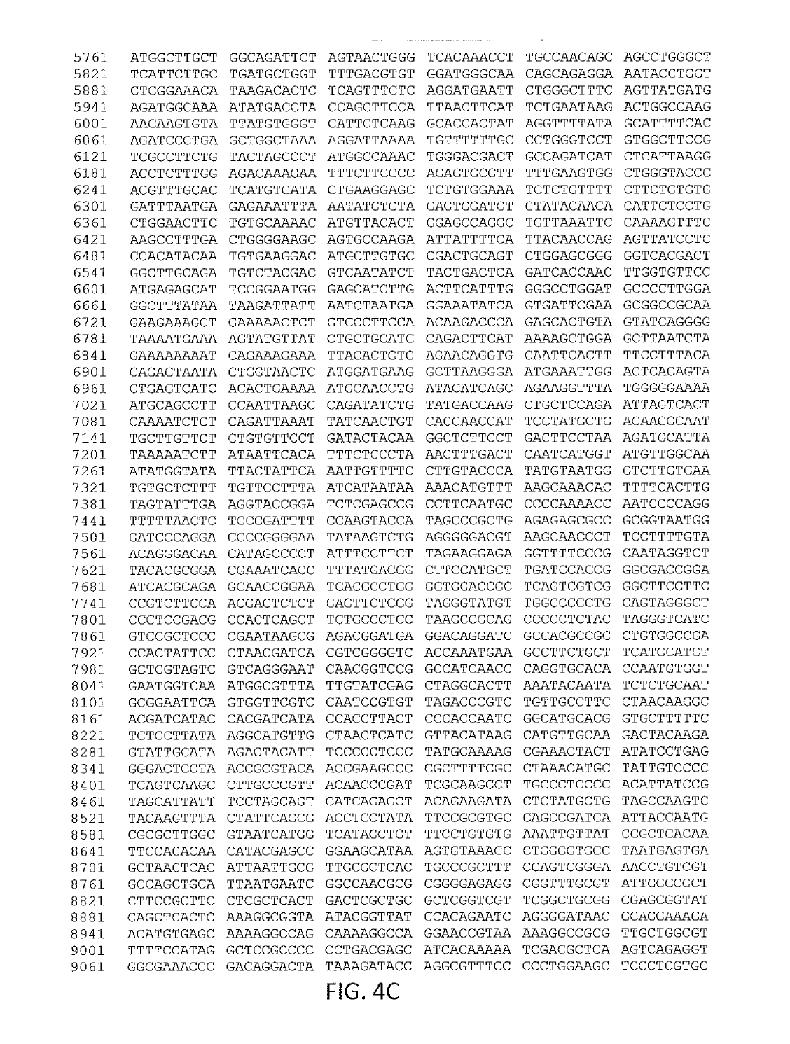

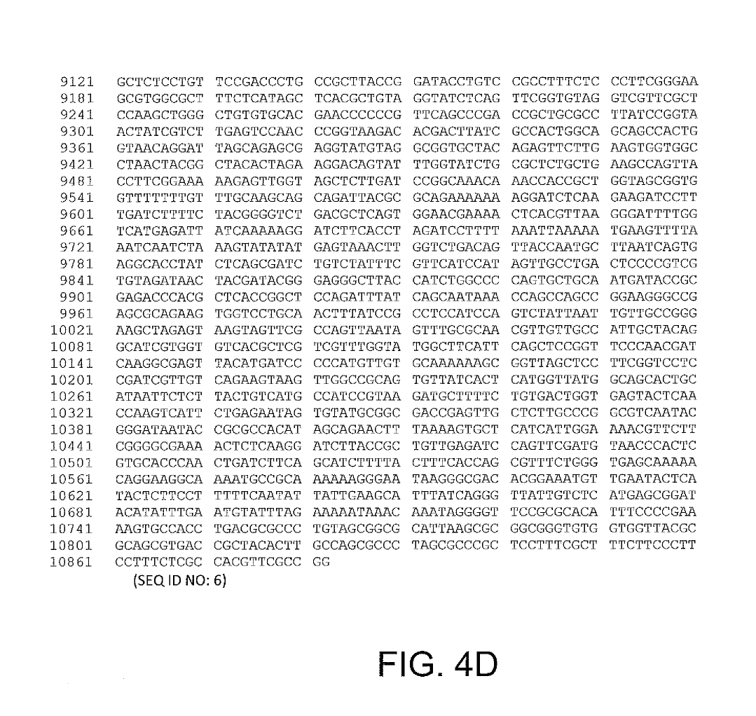

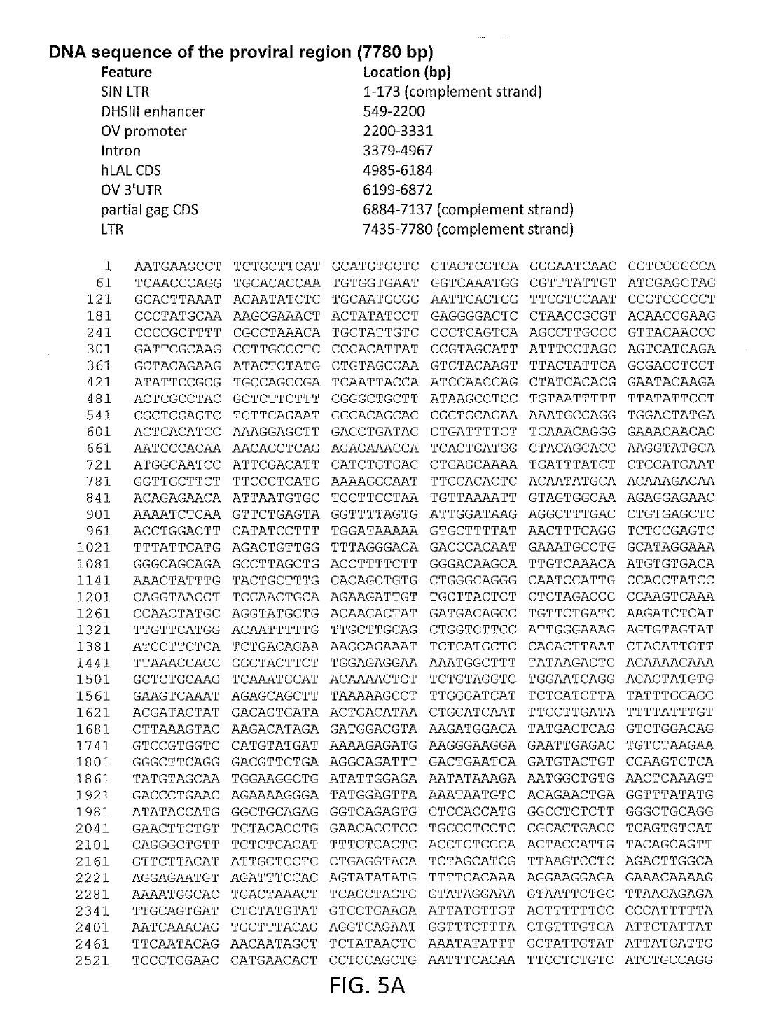

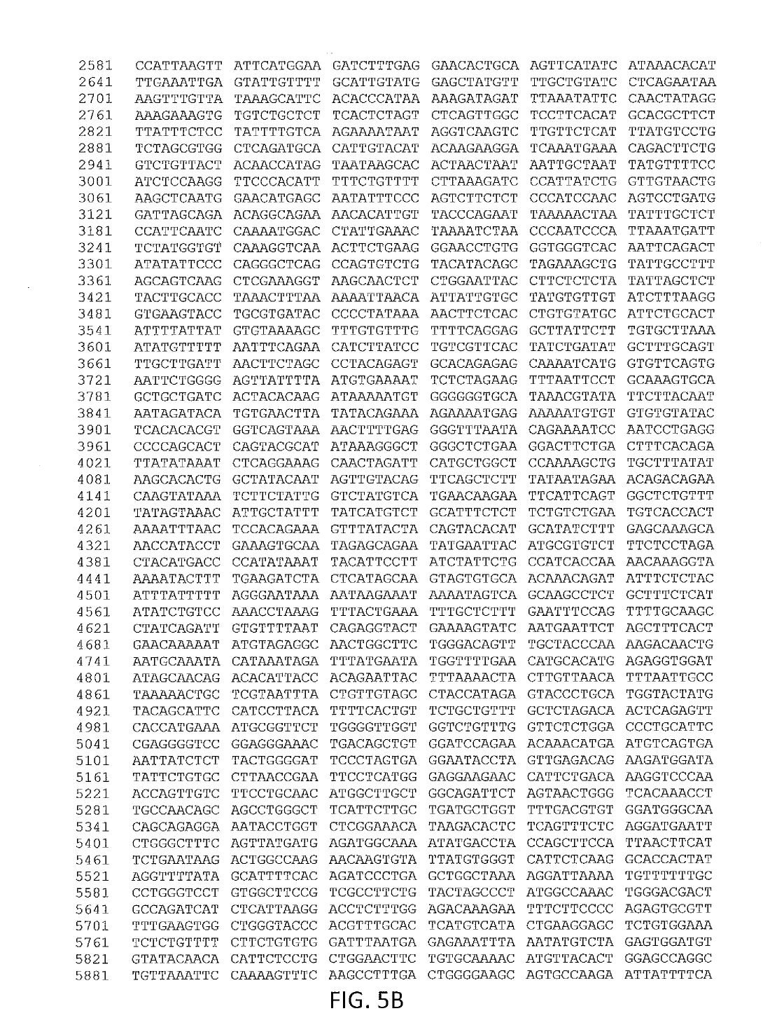

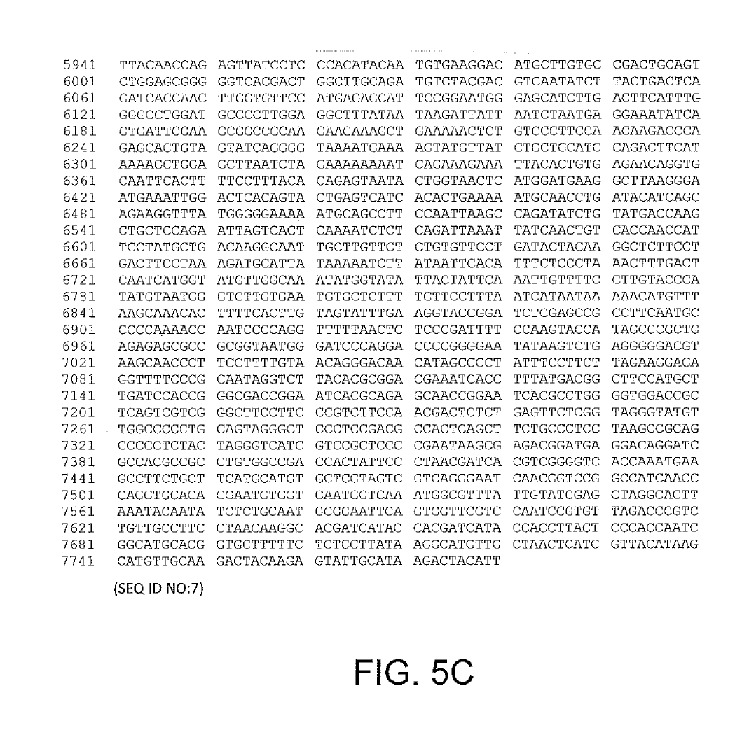

The plasmid vector pALVIN-OVR1-I-hLAL-dSA (FIG. 3A; the nucleotide sequence of which is shown in FIG. 4) was used to produce a replication-deficient retrovirus (RDR) that stably integrated the hLAL transgene into the genome of the founder (XLL109). This plasmid vector includes retroviral nucleotide sequences required for viral RNA packaging, reverse transcription and integration, but does not contain the intact sequences for the viral gag, pol and env genes. The methods used to generate the retroviral vector and their use in subsequent transgenesis procedures are described herein.

The retroviral portion of pALVIN-OVR1-I-hLAL-dSA is based on the ALV vector, pNLB. pNLB was modified such that the LTRs would be self-inactivating (SIN) (FIG. 3B). To accomplish this, 273 bp of the 3′ LTR was deleted, which includes the enhancer and CAAT box of the U3 region. Because the inactivated U3 region at the 3′ end of the retroviral sequence serves as a template for a new U3 region present at the 5′ end of an integrated provirus, 5′ LTR is normally also inactivated. The deletion of LTR sequences in the SIN construct decreases promoter interference on the internal promoter from the LTR, and minimizes the possibility for recombination of sequences to form a replication competent retrovirus. The new vector is termed pALVIN for ALV inactivation vector.

Downstream of the 5′ LTR are the partial gag and env coding sequences, which were carried over from the pNLB vector. In pALVIN-OVR1-I-hLAL-dSA, a small portion (12%) of the gag protein precursor sequence remains (55% of the p19 mature peptide sequence) and a small portion (1.7%) of the env precursor sequence of RAV2 remains (GenBank Accession, AF033808). These truncated gag and env regions are unable to produce functional proteins needed to create replication competent retrovirus (Cosset, 1991).

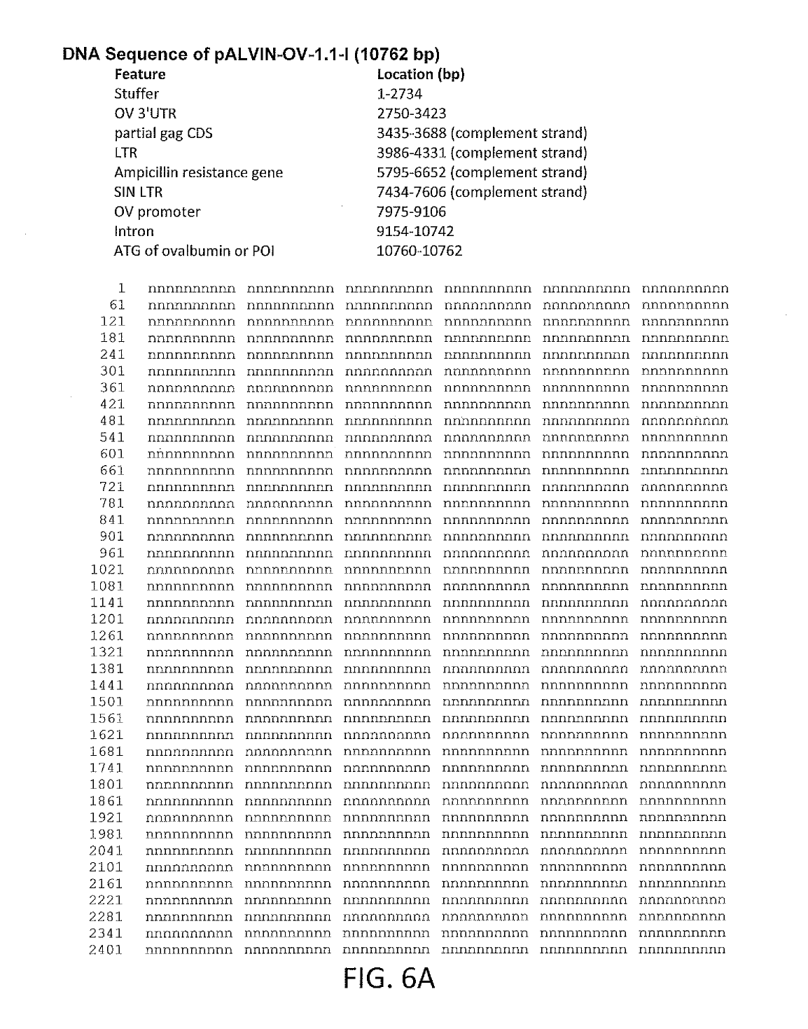

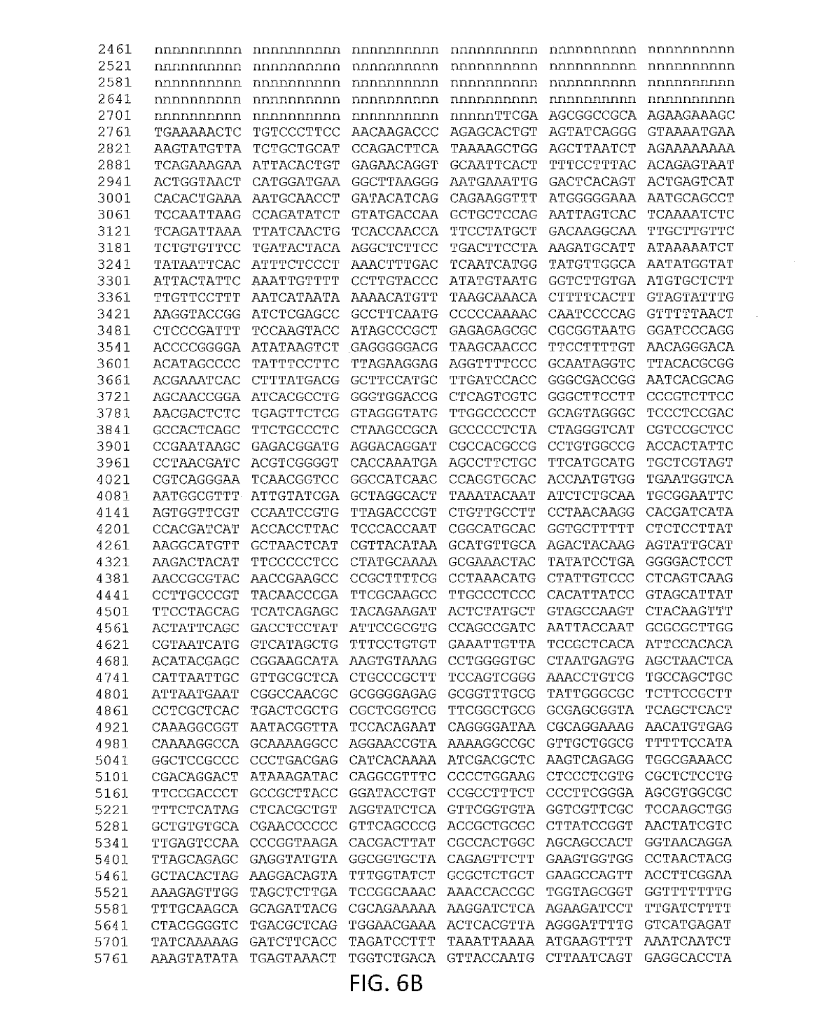

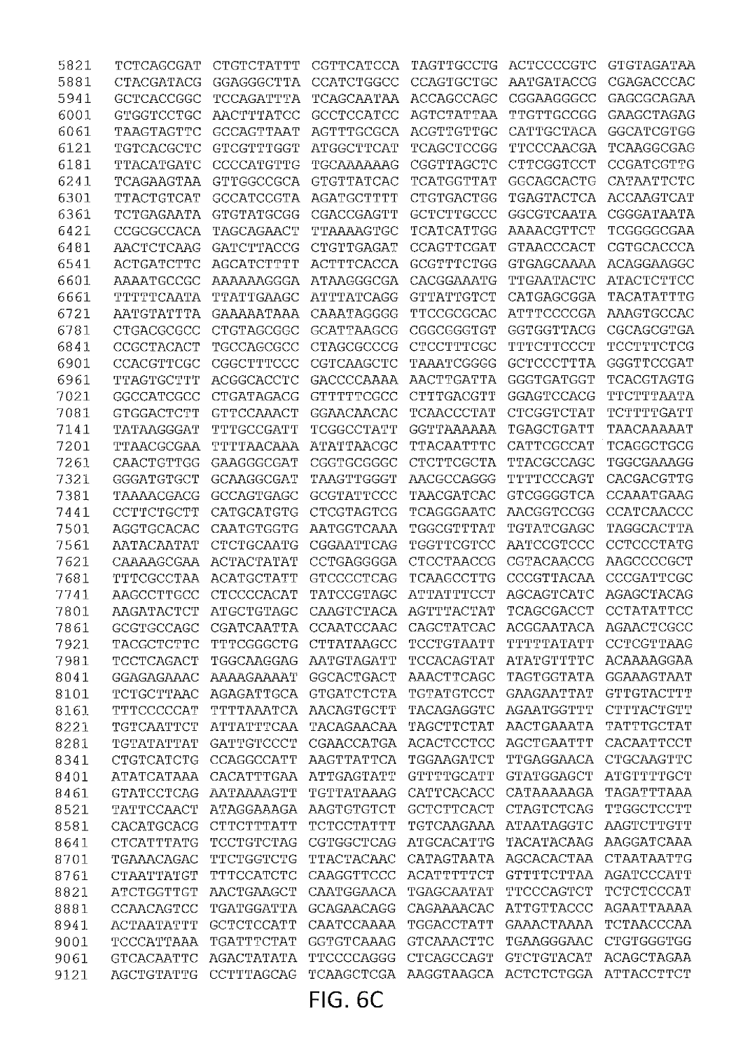

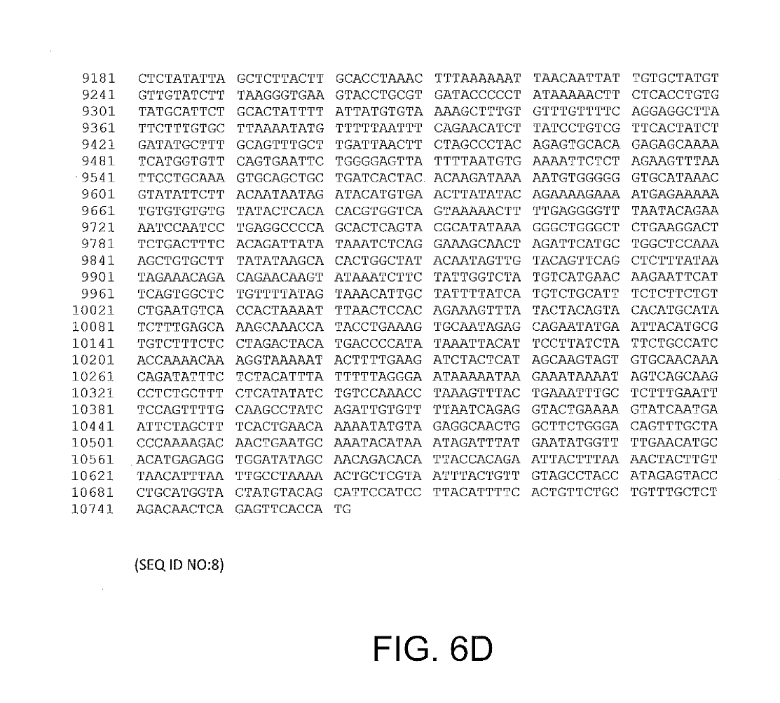

Transcriptional and translational control elements of the chicken ovalbumin gene were inserted into pALVIN to create pALVIN-OV-1.1-I (sequence of which is shown in FIG. 6; SEQ ID NO: 8). The first section of pALVIN-OV-1.1-I is composed of a contiguous section of the chicken ovalbumin gene which includes the 1.1 kb proximal promoter region, the first exon, first intron and part of the 2^{nd} exon. The next section is a stuffer insert fragment that takes the place of the ovalbumin protein coding sequences. The stuffer is followed by the 3′ untranslated region (UTR) of the chicken ovalbumin gene, which includes sequences that facilitate proper processing of the mRNA, including polyadenylation. In general, the stuffer fragment is replaced by DNA fragments encoding the desired protein, in this case hLAL. The result is a vector that has specific elements that promote regulated transcriptional expression and translation of an mRNA in the oviduct of transgenic chickens, that closely mimics regulation of the endogenous ovalbumin mRNA, and that allows high expression of the protein of interest in egg white.

The pALVIN-OV-1.1-I vector includes the first intron of the ovalbumin gene. Because the intron is susceptible to splicing during the production and packaging of the retroviral RNA genome, we inserted the expression cassette in the opposite orientation relative to the LTRs. In this way the intron is not recognizable in the retroviral RNA and is packaged without splicing. For convenience all maps in this document are drawn with the LTRs in the opposite orientation and the expression cassette in the forward or clockwise orientation.

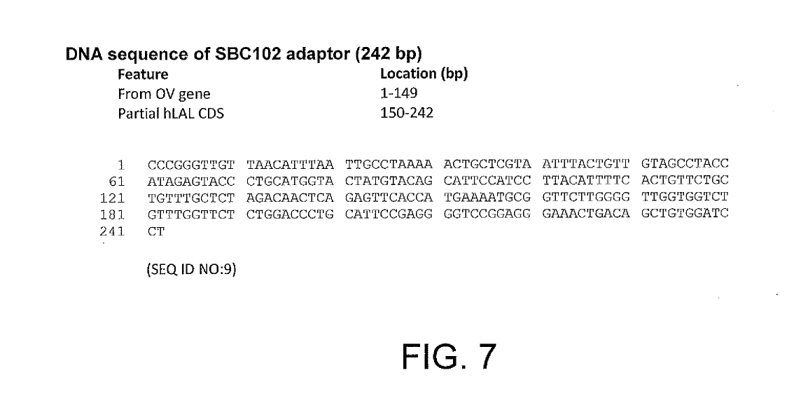

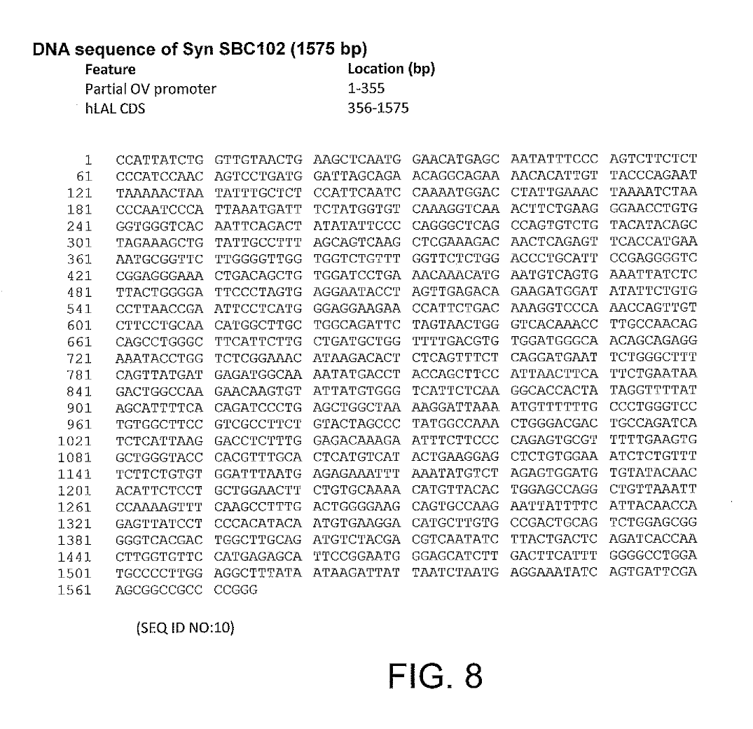

pALVIN-OV-1.1-I is the base vector into which the coding sequence (CDS) of hLAL was inserted. Two DNA fragments, hLAL adaptor and Syn hLAL, which make up the hLAL CDS and sequences required for compatibility with pALVIN-OV-1.1-I, were synthesized at Integrated DNA Technologies, Coralville, Iowa, (see FIGS. 7 and 8; SEQ ID NOs: 9 and 10). A 229 bp HpaI/BamHI fragment of hLAL adaptor and a 1113 bp BamHI/BstBI fragment of Syn hLAL were inserted into the 7882 HpaI/BstBI fragment of pALVIN-OV-1.1-I, thereby replacing the stuffer region with the hLAL CDS and creating pALVIN-OV-1.1-1-hLAL.

It was discovered that there was a cryptic splice site in the antisense strand of the hLAL CDS which prevented packaging of intact retroviral RNA. The cryptic splice site was removed by alteration of the DNA sequence without changing the amino acid sequence of hLAL. This change was performed by polymerase chain amplification of region 232 to 534 of pALVIN-OV-1.1-I-hLAL with primer 5′-AGAAACTGAGAGTGTCTTAT-3′ (SEQ ID NO: 12) and primer 5′-TGACAGCTGTGGATCCAGAAACAAACATG-3′ (SEQ ID NO: 13), creating a 329 bP amplicon. This amplicon was digested with BamHI and SexAI and ligated with the 8940 bP BamHI/SexAI fragment of pALVIN-OV-1.1-I-hLAL to create pALVIN-OV-1.1-I-hLAL-dSA.

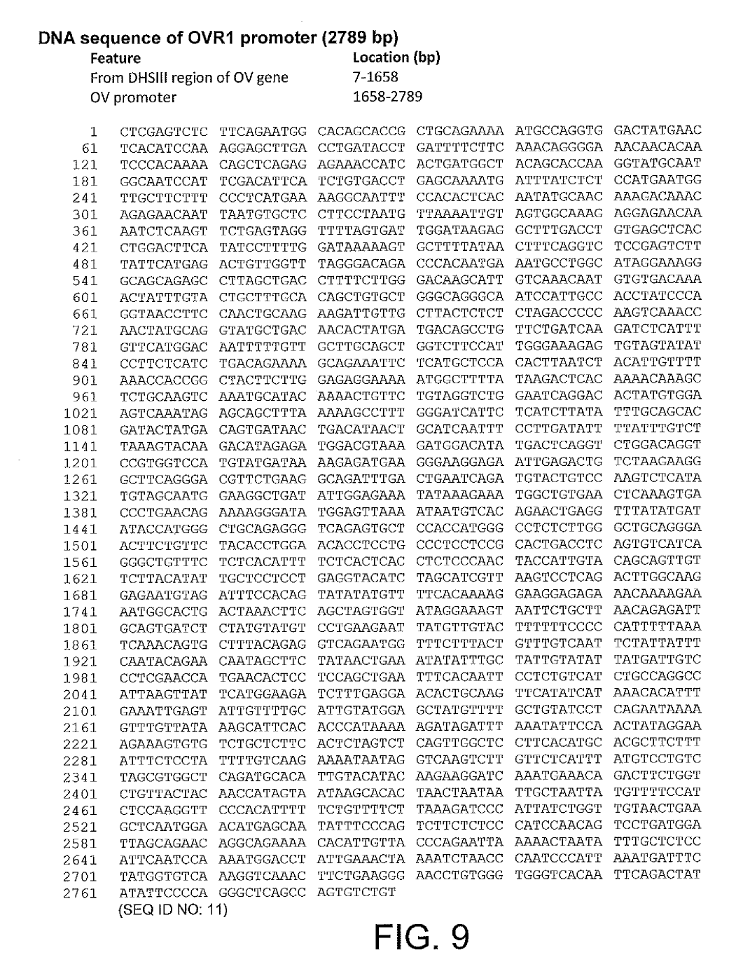

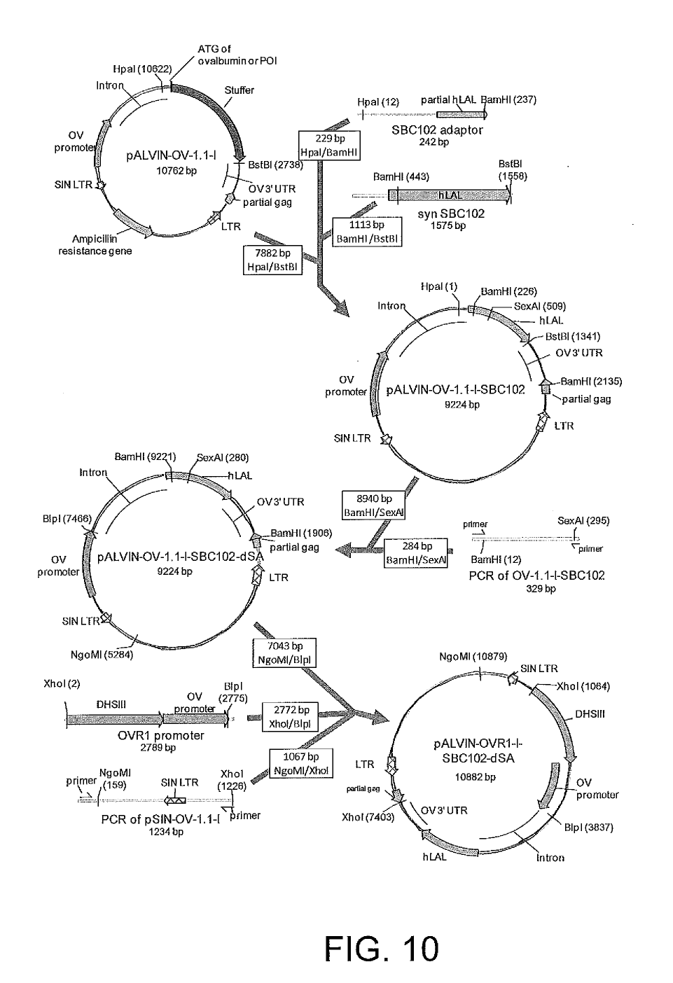

A putative promoter enhancer which contains DNase hypersensitive site III (DHSIII) of the chicken ovalbumin gene (−3819 to −2169 relative to the OV promoter start site) (Kaye, Bellard et al. 1984) was inserted into pALVIN-OV-1.1-I-hLAL-dSA to create pALVIN-OVR1-I-hLAL-dSA. This was performed as follows: a DNA fragment which included the DHSIII enhancer and 1.1 kb proximal OV promoter termed OVR1 promoter (see FIG. 9; and SEQ ID NO: 11 for sequence) was isolated by digestion with XhoI and BlpI. To facilitate subcloning, an adaptor fragment, PCR of pSIN-OV-1.1-1 was generated by PCR amplification of region 6752 to 7974 of pALVIN-OV-1.1-I with primers 5′-GCCGCTCGAGCGAGGAATATAAAAAAATT-3′ (SEQ ID NO: 14) and 5′-TCCGCGCACATTTCCCCGAA-3′(SEQ ID NO: 15) followed by digestion with NgoMI and XhoI. The 2772 bp XhoI/BlpI fragment of OVR1 promoter and 1067 bp NgoMI/XhoI fragment of PCR of pSIN-OV-1.1-I were inserted into the 7043 bp NgoMI/BlpI fragment of pALVIN-OV-1.1-I-hLAL-dSA, thereby creating pALVIN-OVR1-I-hLAL-dSA (see FIG. 10 for the construction schematics of pALVIN-OVR1-I-hLAL-dSA). The construction of the retroviral vector segment of the vector, denoted as pALVIN (aka pAVIJCR-A395.22.3.1-KM or pALV-SIN), is described in United States Patent Application 2008/0064862.

In addition, included is the production of LAL in accordance with the invention using a promoter and/or vector disclosed in US patent publication No. 2008/0064862, published Mar. 13, 2008, the disclosure of which is incorporated in its entirety herein by reference.

### Example 2

### Viral Particle Production

The G0 founder transgenic male, XLL109, carrying the hLAL transgene in its genome, was created by using a retroviral transgenesis method as follows. Replication-defective viral particles carrying the pALVIN-OVR1-I-hLAL-dSA vector were produced by transient transfection of an immortalized chicken fibroblast cell line. These chicken fibroblast cells were simultaneously transfected with three plasmids, pALVIN-OVR1-I-hLAL-dSA, pCMV-gag-pol and pCMV-VSV-G. pCMV-gag-pol expresses the gag and pol genes of RAV1 strain of the avian leukosis virus. pCMV-VSV-G expresses the envelope protein of the vesicular stomatitis virus. Four hours after transfection, the media was replaced with DMEM supplemented with 10% fetal bovine serum, 100 units/mL penicillin and 100 μg/mL streptomycin. Media was harvested at 48 hours post-transfection, filtered through a 0.45 micron filter (Millipore) and concentrated by ultracentrifugation. Concentrated retrovirus carrying the ALVIN-OVR1-I-hLAL-dSA transgene was collected and used in the transduction of early stage embryos. Note that because “p” is the notation for the plasmid form of vector, the “p” is absent from the transgene designations once the transgene is in the form of packaged vector or integrated transgene.

### Example 3

### Embryo Transgenesis

Integration of the ALVIN-OVR1-I-hLAL-dSA expression cassette into the genome of an embryo was achieved by transduction of early stage embryos (Speksnijder and Ivarie, 2000). Freshly laid fertilized White Leghorn eggs were obtained from a breeding colony. An aperture was made in the shell to provide access to the embryo. Seven microliters of concentrated replication deficient retrovirus particles carrying the ALVIN-OVR1-I-hLAL-dSA expression cassette described above were injected into the subgerminal cavity of the embryo. Eggs were sealed with hot glue, and then incubated and hatched under standard conditions. Progeny produced from these injections were given individual identification markers at hatch for identification and traceability. Blood samples from the progeny were transgene positive when analyzed by real-time PCR for the hLAL transgene using PCR primers specific for the hLAL coding sequence (as described below). This gave an indication that the transgenesis procedure was successful. The real-time PCR assay for the hLAL transgene utilizes Taqman® chemistry (Applied Biosystems). The forward and reverse primers were 5′-ACGACTGGCTTGCAGATGTCT-3′ (SEQ ID NO: 16) and 5′-CCCCAAATGAAGTCAAGATGCT-3′ (SEQ ID NO: 17), respectively. The Taqman® probe sequence was 5′-CCGGAATGCTCTCATGGAACACCAA-3′ (SEQ ID NO: 18) and was labeled with FAM (as the emitter) at the 5′ end and Iowa Black (as the quencher) at the 3′ end. Primers, probe and 1 μl of extracted DNA was added to 30 μl Taqman® Universal Master Mix (Applied Biosystems). Control reactions included various dilutions of a plasmid bearing the hLAL sequence and DNA from wild-type chickens (data not shown). Standard cycling parameters were used on an Applied Biosystems 7500 Fast Real-Time PCR System.

### Example 4

### Identification of G0 founder

Semen was collected from sexually mature males and DNA was extracted and assayed using the hLAL real-time PCR assay. The number of transgene copies in each sample was estimated using known standards (a plasmid bearing the hLAL gene) mixed with negative control semen DNA. The transgene cassette DNA content in male XLL109 was at a level that would allow transmission of the transgene to his progeny, as estimated by real-time PCR. This XLL109 male was the G0 transgenic founder and was bred with non-transgenic chickens to generate the G1 hemizygotic transgenic chickens.

### Example 5

### Propagation and Characterization of Hemizygotic G1 Avians

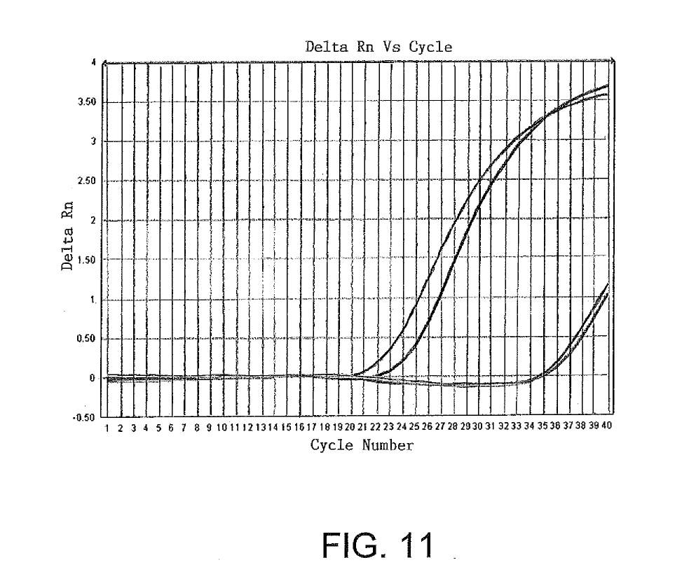

Progeny sired by the transgenic founder XLL109 were tested for the presence of the transgene in blood cell DNA using the hLAL real-time PCR assay. Blood was collected from 1-2 week old progeny and DNA was extracted using a high-throughput technique (Harvey et al., 2002). The DNA solutions were not quantified prior to the Taqman assay to facilitate the high-throughput screen. Typically 1 μl of DNA solution contains 50 to 400 ng of DNA which is sufficient to generate a positive amplification signal. A total of 1,322 chicks sired by XLL109 were tested, and positive progeny were re-bled and tested for confirmation. According to the PCR results, 22 progeny were positive for the ALVIN-OVR1-I-hLAL-dSA transgene. An example of the Taqman results is shown in FIG. 11.

### Example 6

### Identification and Characterization of High-Expression Line

One of the G1 chickens, 1LL7466, laid eggs with significantly higher levels of rhLAL protein in the egg white, as compared to the other G1 chickens. Southern blot analysis was performed on 1LL7466 and sibling G1 males to identify which sibling males had the same integration site as the high expressing chicken. Digests were performed with a restriction enzyme that cut only once within the transgene (BlpI), and the Southern blots were probed with a segment of the ovalbumin promoter or the hLAL coding sequence (FIGS. 12A-D). The position of the 2^{nd} restriction site, which resides in the flanking genomic region, varies depending on the site of integration. Thus the size of the BlpI band detected by the OV probe or hLAL probe is unique to each line generated.

The OV probe detected a single band of 4.1 kb in BlpI-digested DNA from wild-type chickens, which corresponded to the expected size of a BlpI segment within the endogenous ovalbumin gene of the chicken genome (FIGS. 12B and 12D). A second band of 4.3 kb was detected with chicken 1LL7466, which corresponded to the transgene band. Three additional female siblings, 1LL10409, 1LL10686 and 1LL12058 and three additional male siblings, 1LL8922, 1LL9330 and 1LL11217 displayed the 4.3 kb band, indicating that these siblings might be of the same line (FIGS. 12B and 12D).

As expected the hLAL probe did not detect a band in DNA from wild-type chickens as the DNA sequence of the chicken lysosomal acid lipase gene and the coding sequence for the recombinant human lysosomal acid lipase are sufficiently differentiated to not permit hybridization under the conditions used in these Southern assays (FIG. 12C). The hLAL probe detected a single band of ˜10.6 kb in BlpI-digested genomic DNA from the same chickens that were positive for the 4.3 kb band detected by the OV probe, indicating that these 7 G1 chickens have the same integration site and thus are of the same line.

No other bands were detected, indicating that 1LL7466, 1LL10409, 1LL10686, 1LL12058, 1LL8922, 1LL9330 and 1LL11217 all had a single integration site.

The Southern analysis also indicated that the transgene was integrated as the bands detected by the OV and hLAL probes were of different sizes and greater in size than from the transgene alone. A map showing the predicted structure of the integrated transgene and position of BlpI sites in the flanking genomic regions is shown in FIG. 12A.

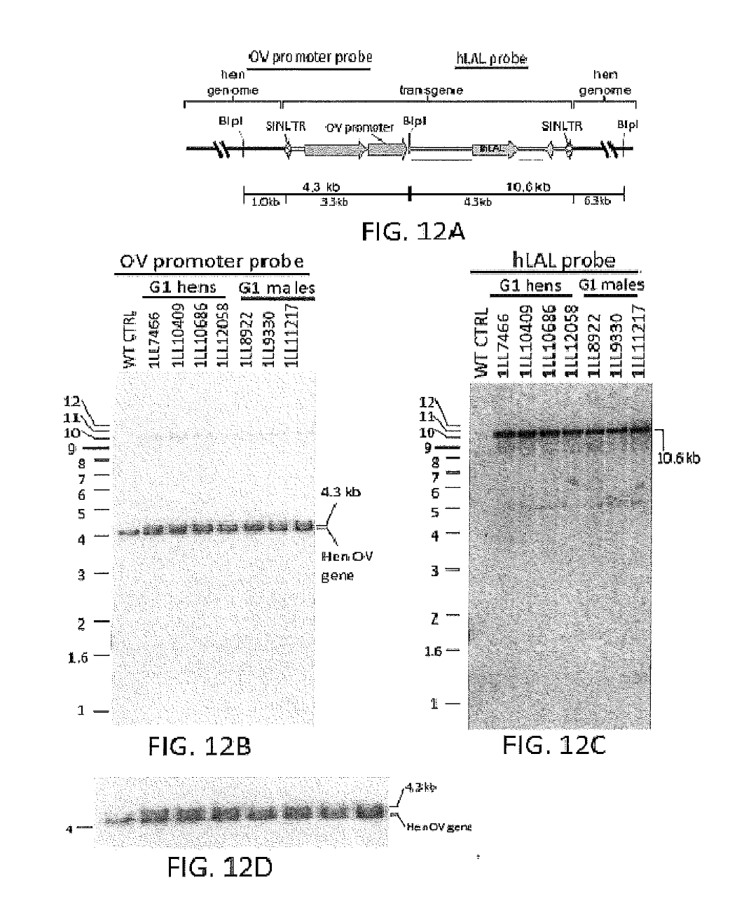

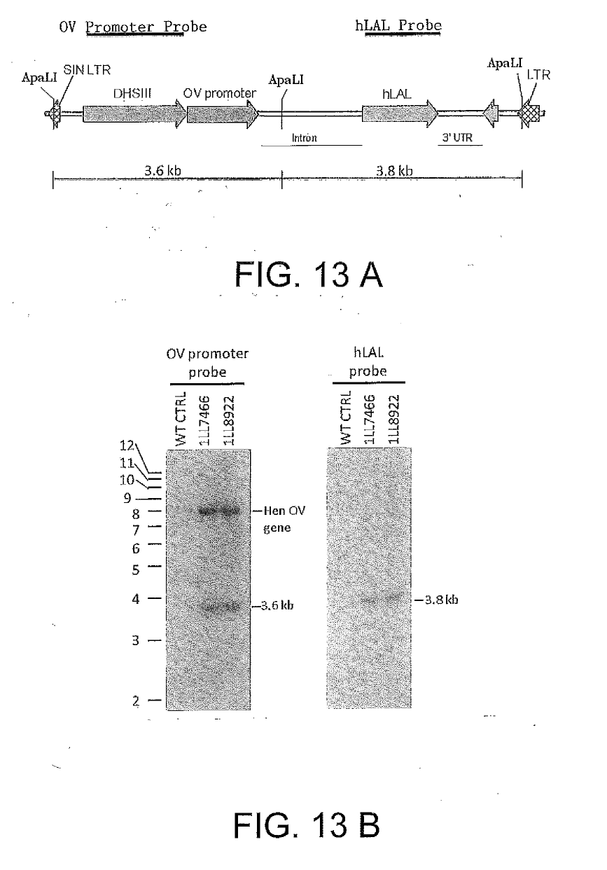

To confirm that the transgene is intact, two steps were taken. First, the hLAL coding sequence was isolated by PCR from 1LL7466. The PCR products were sequenced on both strands from the hLAL start codon to the stop codon. The DNA sequence was exactly as expected, indicating no changes in the DNA sequence of coding regions in the transgene. Second, Southern blot analysis was conducted using restriction enzyme ApaLI, which digests intact transgene into 2 segments, 3.6 and 3.8 kb (FIG. 13A). Both the 3.6 and 3.8 kb bands were detected in ApaLI-digested genomic DNA from G1s, indicating that the transgene was integrated in a fully intact form (FIG. 13B).

### Example 7

### Propagation and Characterization of G2s

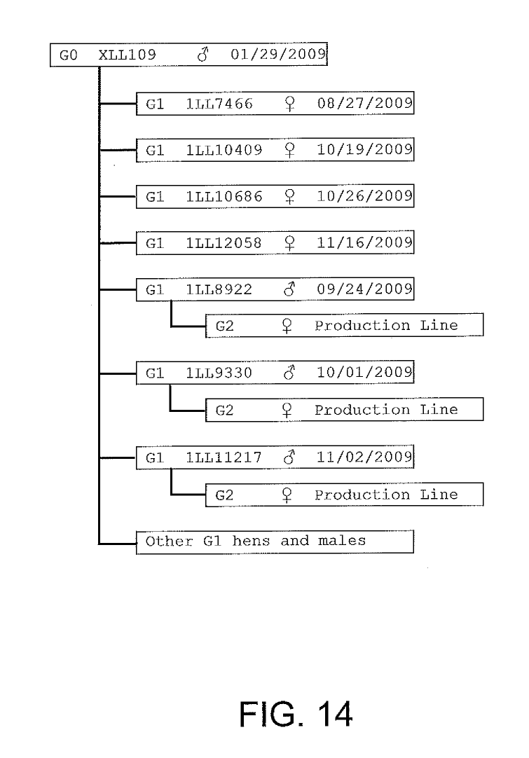

FIG. 14 shows the lineage of the hLAL G2s descended from a single G0 founder, XLL109. At the G1 stage, the transgene was characterized with regard to copy number, integrity, hLAL sequence and integration site—and seven G1 transgenics were identified and characterized (four chickens and three roosters). Propagation of the G2s was accomplished by artificial insemination of non-transgenic chickens with semen collected from the G1 sires 1LL8922, 1LL9330 and 1LL11217 (FIG. 14). Each inseminated chicken, her eggs and subsequent progeny were housed separately from the other progeny. Hatched progeny were tested for presence of the hLAL transgene using the hLAL real-time PCR assay. Because G1 founders were hemizygous with respect to the transgene, half of the progeny were expected to be transgenic G2s. Of 610 G2 progeny analyzed to date, 330 or 54% were transgenic.

### Example 8

### Genetic Analysis of the hLAL Avians

After identification of each G2 chicken by the hLAL real-time PCR assay of blood DNA, the production line is subjected to the following genetic assays: the hLAL gene was PCR-amplified from blood DNA and sequenced to confirm 100% homology with the human sequence; the transgene integration site was confirmed by integration site PCR, as described above. The PCR sequencing and integration site analysis was performed on: each chicken in a <10 chicken production line; 10% of chickens (minimum 10) for 11-100 chicken production line; 5% of chickens (minimum 10) for 101-1000 chicken production line; 1% of chickens (minimum 50) for 1001-10,000 chicken production line; 0.1% of chickens (minimum 100) for >10,001 chicken production line. Detailed records were maintained at every step of the growing and production phase.

### Example 9

### Purification of hLAL from Egg White

Egg white (EW) containing LAL was solubilized at pH 6 overnight and clarified through centrifugation (or depth filtration) with 0.2 um filtration. The EW was adjusted with 1 M NaOAc buffer (pH 4) to pH 6.

The clarified EW was loaded onto a Phenyl-HIC column (EW:column size=2:1) equilibrated with 20 mM phosphate/137 mM NaCl buffer (pH 6). After the completion of loading, the column was washed with equilibration buffer and 5 mM phosphate buffer (pH 6). The LAL was eluted with 30% propylene glycol with 5 mM Tris buffer (pH 7.2).

The eluted LAL fraction was adjusted to pH 5 with 1 M acid and then loaded onto a GigaCap S column (EW:column size=10:1). The column was equilibrated with 50 mM NaOAc buffer (pH 5). After completion of loading, the column was washed with the equilibration buffer. The LAL was eluted with 50 mM NaOAc/60 mM NaCl (pH 5).

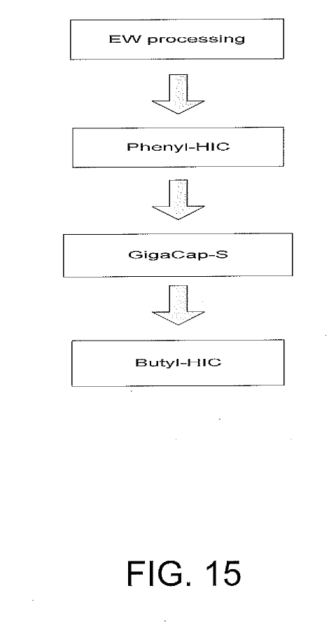

The LAL fraction off the GigaCap S column was adjusted to pH6 with 1 M Tris buffer and then loaded onto a Butyl-HIC column (EW:column size=10:1). The column was equilibrated with 20 mM phosphate/137 mM NaCl buffer (pH 6). After the completion of loading, the column was washed with equilibration buffer and 5 mM phosphate buffer (pH 6). The pure LAL was eluted with 50% propylene glycol with 5 mM Tris buffer (pH 7.2). FIG. 15 depicts the purification steps of hLAL from egg white.

### Example 10

### Carbohydrate Analysis of Transgenic Avian Derived hLAL

The oligosaccharide structures were determined for avian derived human LAL by employing the following analysis techniques as are well known to practitioners of ordinary skill in the art.

Two hundred micrograms were digested with trypsin and chymotrypsin for 18 h at 37° C. in 0.1 M Tris-HCl, pH 8.2, containing 1 mM CaCl₂. The digestion products were enriched and freed of contaminants by Sep-Pak C18 cartridge column. After enrichment, the glycopeptides were digested with 2 μl of PNGaseF (7.5 unit/ml) in 50 μl of 20 mM sodium phosphate buffer, pH 7.5, for 18 h at 37° C. Released oligosaccharides were separated from peptide and enzyme by passage through a Sep-Pak C18 cartridge column.

The glycan fraction was dissolved in dimethylsulfoxide and then permethylated based on the method of Anumula and Taylor (Anumula and Taylor, 1992). The reaction was quenched by addition of water and per-O-methylated carbohydrates were extracted with dichloromethane. Per-O-methylated glycans were dried under a stream of nitrogen.

MALDI/TOF-MS (Matrix assisted laser desorption ionization time-of-flight mass spectrometry) was performed in the reflector positive ion mode using α-dihydroxybenzoic acid (DHBA, 20 mg/mL solution in 50% methanol:water) as a matrix. All spectra were obtained by using a Microflex LRF (Broker).

MALDI-TOF-MS analysis and ESI MS/MS (electrospray ionization tandem mass spectrometry) were performed on the oligosaccharides after release from the peptide backbone and purification as is understood in the art. Samples of the individual polysaccharide species were also digested with certain enzymes and the digest products were analyzed by HPLC as is understood in the art.

It is believed that there are about six N-linked glycosylation sites present on human LAL. See, Zschenker, et al (2005) J. Biochem., Vol 137, p 387-394, the disclosure of which is incorporated in its entirety by reference This reference also indicates that there may be an O-linked glycosyation site on Human LAL. The N-linked oligosaccharide structures identified are shown in FIG. 16.

The data revealed that many or all of these structures were found as an N-linked Glycosylation structure in LAL produced in accordance with the invention (FIG. 16). For example, A-n is found attached to LAL produced in accordance with the invention. For example, O-n is found attached to LAL produced in accordance with the invention. For example, at least one of B-n, C-n and D-n is found attached to LAL produced in accordance with the invention. For example, at least one of E-n and F-n is found attached to LAL produced in accordance with the invention. For example, at least one of I-n and J-n is found attached to LAL produced in accordance with the invention. For example, at least one of K-n and L-n is found attached to LAL produced in accordance with the invention. For example, at least one of M-n and N-n is found attached to LAL produced in accordance with the invention. For example, G-n is found attached to LAL produced in accordance with the invention. For example, H-n is found attached to LAL produced in accordance with the invention.

### Example 11

### N-Glycan Species of Transgenic Avian Derived LAL

Purified samples of transgenic avian derived hLAL (600 μg/sample) were dialyzed using a Tube-O-Dialyzer (4.0 kDa cut-off membrane; G Biosciences) against nanopure water at 4° C. for about 24 hours to remove salts and other contaminants. Nanopure water was replaced four times during the entire dialysis period.

After dialysis, each of the samples was divided into three aliquots: ˜¼ of sample weight for neutral and amino sugars analysis, ˜¼ of sample weight for mannose-6-phosphate analysis, and ˜½ of sample weight for oligosaccharide profiling. The aliquot intended for neutral and amino sugars analysis was hydrolyzed with 2 N trifluoroacetic acid (TFA) at 100° C. for 4 hours and the aliquot for mannose-6-phosphate analysis was hydrolyzed with 6.75 N TFA at 100° C. for 1.5 hours. The hydrolysates were then dried under N₂, redissolved with 50 μl H₂O, sonicated for 7 min in ice and transferred to an injection vial. However, the neutral and amino sugar samples were diluted 2 times because the peaks produced from the originally dissolved hydrolysates were too large.

A mix of standards for neutral and amino sugars, and for mannose-6-phosphate with a known number of moles was hydrolyzed in the same manner and at the same time as the sample. Four concentration of the neutral and amino sugar standard mix (Fuc & GalNAc, 0.2, 0.4, 0.8, and 1.6 nmoles per 10 μL; GlcNAc, 0.5, 1.0, 2.0, and 4.0 nmoles per 10 μL; Gal & Man, 0.3, 0.6, 1.2, and 2.4 nmoles per 10 μl; and Glc, 0.1, 0.2, 0.4, and 0.8 nmoles per 10 μL) and mannose-6-phosphate (640, 1280, 2560, 5120 picomoles per 10 μL) were prepared to establish a calibration equation. The number of moles of each sugar in the sample was quantified by linear interpolation from the calibration equation.

The neutral and amino sugars and mannose-6-phosphate were analyzed by HPAEC using a Dionex ICS3000 system equipped with a gradient pump, an electrochemical detector, and an autosampler. The individual neutral and amino sugars, and mannose-6-phosphate were separated by a Dionex CarboPac PA20 (3×150 mm) analytical column with an amino trap. The gradient programs used eluents A, degassed nanopure water and B, 200 mM NaOH for neutral and amino sugars, and C, 100 mM NaOH and D, 1 M sodium acetate in 100 mM NaOH for mannose-6-phosphate. Injection (10 μL/injection) was made every 40 minutes for neutral and amino sugar determination and every 35 minutes for mannose-6-phosphate determination. All methods were based on protocols described by Hardy and Townsend (Hardy, M. R., and Townsend, R. R., “High-pH anion-exchange chromatography of glycoprotein-derived carbohydrates”, 1994, Methods Enzymol. 230: 208-225). Instrument control and data acquisition were accomplished using Dionex chromeleon software. Results are shown in Table 1 below. The control sample is ovomucoid purified from EW.

TABLE 1
Monosaccharide composition of control and LAL by HPAEC.

| | | | nanomoles/ | mole |
|---|---|---|---|---|
| Sample ID | Analyte | nanomoles | μg | % |
| Control | Fucose | nd | — | — |
| | N-acetyl galactosamine | 5.066 | 0.020 | 9.6 |
| | N-acetyl glucosamine | 26.947 | 0.108 | 51.4 |
| | Galactose | 3.876 | 0.016 | 7.4 |
| | Glucose | nd | — | — |
| | Mannose | 16.565 | 0.066 | 31.6 |
| | Mannose-6-phosphate | nd | — | — |
| | N-acetyl neuraminic acid | ndm | — | — |
| | N-glycolyl neuraminic | ndm | — | — |
| | acid | | | |
| Transgenic | Fucose | nd | — | — |
| Avian | N-acetyl galactosamine | nd | — | — |
| derived | N-acetyl glucosamine | 17.932 | 0.120 | 37.6 |
| hLAL | Galactose | 0.879 | 0.006 | 1.8 |
| | Glucose | nd | — | — |
| | Mannose | 23.290 | 0.155 | 48.8 |
| | Mannose-6-phosphate | 5.642 | 0.038 | 11.8 |
| | N-acetyl neuraminic acid | ndm | — | — |
| | N-glycolyl neuraminic | ndm | — | — |
| | acid | | | |

nd = not detected;
ndm = not determined.

### Structural Features of LAL

LAL has 6 potential sites in its amino acid sequence for N-linked glycosylation, Asn³⁶, Asn⁷², Asn¹⁰¹ Asn¹⁶¹, Asn²⁷³, and Asn³²¹. Five of these, Asn³⁶, Asn¹⁰¹ Asn¹⁶¹, Asn²⁷³ and Asn³²¹ were found to be glycosylated while Asn⁷² was unglycosylated or substantially unglycosylated (substantially unglycosylated means in a mixture of LAL molecules, fewer Asn⁷² are glycosylated than any of Asn³⁶, Asn¹⁰¹ Asn¹⁶¹, Asn²⁷³ and Asn³²¹). Accordingly, one aspect of the invention is LAL (e.g., human LAL) which is unglycosylated and/or substantially unglycosylated at Asn⁷², and production and use of such LAL. However, LAL having a glycosylated Asn⁷² is within the scope of the invention. The N-glycan structures primarily consist of a mixture of bi-, tri- and tetraantennary structures with N-acetylglucosamine, mannose and mannose-6-phosphate (MGP) as the major sugars. Each site appears to have a favored set of structures (Table 2 and FIG. 17) which is one aspect of the invention. For example, M6P-modified N-glycans reside at least at Asn¹⁰¹ Asn¹⁶¹ and Asn²⁷³. The non-phosphorylated structures are typical of N-glycans found on endogenous egg white proteins. No O-linked glycans were detected as determined by lack of N-acetylgalactosamine (GalNac). No sialic acid was detected which is consistent with previously determined N-glycan structures of other endogenous and exogenous proteins produced in accordance with the invention. The invention includes LAL glycosylated with one or more of the oligosaccharide structures disclosed herein.

TABLE 2
Site residence of LAL glycan structures as
determined by LC/MS of glycopeptides.

| Site | Glycan structure |
|---|---|
| Asn³⁶ | GlcNAc4Man3GlcNAc2 |
| | Hex1GlcNAc4Man3GlcNAc2 |
| Asn⁷² | None detected |
| Asn¹⁰¹ | Phos2Man7GlcNAc2 |
| Asn¹⁶¹ | Phos1Man6GlcNAc2 |
| | GlcNAc1Phos1Man6GlcNAc2 |
| | Man3GlcNAc2 |
| | GlcNAc2Man3GlcNAc2 |
| | GlcNAc3Man3GlcNAc2 |
| | GlcNAc4Man3GlcNAc2 |
| | Hex1GlcNAc4Man3GlcNAc2 |
| Asn²⁷³ | Man7GlcNAc2 |
| | Man8GlcNAc2 |
| | Man9GlcNAc2 |
| | Phos1Man8GlcNAc2 |
| | Phos1Man9GlcNAc2 |
| Asn³²¹ | GlcNAc2Man3GlcNAc2 |
| | GlcNAc3Man3GlcNAc2 |
| | GlcNAc4Man3GlcNAc2 |
| | Hex1GlcNAc4Man3GlcNAc2 |
| | GlcNAc5Man3GlcNAc2 |
| | Hex1GlcNAc5Man3GlcNAc2 |
| | GlcNAc6Man3GlcNAc2 |
| | Hex1GlcNAc6Man3GlcNAc2 |

Hex, galactose;
Phos, phosphate;
Man, mannose;
GlcNAc2, N-acetylglucosamine

### Methods

Monosaccharide composition, including the neutrals, amino and M6P, was determined qualitatively and quantitatively by high pH anion exchange chromatography-pulsed amperometric detection (HPAEC-PAD).

The structures of the predominant glycans were determined with data from several mass spectrometry methods (MALDI-TOF, NSI-MS/MS and glycopeptide LC-MS).

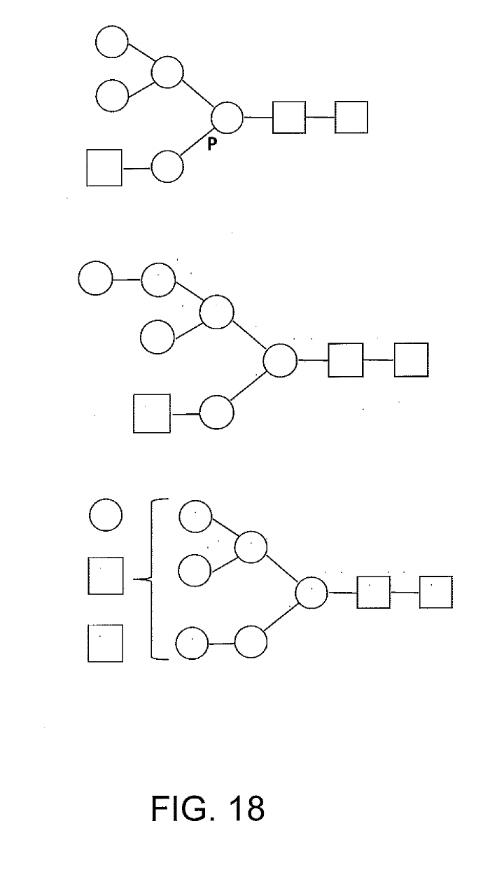

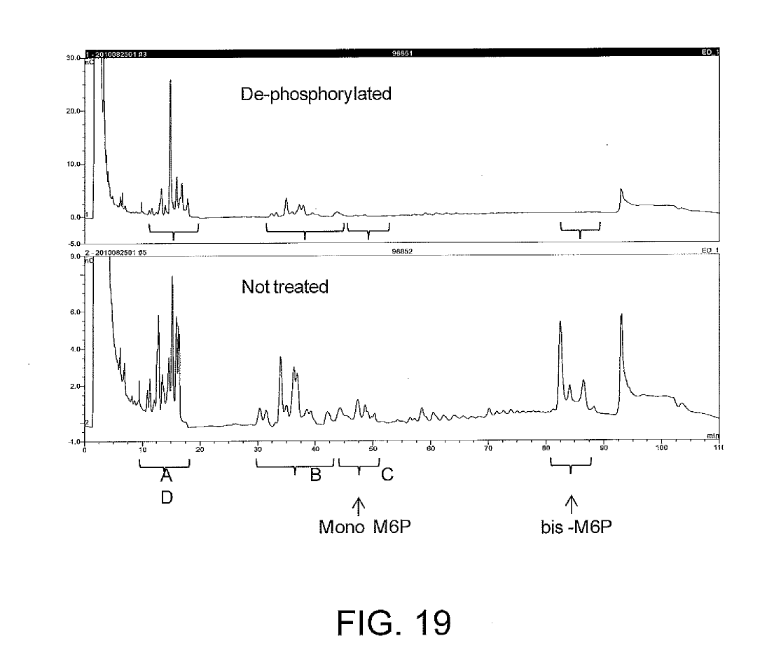

MALDI-TOF was useful for determination of neutral N-glycans and was able to detect phosphorylated N-glycans (FIG. 18). NSI-MS/MS was employed to determine the nature of minor peaks in the MALDI-TOF spectra, some of which were attributed to phosphorylated N-glycans (FIG. 19). Efforts to improve the ability of MALDI-TOF to detect phosphorylated N-glycans were not fruitful.

LC/MS of glycopeptides was able to detect neutral and phosphorylated structures and was able to determine the position of specific structures in the amino acid sequence of LAL (data summarized in FIG. 17 and Table 2).

To determine which peaks in the HPAEC-PAD chromatogram are due to phosphorylated N-glycans, LAL was treated with phosphatase and analyzed (FIG. 3). Peaks in groups C and D decreased in area under the curve (AUC) while a peak in group A became more prominent. Peaks in group B did not change in proportion to the other peaks. Based on the knowledge that retention time is proportional to the degree of charge (either due to phosphorylation or sialylation), it is contemplated that group C is composed of N-glycans with one phosphate (mono M6P) and group D composed of N-glycans with two phosphates (bis-M6P).

The retention time was also affected by composition and relative structural position of the neutral and amino monosaccharides. Such examples include the presence of galactose, the presence of a bisecting GlcNac and the degree of GlcNac substitution. Such factors contribute to the multiplicity of peaks in the HPAEC-PAD chromatogram.

### Example 12

### In Vitro Enzyme Activity Analysis of Transgenic Avian Derived hLAL in Egg White

Activity of Lysosomal Acid Lipase in egg white was determined using the fluorogenic substrate 4-methylumbelliferyl-oleate assay essentially as described in Yan et al. (2006), American Journal of Pathology, Vol. 169, No. 3, p 916-926, the disclosure of which is incorporated in its entirety herein by reference.

A stock solution of 4-methylumbelliferyl oleate (4-MUO) was prepared consisting of 2.5 mM 4-MUO in 4% Triton X-100. The assay was performed in a microtiter plate each well containing 62.5 μl of 0.2 M Sodium Citrate (pH 5.5) in 0.01% Tween80, 12.5 pI of egg white sample and 25 μl of the 2.5 mM 4-MUO. Change in fluorescence was monitored for 30 minutes at 37° C. using a Bio-Tek Synergy HT fluorometric microplate reader (excitation 360 nm and emission 460 nm). Prior to assay, egg white containing the hLAL was diluted to an enzyme concentration that resulted in the reaction continuing linearly for at least 30 minutes. The reaction was stopped with 50 μl of 0.75 M Tris-HCl, pH 8.0 and the endpoint fluorescence signal was measured in the same plate reader used above (excitation 360 nm and emission 460 nm).

Units of activity were determined using 4-methylumbelliferyl as a standard. One unit (U) is defined as the amount of enzyme which results in the formation of 1 umole of 4-methylumbelliferyl/min under the assay conditions described above. Non-hLAL containing egg white was used as a negative control.

Egg white samples which were positive for hLAL contained between 1 U and 100 U of activity per ml egg white. Egg white from 21 G1 chickens was analyzed. Egg white from 10 of the chickens tested positive for hLAL activity.

### Example 13

### In Vitro Analysis of Transgenic Avian Derived LAL

The ability of LAL produced in the oviduct cells of transgenic avians (referred to herein as “SBC-102,” “avian derived LAL,” “LAL,” or “hLAL”) to bind to cells and be internalized to the lysosomal compartment, was examined in vitro using macrophage and fibroblast cells. When incubated with macrophage cells, fluorescently-labeled SBC-102 was found to localize to the lysosome. This effect could be attenuated by using a mannose polysaccharide competitor, implicating the N-acetylglucosamine/mannose (GlcNAc/mannose) receptor as a mechanism of recognition and uptake by these cells. SBC-102 increased the cell-associated LAL activity in both LAL-deficient human fibroblasts and normal murine fibroblasts after incubation in vitro, indicating that exposure to SBC-102 can result in substantial replacement of deficient enzymatic activity.

Mannose-6-phosphate (M6P) is present in the oligosaccharide structures of SBC-102 which have been shown to be involved in the delivery of lysosomal enzymes to a wide variety of cells types via the ubiquitous M6P receptor.

LAL was purified from the egg white of transgenic hens. Oregon Green NHS was obtained from Invitrogen™ (#0-10241). The rat alveolar macrophage line, NR8383, and the mouse fibroblast line, NIH-3T3, were obtained from ATCC. LAL-deficient Wolman's fibroblasts were obtained from Coriell Institute for Medical Research and LysoTracker® Red was obtained from Invitrogen™.

Enzyme Labeling:

4 mg of transgenic avian derived LAL in PBS was labeled with Oregon Green, according to the manufacturer's recommendations and reaction was subsequently dialyzed against PBS then concentrated.

Macrophage Uptake:

Fluorescently-labeled transgenic avian derived LAL (5 μg/mL) and LysoTracker® Red were incubated with NR8383 cells for 2 hours. Cells were examined by co-focal fluorescence microscopy using a sequential scanning mode at 488 nm and then 514 nm.

Competitive Inhibition with Mannan:

Fluorescently-labeled SBC-102 (5 μg/mL) and mannan were incubated with NR8383 cells for 2 hours. Cells were trypsinized and LAL uptake measured by florescence-activated cell sorting using median fluorescence intensity as the endpoint.

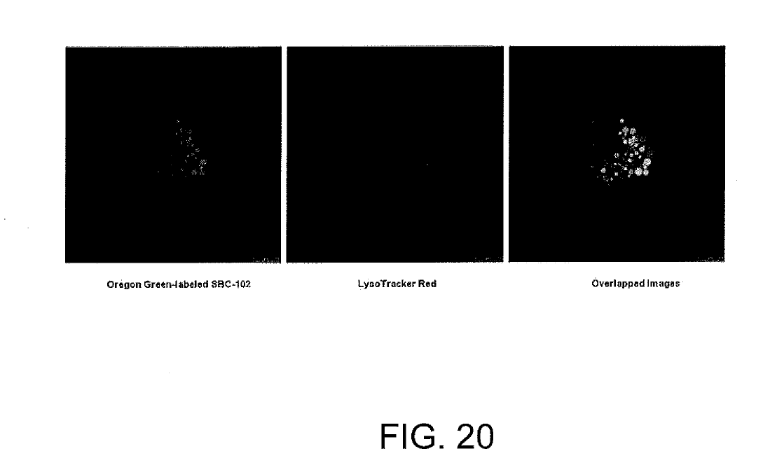

The ability of transgenic avian derived LAL to be taken up and subsequently incorporated into the lysosomes of target cells was examined using the macrophage cell line, NR8383. Fluorescently-labeled transgenic avian derived LAL and the lysosomal marker, “LysoTracker® Red” (Invitrogen™), were incubated with cells for 2 hours. The co-localization of transgenic avian derived LAL and lysosomal marker in the lysosomes of these cells was subsequently examined by confocal fluorescence microscopy using a sequential scanning mode (FIG. 20). The LAL demonstrated localization to lysosomes, which is consistent with similar in vitro studies using rhLAL from a variety of sources.

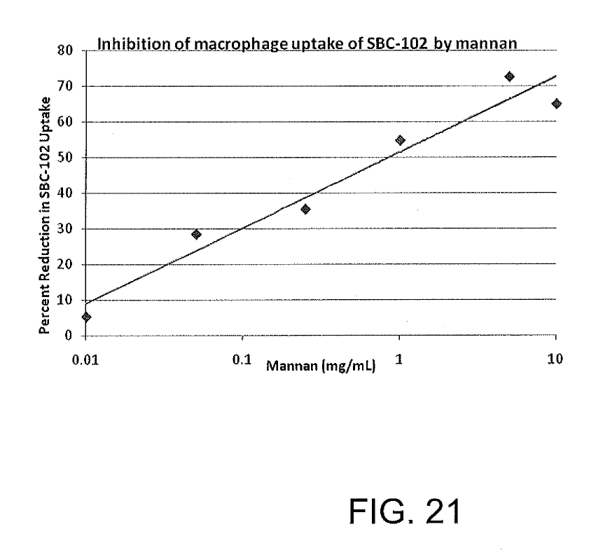

The binding specificity of transgenic avian derived LAL to the GlcNAc/mannose receptor has been assessed by competitive binding assays using the macrophage cell line, NR8383 (FIG. 21). Fluorescently-labeled (Oregon Green) transgenic avian derived LAL at 5 pg/mL and various concentrations of the mannose-containing oligosaccharide, mannan, were co-incubated with cells for 2 hours. The relative inhibition of transgenic avian derived LAL uptake by mannan, as compared with no mannan control, was quantified by fluorescence-activated cell sorting analysis using median fluorescence intensity as the endpoint. A mannose dose dependent inhibition in transgenic avian derived LAL binding/uptake was observed, which is consistent with transgenic avian derived LAL:GlcNAcR interaction.

In addition, mannose-6-phosphate mediated uptake in fibroblast cells was demonstrated by competition experiments with mannose-6-phosphate (results not shown).

The ability of transgenic avian derived LAL exposure to increase LAL activity in cells has been examined using both normal and LAL-deficient cells in vitro.

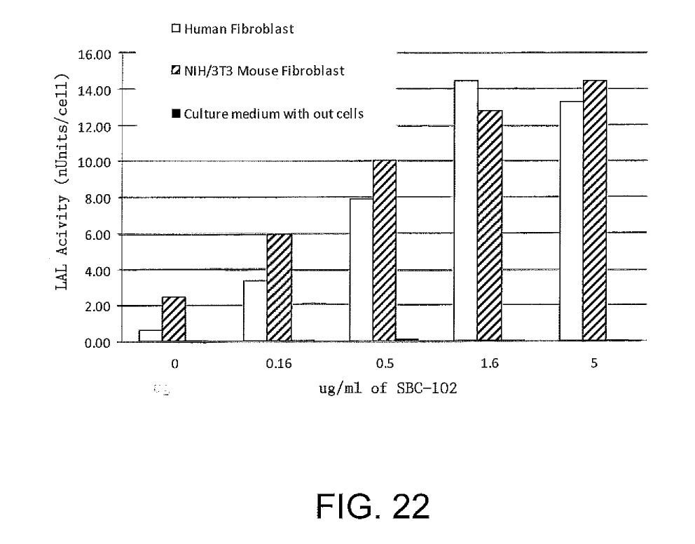

Fibroblasts isolated from a Wolman's patient and normal murine fibroblasts (NIH-3T3) were incubated in the presence of transgenic avian derived LAL at concentrations of either 0, 0.16 or 0.5 μg/mL for 5 hours. Cells were then washed to remove non-specific signal and cell lysates were assayed for LAL activity using 4-MUO substrate. Endogenous cell-associated LAL activity was lower in Wolman's fibroblasts compared to NIH-3T3 and dose-dependent increases in activity were observed in both cell types after incubation with transgenic avian derived LAL (FIG. 22).

### Example 14

### In Vivo Analysis of Transgenic Avian Derived LAL

LAL-deficient Yoshida Rats (i.e., Homozygous) (see Kuriyama et al. (1990), Journal of Lipid Research, vol. 31, p 1605-1611; Nakagawa et al., (1995) Journal of Lipid Research, vol. 36, p 2212-2218; and Yoshida and Kuriyama (1990) Laboratory Animal Science, vol. 40, p 486-489) were treated with either SBC-102 (5 mg/kg, IV) or placebo, once/week for four weeks beginning at four weeks of age. For each administration the SBC-102 was injected into the rat tail vein in two equal doses (2.5 mg/kg) 30 minutes apart. Rats and aged-matched wild-type controls were examined one week after the final dose. Analyses were done in triplicate.

Gross pathologic examination of the SBC-102 treated animals demonstrated normalization in liver color in addition to reduction in organ size. The SBC-102 treated rats showed essentially normal liver histology in marked contrast to the substantial accumulation of foamy macrophages in the vehicle-treated animals (data not presented). Serum alanine and aspartate transferase levels, which are elevated in LAL^{-/-} rats, were also reduced in SBC-102 treated rats (not shown).

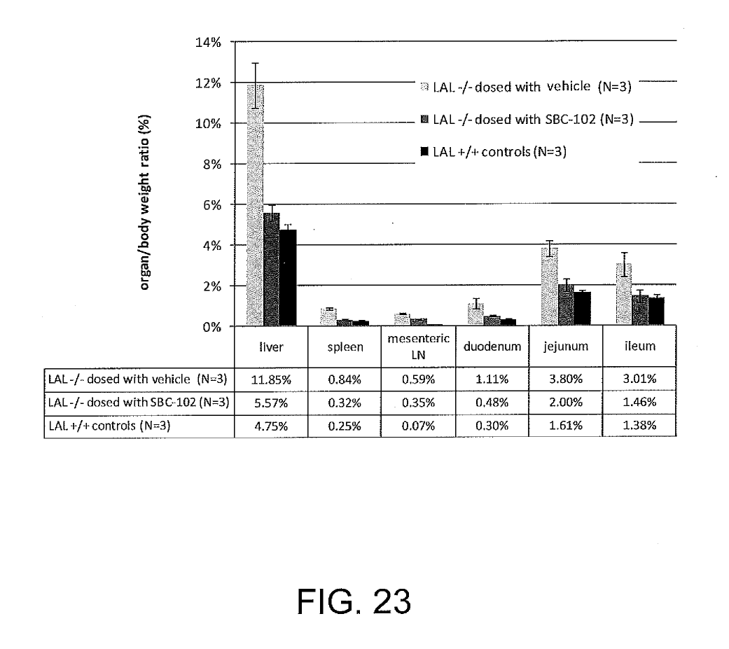

Mass of internal organs and tissue was determined for each rat and the data is shown in FIG. 23. Organ size is represented as percent of body weight determined at 8 weeks of age, in LAL^{-/-} rats and LAL^{+/+} rats after weekly administration of vehicle or SBC-102 at 5 mg/kg for 4 weeks.

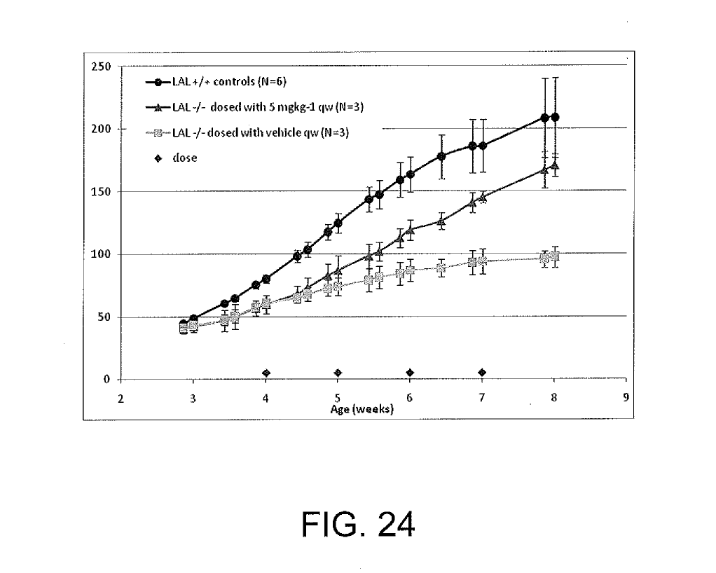

Body weight of SBC-102- or vehicle-treated Yoshida rats were compared with wild type rats, as is shown in FIG. 24. SBC-102 (5 mg/kg) or vehicle was administered by IV injection either as a single dose or as split doses (given within 4 hour period) to LAL^{-/-} rats. LAL^{+/+} rats are age-matched littermate controls.

### Example 15

### Triglyceride Analysis

Triglyceride analysis was performed on liver and spleen tissue from wild type, homozygous placebo and homozygous SBC-102 treated animals. The triglyceride analyses were performed using standard methodologies (i.e., MBL International's Triglyceride Quantification Kit Catalog # JM-K622-100) and were done in triplicate.

TABLE 3
Liver and Spleen Triglyceeride levels in wild-type
and LAL deficient rats
Triglyceride (ug/mg wet tissue)

| | Wild Type | Placebo | SBC-102 |
|---|---|---|---|
| | (n = 3) | (n = 3) | (n = 3) |
| Liver | 48 | 84 | 57 |
| Spleen | 3 | 22 | 4 |

### Liver Substrate Levels

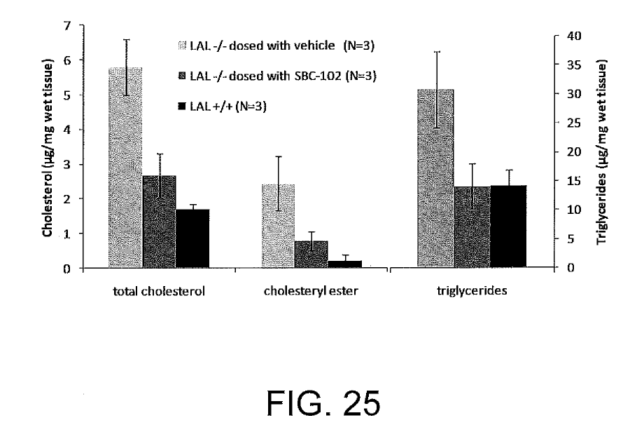

FIG. 25 shows liver cholesterol, cholesteryl ester and triglyceride levels determined at 8 weeks of age, in WT and LAL deficient rats after weekly administration of vehicle or SBC-102 at 5 mg·kg⁻¹ for 4 weeks.

### Example 16

### Dose Response Study

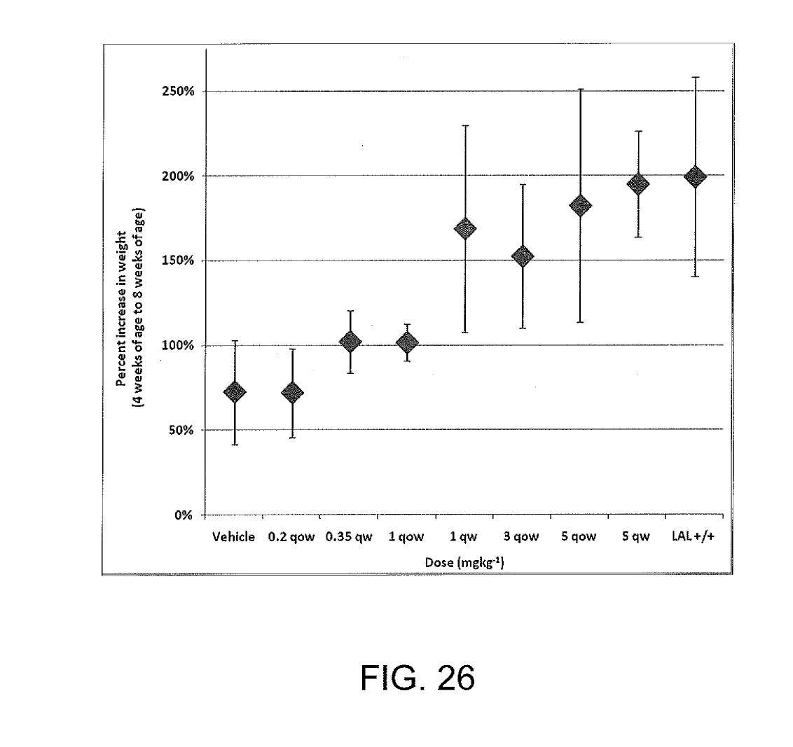

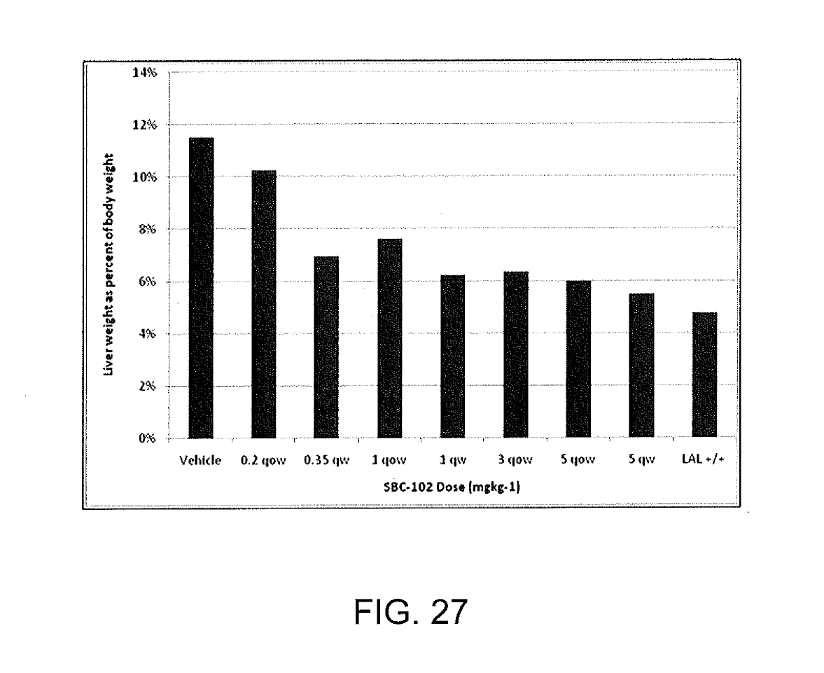

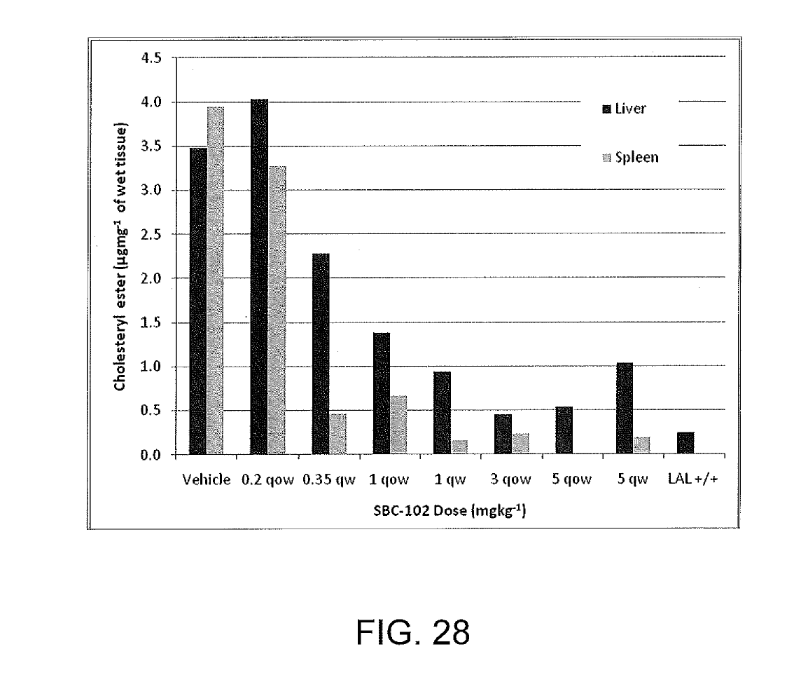

Based on the studies performed above, the pharmacodynamic (PD) effects of a range of doses and dose schedules (qw and qow) of LAL (“SBC-102”) were examined in LAL^{-/-} rats. In these studies, SBC-102 was administered by IV injections at dosages of 0.2, 1, 3 and 5 mg/kg, qow, or 0.35, 1 and 5 mg/kg, qw, for 1 month, beginning at 4 weeks of age. Results demonstrate improvements in body weight (BW) gain (FIG. 26), organomegaly (FIG. 27), and tissue substrate levels (FIG. 28). Serum transaminase levels were also reduced as the SBC-102 dose increased, with levels reaching essentially wild-type levels at the higher doses.

### Example 17

### Administration of Recombinant LAL in a Rat Model

The effects of repeat-dosing with recombinant human lysosomal acid lipase (LAL) on weight, tissue triglycerides and cholesterol, hepatomegaly, splenomegaly, lymphadenopathy, intestinal weight, and other parameters were evaluated in LAL Deficient Donryu rats described in Yoshida and Kuriyama (1990) Laboratory Animal Science, vol. 40, p 486-489 (see also Kuriyama et al. (1990) Journal of Lipid Research, vol. 31, p 1605-1611; Nakagawa et al., (1995) Journal of Lipid Research, vol. 36, p 2212-2218), the disclosure of which is incorporated in its entirety herein by reference.

At 4 weeks of age, Donryu rats homozygous for the LAL deletion (LAL were assigned into groups to either be dosed with recombinant human LAL produced in a transgenic chicken oviduct system or a saline placebo. Wild-type, age-matched, littermate rats were used as controls. The LAL^{-/-} rats were dosed once a week for four weeks (four doses total) or once every two weeks for four weeks (two doses total) by tail-vein injection as a single dose or in two equal doses given 30 minutes apart. Doses of LAL were 1 mg/kg or 5 mg/kg. Dosing schedule is shown in Table 4. The rats were pretreated with diphenhydramine (5 mg/kg) to counteract potential anaphylactic reactions, a procedure which is based on previous experiences in animal models of enzyme replacement therapy for the treatment of lysosomal storage disease (Shull et al. (1994) Proceedings of the National Academy of Science, vol. 91, p. 12937; Bielicki et al. (1999) The Journal of Biological Chemistry, 274, p. 36335; Vogler et al. (1999) Pediatric Research, 45, p. 838), the disclosure of which is incorporated in its entirety herein by reference.

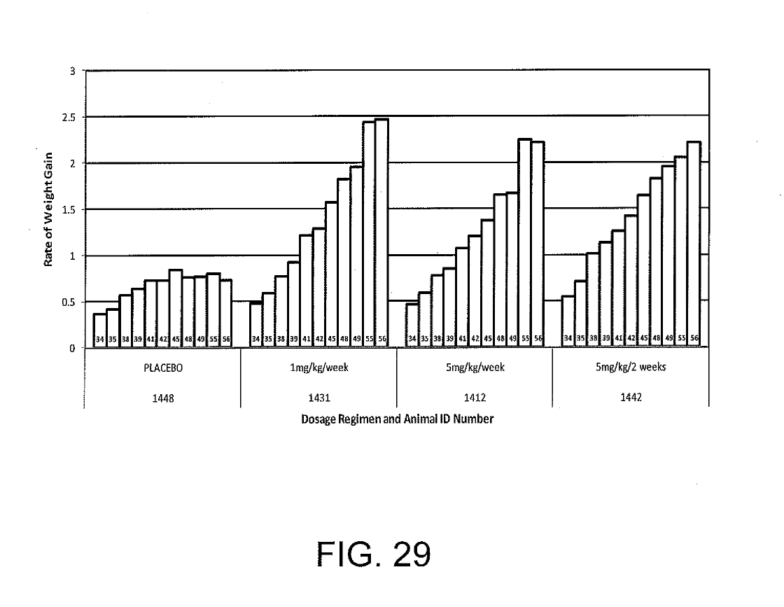

FIG. 29 shows the daily progress in weight gain of rats which were administered either 1 mg/kg of LAL per week or 5 mg/kg of LAL per week or 5 mg/kg of LAL per two weeks. It can be seen in the figure that there is little or no difference in therapeutic effect between the two dose sizes and frequencies.

TABLE 4
Weighing and Dosing Schedule

| Day from | | |
|---|---|---|
| Birth | Assessments/Injections Performed | |
| Day 13 | WEIGHED | |
| Day 14 | | |
| Day 20 | | |
| Day 21 | | Pups Weaned |
| Day 24 | | |
| Day 25 | | |
| Day 27 | | |
| Day 28 | | First Injection for administration once every |
| | | week and once every two weeks |
| Day 31 | | |
| Day 32 | | |
| Day 34 | | |
| Day 35 | | Second Injection for administration once every |
| | | week |
| Day 38 | | |
| Day 39 | | |
| Day 41 | | |
| Day 42 | | Third Injection for administration once every |
| | | week; Second administration for once every |
| | | two weeks |
| Day 45 | | |
| Day 48 | | |
| Day 49 | | Fourth injection for administration once every |
| | | week |
| Day 55 | | |
| Day 56 | | Necropsy |

Pathologic Examination of LAL^{-/-} Rats Treated with Recombinant LAL

At the termination of the study described in Example 1, study animals were humanely euthanized and necropsied to examine gross pathology, histopathology, and clinical chemistry. The gross necropsy included examination of the external surface of the body, all orifices, and the cranial, thoracic, and abdominal cavities and their contents. Mass of internal organs and tissues were determined for the rats and the organs and tissues were harvested and fixed in 10% neutral-buffered formalin. Following fixation, the tissues were processed and histological slides of hematoxylin and eosin-stained sections were prepared and evaluated.

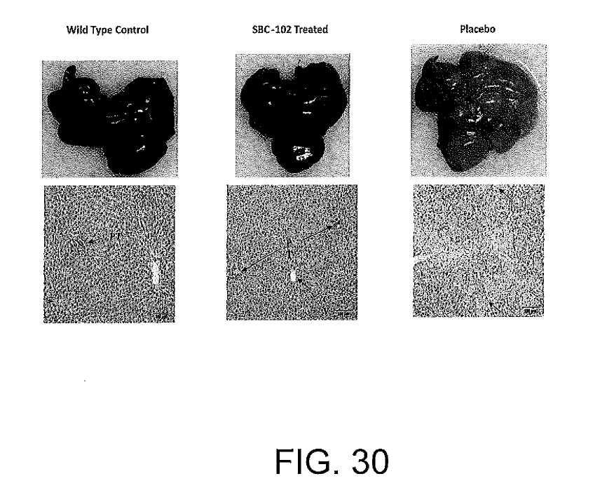

The gross pathological examination of treated animals analyzed showed a substantial normalization in liver size and color as can be seen in the dissection shown in FIG. 30. Organ-to-body weight ratios were determined and demonstrated a reduction in the relative organ size for liver, spleen, mesenteric tissue, duodenum, jejunum and ileum in successfully treated animals which were dissected, as compared to the placebo treated rats (FIG. 23). Histopathology of liver tissue from LAL of treated rats analyzed shows essentially normal liver histology in marked contrast to the substantial accumulation of foamy macrophages in the placebo-treated animals (FIG. 30).

### Example 18

### Treatment of Wolman Disease (WD) by Administration of Recombinant LAL

At 7 weeks of age a female patient is admitted to the hospital because of difficulty in weight gain and poor progress since birth. At the initial physical examination the patient weighs 3.6 kg (birth weight 3.7 kg) and is thin, with loose skin folds. The abdomen is distended, with firm hepatomegaly of 6 cm and firm splenomegaly of about 4 cm. Enlarged lymph nodes are noted in the groin and muscular activity is weak.

The initial hemoglobin level is 9.2 gm, platelets 506,000, and white blood cells 11,550. Urinalysis is normal, and bone marrow smears reveal vacuolated lymphocytes and numerous foam cells. Serum chemical measurements: total lipids 834 mg/100 ml, phospholipids 176 mg/100 ml, triglycerides 141 mg/100 ml, cholesterol 129 mg/100 ml, bilirubin 0.3 mg/100 ml, alkaline phosphatase 9.0 BU %, SGOT 90 units, SGPT 50 units, cholinesterase 20 units, urea nitrogen 8.3 mg, fasting sugar 45 mg/100 ml. CT scan of the abdomen shows hepatosplenomegaly and bilateral symmetrically enlarged adrenal glands with calcification.

The patient is surgically implanted with a venous vascular access port for dosing. After connecting the port to an ambulatory infusion machine, the patient is pretreated with 1 mg/kg of diphenhydramine 20 minutes prior to LAL infusion in order to counteract potential anaphylactic infusion reactions. The patient is then administered LAL at 1 mg/kg over the course of 5 hours by intravenous infusion. This therapy is repeated one time every 7 days indefinitely.

Within two weeks of administering the first dose of LAL, the patient experiences a significant improvement in weight gain and normalization in size of key abdominal organs as determined by ultrasound. Laboratory results demonstrate that infusion of the LAL restores lysosomal acid lipase activity in the patient and leads to correction of related abnormalities.

### Example 19

### Treatment of Cholesteryl Ester Storage Disease (CESD) by Administration of Recombinant LAL

A 3-year-old boy with a pruritic abdominal rash is examined by his pediatrician. Upon abdominal examination, hepatomegaly is noted by the physician and confirmed by ultrasound. At this point no diagnosis is made and the patient is monitored periodically.

At age 8, he is admitted to the hospital with gastroenteritis. Light microscopy of a liver biopsy shows increased intracytoplasmic glycogen and small lipid droplets in hepatocytes. Electron microscopy shows membrane-bound lipid droplets with small electron dense granules. A working diagnosis of glycogen storage disease type III (DeBrancher disease) is made, but skin fibroblast Debrancher activity is normal.

At age 10, hepatomegaly persists and a second liver biopsy is taken. Light microscopy shows altered lobular architecture of the hepatic parenchyma with distended hepatocytes containing cytoplasmic granules and vacuoles with mild periportal fibrosis. Fibroblast acid lipase activity is found to be 7% of normal, confirming the diagnosis of CESD. Plasma concentrations of total cholesterol (TC), triglycerides (TG), low-density lipoprotein cholesterol (LDL-C) are each above the 95th percentile for age and sex at 7.51, 3.24 and 5.58 mmol/L, respectively, while plasma high-density lipoprotein cholesterol (HDL-C) is below the 5th percentile at 0.47 mmol/L; he has combined hyperlipidemia (hypercholesterolemia, hypertriglyceridemia, hypoalphalipoproteinemia and hyperbetalipoproteinemia).

The patient is surgically implanted with a venous vascular access port for dosing. After connecting the port to an ambulatory infusion machine, the patient is pretreated with 5 mg/kg of diphenhydramine 20 minutes prior to LAL infusion in order to counteract potential anaphylactic infusion reactions. The patient is then administered LAL at 5 mg/kg over the course of 5 hours by intravenous infusion. This therapy is repeated one time every 14 days indefinitely.

Within two weeks of administering the first dose of LAL, the patient experiences a significant improvement in weight gain and normalization in size of key organs as determined by ultrasound. Laboratory results demonstrate that infusion of the LAL restores lysosomal acid lipase activity in the patient and leads to correction of related abnormalities.

### Example 20

### Description and Composition of the Medicinal Product

The drug substance of LAL described herein (“SBC-102”) is a recombinant human lysosomal acid lipase (rhLAL) purified from the egg white produced from transgenic Gallus. The excipients used in SBC-102 are similar to those used for other products for lysosomal storage disorders (LSD) currently on the market, and have been selected to maintain stability of the drug product.

SBC-102 is a clear, colorless, sterile liquid provided in a clear, Type I borosilicate glass vial with a non-natural latex (butyl), FluroTec®-coated stopper and aluminum crimp seal. SBC-102 is provided as an aqueous solution comprised of SBC-102 (2 mg/mL), Trisodium Citrate Dihydrate (13.7 mg/mL, USP), Citric Acid Monohydrate (1.57 mg/mL, USP), Human Serum Albumin (10 mg/mL, USP), and Water for Injection (to final volume, USP). The pH of SBC-102 is 5.9±0.2. SBC-102 contains no preservatives and vials are intended for single use only.

TABLE 5
Excipients in SBC-102 (LAL)

| Excipent | CAS number | Grade | Function |
|---|---|---|---|
| Trisodium Citrate | 6132-04-03 | USP | Buffer |
| Dihydrate | | | |
| Citric Acid Monohydrate | 5949-29-1 | USP | Buffer |
| Human Serum Albumin | 70024-90-7 | USP | Stabilizer |

### Components of the Drug Product

TABLE 6
Formulation of SBC-102

| Component | Concentration | |
|---|---|---|
| SBC-102 (rhLAL) | 2 | mg/mL* |
| Trisodium Citrate Dihydrate | 13.7 | mg/mL |
| Citric Acid Monohydrate | 1.57 | mg/mL |
| Human Serum Albumin | 10 | mg/mL |
| Water for Injection, QS to | 1.0 | mL |

### Example 21

### Characterization of Total Released N-Glycans

Characterization of the N-linked glycosylation present on SBC-102 was repeated by ultra-high pressure liquid chromatography (UPLC) with fluorescence and mass spectrometric detection with higher definition and sensitivity. Total release of the N-glycans was achieved through N-glycanase (PNGase F) endoglycosidase treatment on denatured SBC-102 for 24 hours.

Released N-glycans were labeled with 2-aminobenzamide (“2-AB”) for fluorescence detection through reductive amination. The glycans were dried followed by the addition of reducing agent, picolineborane and 2-AB. The reaction was incubated at 65° C. for 60-75 minutes. The labeled-glycans were purified from excess reagents and remaining protein using a hydrophilic membrane prior to analysis.

N-glycans were detected by fluorescence and mass detection using a quadrupole time-of-flight mass spectrometer. Identification of the glycans was confirmed by mass accuracy and relative retention time based upon a dextran-ladder calibration curve.

TABLE 7
N-glycoforms identified from total released analysis

| | | | Theoretical | Observed | |
|---|---|---|---|---|---|
| Peak | | Retention | MH+ | MH+ | % |
| # | Structure | time | (labeled) | (labeled) | Area |
| 1 | 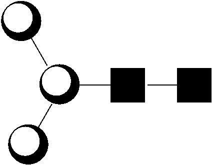 | 4.03 | 1031.40 | 1031.40 | 3.16 |
| 2 | 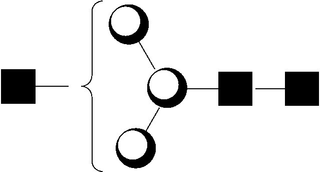 | 5.16 | 1234.48 | 1234.48 | 0.15 |
| 3 | 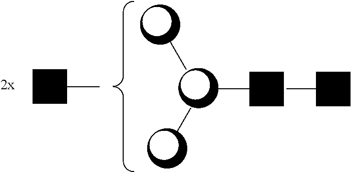 | 6.04 | 1437.56 | 1437.56 | 3.55 |
| 4 | 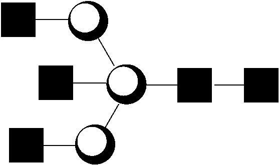 | 6.83 | 1604.64 | 1640.64 | 2.62 |
| 5 | 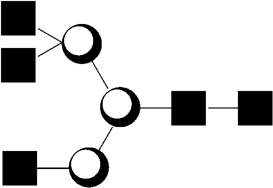 | 7.14 | 1640.64 | 1640.64 | 6.61 |
| 6 | 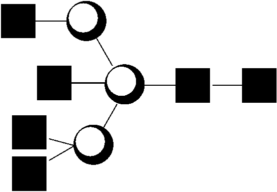 | 7.98 | 1843.72 | 1843.72 | 6.6 |
| 7 | 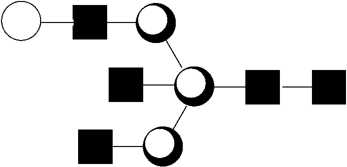 | 9.30 | 1802.69 | 1802.68 | 0.97 |
| 8 | 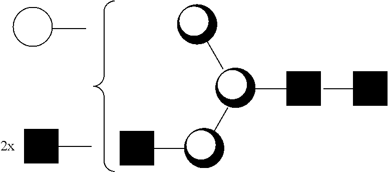 | 10.53 | 1802.69 | 1802.68 | 1.66 |
| 9 | 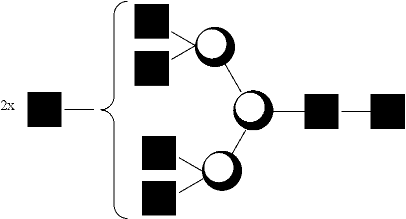 | 11.16 | 2249.88 | 2249.89 | 0.57 |
| 10 | 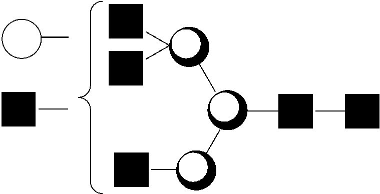 | 12.30 | 2005.77 | 2005.77 | 2.11 |
| 11 | 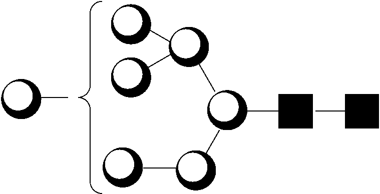 | 13.33 | 1679.62 | 1679.62 | 3.47 |
| 12 | 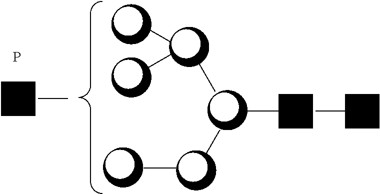 | 13.94 | 1800.61 | 1800.62 | 4.19 |
| 13 | 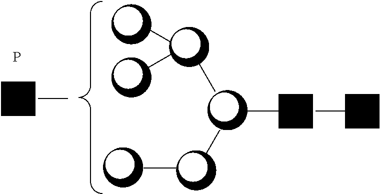 | 14.35 | 1800.61 | 1800.61 | 2.81 |
| 14 | 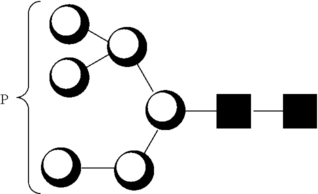 | 14.61 | 1597.54 | 1597.54 | 0.53 |
| 15 | 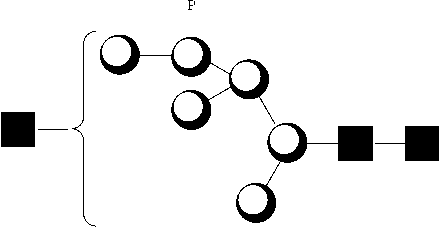 | 15.80 | 1800.61 | 1800.62 | 7.78 |
| 16 | 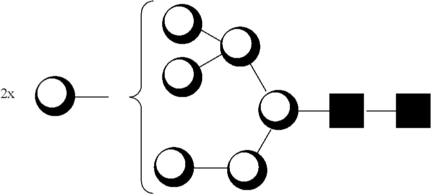 | 6.06 | 1841.67 | 1841.67 | 6.39 |
| 17 | 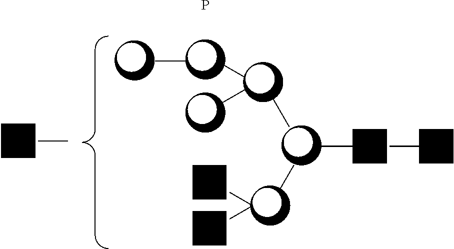 | 16.63 | 2206.77 | 2206.76 | 9.08 |
| 18 | 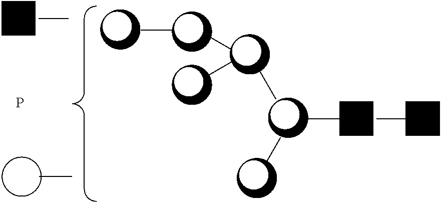 | 16.87 | 1962.66 | 1962.65 | 3.73 |
| 19 | 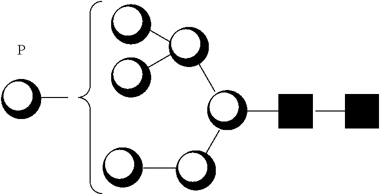 | 17.21 | 1759.58 | 1759.58 | 4.41 |
| 20 | 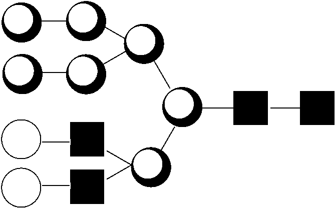 | 17.69 | 2409.88 | 2409.85 | 5.45 |
| 21 | 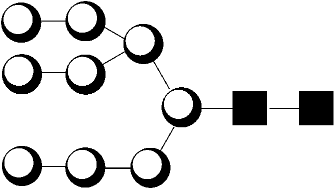 | 18.09 | 2003.72 | 2003.71 | 1.01 |
| 22 | 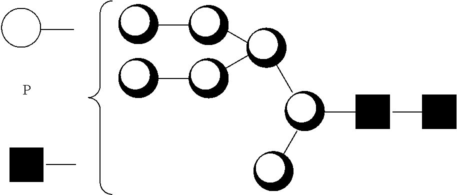 | 19.25 | 2124.71 | 2124.72 | 1 |
| 23 | 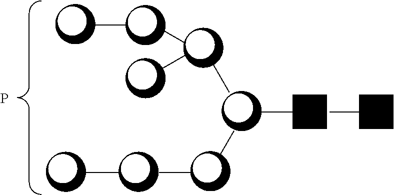 | 19.60 | 1921.63 | 1921.64 | 0.73 |
| 24 | 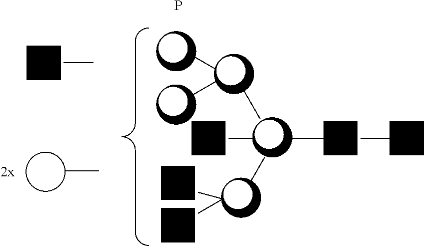 | 20.34 | 2571.90 | 2571.90 | 1.39 |
| 25 | 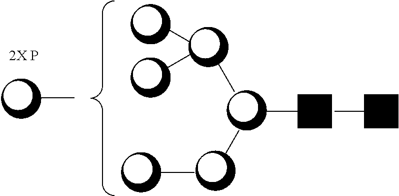 | 20.79 | 1839.55 | 1839.54 | 10.36 |
| 26 | 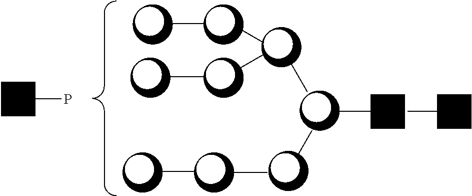 | 21.10 | 2286.76 | 2286.76 | 2.24 |
| 27 | 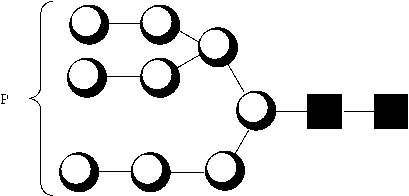 | 21.43 | 2083.69 | 2083.68 | 2.57 |

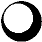

Mannose 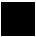
Glucosamine
◯ Hexose
P Phosphate

### Example 22

### Determination of Site-Specific Glycosylation

The type of N-glycans at each potential glycosylation site was determined by liquid chromatography-mass spectrometry (LC-MS) analysis of proteolytic digests. SBC-102 was denatured and cysteine alkylated followed by separate treatment with trypsin and endoproteinase AspN proteases. Separate proteolytic enzymes were required to produce SBC-102 glycopeptides of suitable size for LC-MS analysis. Separate matching samples were further treated with N-glycanase to generate the corresponding deglycosylated peptides.

The resulting tryptic and AspN glycopeptides/peptides were subjected to LC-MS and LC-MS/MS analysis. Glycopeptides were separated using a reversed-phase C18 UPLC column followed by mass detection on a quadrupole time-of-flight mass spectrometer. Glycopeptides were located and identified using MS/MS marker fragments, relative elution time to the deglycosylated peptide and known glycopeptide mass based upon glycoforms identified from the total released glycan characterization.

The additional glycans found in this analysis compared to previous analysis is a result of the orthogonal AspN sample preparation and the improved sensitivity of the LC-MS system. Specifically, the Asn⁷² (corresponding to Asn⁵¹ in SEQ ID NO:2) site where previously N-glycosylation was not identified in tryptic glycopeptides was identified in an AspN peptide. Additionally, ultra-high pressure chromatography and a state-of-the art electrospray time-of-flight mass spectrometry provide high sensitivity.

### Glycosylation at Asn⁷²

LAL protein contains 6 potential N-linked glycosylation sites. Namely, Asn³⁶, Asn⁷², Asn¹⁰¹ Asn¹⁶¹, Asn²⁷³ and Asn³²¹, which corresponds to positions Asn¹⁵, Asn⁵¹, Asn⁸⁰, Asn¹⁴⁰, Asn²⁵² and Asn³⁰⁰, respectively in the amino acid sequence set forth in SEQ ID NO:2.

Five of these N-glycosylation sites, Asn³⁶, Asn¹⁰¹, Asn¹⁶¹, Asn²⁷³ and Asn³²¹ were found to be consistently glycosylated as described in Table 2 and below. Asn⁷², however, could be either unglycosylated or glycosylated. In a mixture of LAL molecules, fewer Asn⁷² are glycosylated than any of Asn³⁶, Asn¹⁰¹, Asn¹⁶¹, Asn²⁷³ and Asn³²¹. Accordingly, one aspect of the invention is recombinant human LAL having a glycosylated Asn⁷² is within the scope of the invention.

Analyzed under the UPLC and mass spectrography for site-specific glycosylation, Asn⁷² was determined to contain glycoforms having one mannose-6-phosphate (M6P) or two M6Ps (bis-M6P), e.g., Phos1Man6GlcNAc2 and Phos2Man7GlcNAc2, which are shown below:

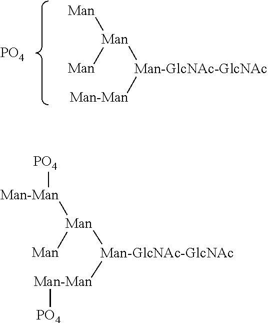

As shown above, the N-glycan structures at Asn⁷² primarily consist of a mixture of glycoforms structures with N-acetylglucosamine (GlcNAc2), mannose (Man) and mannose-6-phosphate (M6P or bis-M6P) as the major glycoforms.

### Glycosylation at Asn³⁶, Asn¹⁰¹ Asn¹⁶¹, Asn²⁷³ and Asn³²¹

Other glycoforms can be also associated with recombinant LAL protein at Asn³⁶, Asn¹⁰¹ Asn¹⁶¹, Asn²⁷³ and Asn³²¹ (corresponding to the positions Asn¹⁵, Asn⁸⁰, Asn¹⁴⁰, Asn²⁵² and Asn³⁰⁰, respectively, in the amino acid sequence set forth in SEQ ID NO:2). For example, the following glycoforms can be found at the indicated the asparagine residues.

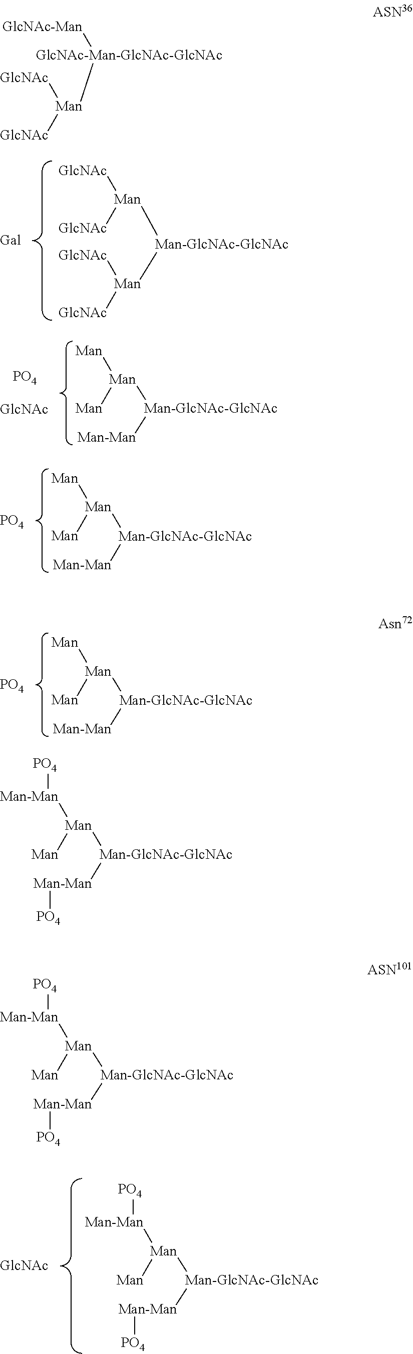

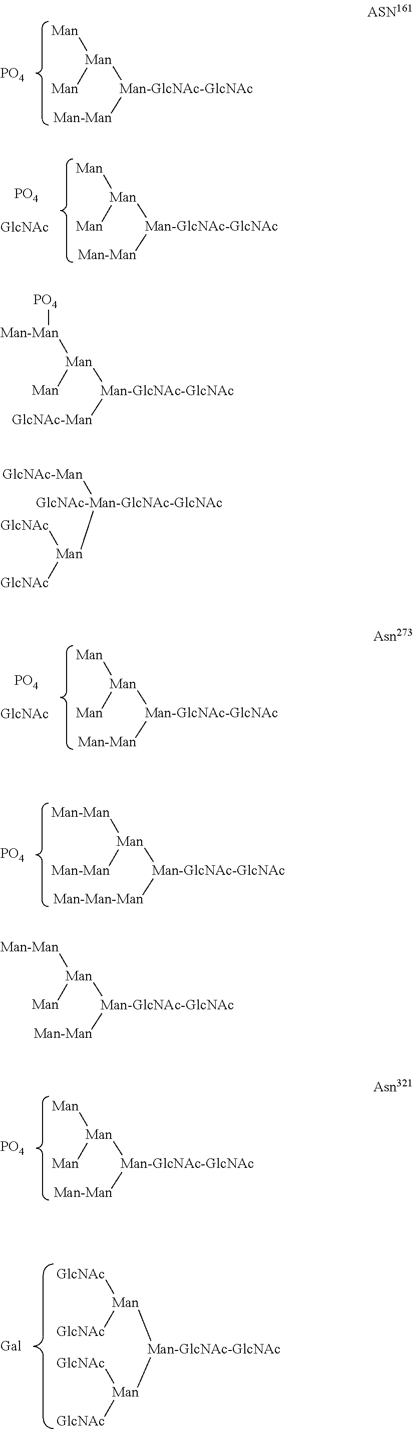

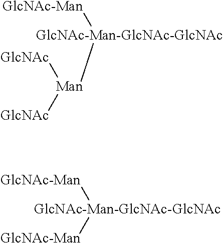

In addition, Table 8 provides a summary of monosaccharides species found in site-specific glycosylation analysis.

TABLE 8
Monosaccharide compositions from site-specific glycopeptide analysis

| | | | | Hexose |
|---|---|---|---|---|
| | GlcNAc | Man | Phos | (e.g., Galactose) |
| Asn³⁶ glycopeptide | 3.0 | 6.1 | 0.9 | 0.0 |
| Asn⁷² glycopeptide | 2.4 | 6.7 | 1.7 | 0.0 |
| Asn¹⁰¹ glycopeptide | 2.1 | 7.0 | 2.0 | 0.0 |
| Asn¹⁶¹ glycopeptide | 4.0 | 4.3 | 0.4 | 0.1 |
| Asn²⁷³ glycopeptide | 2.1 | 8.2 | 0.2 | 0.0 |
| Asn³²¹ glycopeptide | 4.8 | 3.6 | 0.2 | 0.1 |
| Total | 18.4 | 35.9 | 5.4 | 0.2 |

NOTE:
Monosaccharide mol/mol protein

Each example in the above specification is provided by way of explanation of the invention, not limitation of the invention. In fact, it will be apparent to those skilled in the art that various modifications, combinations, additions, deletions and variations can be made in the present invention without departing from the scope or spirit of the invention. For instance, features illustrated or described as part of one embodiment can be used in another embodiment to yield a still further embodiment. It is intended that the present invention cover such modifications, combinations, additions, deletions, and variations.

All documents (e.g., U.S. patents, U.S. patent applications, publications) cited in the above specification are incorporated herein by reference. Various modifications and variations of the present invention will be apparent to those skilled in the art without departing from the scope and spirit of the invention.

While this invention has been particularly shown and described with references to example embodiments thereof, it will be understood by those skilled in the art that various changes in form and details may be made therein without departing from the scope of the invention encompassed by the appended claims.

1-43. (canceled)

44. A composition comprising a mixture of recombinant human lysosomal acid lipase (LAL), the mixture comprising at least two human LAL selected from the group consisting of SEQ ID NO: 2, SEQ ID NO: 3, SEQ ID NO: 4 and SEQ ID NO:19.

45-68. (canceled)

69. A pharmaceutical formulation comprising a composition according to claim 44 in combination with a pharmaceutically acceptable carrier, diluent or excipient.

70. The pharmaceutical formulation of claim 69, wherein the formulation comprises at least one agent selected from the group consisting of trisodium citrate dehydrate, citric acid and human serum albumin.

71. The pharmaceutical formulation of claim 69, wherein the formulation is provided in an aqueous solution maintained at pH between about 5.6 and about 6.2.

72. The pharmaceutical formulation of claim 71, wherein the formulation is maintained at pH between 5.7 and 6.1.


SEQUENCE LISTING (20 sequences)

SEQ ID NO: 1 - protein, 399 aa, HOMO SAPIENS
MKMRFLGLVVCLVLWTLHSEGSGGKLTAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 2 - protein, 378 aa, HOMO SAPIENS
SGGKLTAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 3 - protein, 376 aa, HOMO SAPIENS
GKLTAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 4 - protein, 373 aa, HOMO SAPIENS
TAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 5 - DNA, 1150 bp, HOMO SAPIENS
atgaaaatgcggttcttggggttggtggtctgtttggttctctggaccctgcattccgaggggtccggagggaaactgacagctgtggatcctgaaacaaacatgaatgtcagtgaaattatctcttactggggattccctagtgaggaatacctagttgagacagaagatggatatattctgtgccttaaccgaattcctcatgggaggaagaaccattctgacaaaggtcccaaaccagttgtcttcctgcaacatggcttgctggcagattctagtaactgggtcacaaaccttgccaacagcagcctgggcttcattcttgctgatgctggttttgacgtgtggatgggcaacagcagaggaaatacctggtctcggaaacataagacactctcagtttctcaggatgaattctgggctttcagttatgatgagatggcaaaatatgacctaccagcttccattaacttcattctgaataagactggccaagaacaagtgtattatgtgggtcattctcaaggcaccactataggttttatagcattttcacagatccctgagctggctaaaaggattaaaatgttttttgccctgggtcctgtggcttccgtcgccttctgtactagccctatggccaaactgggacgactgccagatcatctcattaaggacctctttggagacaaagaatttcttccccagagtgcgtttttgaagtggctgggtacccacgtttgcactcatgtcatactgaaggagctctgtggaaatctctgttttcttctgtgtggatttaatgagagaaatttaaatatgtctagagtggatgtgtatacaacacattctcctgctggaacttctgtgcaaaacatgttacactggagccaggctgttaaattccaaaagtttcaagcctttgactggggaagcagtgccaagaattattttcattacaaccagagttatcctcccacatacaatgtgaaggacatgcttgtgccgactgcagtctggagcgggggtcacgactggcttgcagatgtctacgacgtcaatatcttactgactcagatcaccaacttggtgttccatgagagcattccggaatgggagcatcttgacttcatttggggcctggatgccc

SEQ ID NO: 6 - DNA, 10882 bp, HOMO SAPIENS
ctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgtattccctaacgatcacgtcggggtcaccaaatgaagccttctgcttcatgcatgtgctcgtagtcgtcagggaatcaacggtccggccatcaacccaggtgcacaccaatgtggtgaatggtcaaatggcgtttattgtatcgagctaggcacttaaatacaatatctctgcaatgcggaattcagtggttcgtccaatccgtccccctccctatgcaaaagcgaaactactatatcctgaggggactcctaaccgcgtacaaccgaagccccgcttttcgcctaaacatgctattgtcccctcagtcaagccttgcccgttacaacccgattcgcaagccttgccctccccacattatccgtagcattatttcctagcagtcatcagagctacagaagatactctatgctgtagccaagtctacaagtttactattcagcgacctcctatattccgcgtgccagccgatcaattaccaatccaaccagctatcacacggaatacaagaactcgcctacgctcttctttcgggctgcttataagcctcctgtaatttttttatattcctcgctcgagtctcttcagaatggcacagcaccgctgcagaaaaatgccaggtggactatgaactcacatccaaaggagcttgacctgatacctgattttcttcaaacaggggaaacaacacaatcccacaaaacagctcagagagaaaccatcactgatggctacagcaccaaggtatgcaatggcaatccattcgacattcatctgtgacctgagcaaaatgatttatctctccatgaatggttgcttctttccctcatgaaaaggcaatttccacactcacaatatgcaacaaagacaaacagagaacaattaatgtgctccttcctaatgttaaaattgtagtggcaaagaggagaacaaaatctcaagttctgagtaggttttagtgattggataagaggctttgacctgtgagctcacctggacttcatatccttttggataaaaagtgcttttataactttcaggtctccgagtctttattcatgagactgttggtttagggacagacccacaatgaaatgcctggcataggaaagggcagcagagccttagctgaccttttcttgggacaagcattgtcaaacaatgtgtgacaaaactatttgtactgctttgcacagctgtgctgggcagggcaatccattgccacctatcccaggtaaccttccaactgcaagaagattgttgcttactctctctagacccccaagtcaaaccaactatgcaggtatgctgacaacactatgatgacagcctgttctgatcaagatctcatttgttcatggacaatttttgttgcttgcagctggtcttccattgggaaagagtgtagtatatccttctcatctgacagaaaagcagaaattctcatgctccacacttaatctacattgttttaaaccaccggctacttcttggagaggaaaaatggcttttataagactcacaaaacaaagctctgcaagtcaaatgcatacaaaactgttctgtaggtctggaatcaggacactatgtggaagtcaaatagagcagctttaaaaagcctttgggatcattctcatcttatatttgcagcacgatactatgacagtgataactgacataactgcatcaatttccttgatattttatttgtcttaaagtacaagacatagagatggacgtaaagatggacatatgactcaggtctggacaggtccgtggtccatgtatgataaaagagatgaagggaaggagaattgagactgtctaagaagggcttcagggacgttctgaaggcagatttgactgaatcagatgtactgtccaagtctcatatgtagcaatggaaggctgatattggagaaatataaagaaatggctgtgaactcaaagtgaccctgaacagaaaagggatatggagttaaaataatgtcacagaactgaggtttatatgatataccatgggctgcagagggtcagagtgctccaccatgggcctctcttgggctgcagggaacttctgttctacacctggaacacctcctgccctcctccgcactgacctcagtgtcatcagggctgtttctctcacattttctcactcacctctcccaactaccattgtacagcagttgttcttacatattgctcctcctgaggtacatctagcatcgttaagtcctcagacttggcaaggagaatgtagatttccacagtatatatgttttcacaaaaggaaggagagaaacaaaagaaaatggcactgactaaacttcagctagtggtataggaaagtaattctgcttaacagagattgcagtgatctctatgtatgtcctgaagaattatgttgtacttttttcccccatttttaaatcaaacagtgctttacagaggtcagaatggtttctttactgtttgtcaattctattatttcaatacagaacaatagcttctataactgaaatatatttgctattgtatattatgattgtccctcgaaccatgaacactcctccagctgaatttcacaattcctctgtcatctgccaggccattaagttattcatggaagatctttgaggaacactgcaagttcatatcataaacacatttgaaattgagtattgttttgcattgtatggagctatgttttgctgtatcctcagaataaaagtttgttataaagcattcacacccataaaaagatagatttaaatattccaactataggaaagaaagtgtgtctgctcttcactctagtctcagttggctccttcacatgcacgcttctttatttctcctattttgtcaagaaaataataggtcaagtcttgttctcatttatgtcctgtctagcgtggctcagatgcacattgtacatacaagaaggatcaaatgaaacagacttctggtctgttactacaaccatagtaataagcacactaactaataattgctaattatgttttccatctccaaggttcccacatttttctgttttcttaaagatcccattatctggttgtaactgaagctcaatggaacatgagcaatatttcccagtcttctctcccatccaacagtcctgatggattagcagaacaggcagaaaacacattgttacccagaattaaaaactaatatttgctctccattcaatccaaaatggacctattgaaactaaaatctaacccaatcccattaaatgatttctatggtgtcaaaggtcaaacttctgaagggaacctgtgggtgggtcacaattcagactatatattccccagggctcagccagtgtctgtacatacagctagaaagctgtattgcctttagcagtcaagctcgaaaggtaagcaactctctggaattaccttctctctatattagctcttacttgcacctaaactttaaaaaattaacaattattgtgctatgtgttgtatctttaagggtgaagtacctgcgtgataccccctataaaaacttctcacctgtgtatgcattctgcactattttattatgtgtaaaagctttgtgtttgttttcaggaggcttattctttgtgcttaaaatatgtttttaatttcagaacatcttatcctgtcgttcactatctgatatgctttgcagtttgcttgattaacttctagccctacagagtgcacagagagcaaaatcatggtgttcagtgaattctggggagttattttaatgtgaaaattctctagaagtttaattcctgcaaagtgcagctgctgatcactacacaagataaaaatgtggggggtgcataaacgtatattcttacaataatagatacatgtgaacttatatacagaaaagaaaatgagaaaaatgtgtgtgtgtatactcacacacgtggtcagtaaaaacttttgaggggtttaatacagaaaatccaatcctgaggccccagcactcagtacgcatataaagggctgggctctgaaggacttctgactttcacagattatataaatctcaggaaagcaactagattcatgctggctccaaaagctgtgctttatataagcacactggctatacaatagttgtacagttcagctctttataatagaaacagacagaacaagtataaatcttctattggtctatgtcatgaacaagaattcattcagtggctctgttttatagtaaacattgctattttatcatgtctgcatttctcttctgtctgaatgtcaccactaaaatttaactccacagaaagtttatactacagtacacatgcatatctttgagcaaagcaaaccatacctgaaagtgcaatagagcagaatatgaattacatgcgtgtctttctcctagactacatgaccccatataaattacattccttatctattctgccatcaccaaaacaaaggtaaaaatacttttgaagatctactcatagcaagtagtgtgcaacaaacagatatttctctacatttatttttagggaataaaaataagaaataaaatagtcagcaagcctctgctttctcatatatctgtccaaacctaaagtttactgaaatttgctctttgaatttccagttttgcaagcctatcagattgtgttttaatcagaggtactgaaaagtatcaatgaattctagctttcactgaacaaaaatatgtagaggcaactggcttctgggacagtttgctacccaaaagacaactgaatgcaaatacataaatagatttatgaatatggttttgaacatgcacatgagaggtggatatagcaacagacacattaccacagaattactttaaaactacttgttaacatttaattgcctaaaaactgctcgtaatttactgttgtagcctaccatagagtaccctgcatggtactatgtacagcattccatccttacattttcactgttctgctgtttgctctagacaactcagagttcaccatgaaaatgcggttcttggggttggtggtctgtttggttctctggaccctgcattccgaggggtccggagggaaactgacagctgtggatccagaaacaaacatgaatgtcagtgaaattatctcttactggggattccctagtgaggaatacctagttgagacagaagatggatatattctgtgccttaaccgaattcctcatgggaggaagaaccattctgacaaaggtcccaaaccagttgtcttcctgcaacatggcttgctggcagattctagtaactgggtcacaaaccttgccaacagcagcctgggcttcattcttgctgatgctggttttgacgtgtggatgggcaacagcagaggaaatacctggtctcggaaacataagacactctcagtttctcaggatgaattctgggctttcagttatgatgagatggcaaaatatgacctaccagcttccattaacttcattctgaataagactggccaagaacaagtgtattatgtgggtcattctcaaggcaccactataggttttatagcattttcacagatccctgagctggctaaaaggattaaaatgttttttgccctgggtcctgtggcttccgtcgccttctgtactagccctatggccaaactgggacgactgccagatcatctcattaaggacctctttggagacaaagaatttcttccccagagtgcgtttttgaagtggctgggtacccacgtttgcactcatgtcatactgaaggagctctgtggaaatctctgttttcttctgtgtggatttaatgagagaaatttaaatatgtctagagtggatgtgtatacaacacattctcctgctggaacttctgtgcaaaacatgttacactggagccaggctgttaaattccaaaagtttcaagcctttgactggggaagcagtgccaagaattattttcattacaaccagagttatcctcccacatacaatgtgaaggacatgcttgtgccgactgcagtctggagcgggggtcacgactggcttgcagatgtctacgacgtcaatatcttactgactcagatcaccaacttggtgttccatgagagcattccggaatgggagcatcttgacttcatttggggcctggatgccccttggaggctttataataagattattaatctaatgaggaaatatcagtgattcgaagcggccgcaagaagaaagctgaaaaactctgtcccttccaacaagacccagagcactgtagtatcaggggtaaaatgaaaagtatgttatctgctgcatccagacttcataaaagctggagcttaatctagaaaaaaaatcagaaagaaattacactgtgagaacaggtgcaattcacttttcctttacacagagtaatactggtaactcatggatgaaggcttaagggaatgaaattggactcacagtactgagtcatcacactgaaaaatgcaacctgatacatcagcagaaggtttatgggggaaaaatgcagccttccaattaagccagatatctgtatgaccaagctgctccagaattagtcactcaaaatctctcagattaaattatcaactgtcaccaaccattcctatgctgacaaggcaattgcttgttctctgtgttcctgatactacaaggctcttcctgacttcctaaagatgcattataaaaatcttataattcacatttctccctaaactttgactcaatcatggtatgttggcaaatatggtatattactattcaaattgttttccttgtacccatatgtaatgggtcttgtgaatgtgctcttttgttcctttaatcataataaaaacatgtttaagcaaacacttttcacttgtagtatttgaaggtaccggatctcgagccgccttcaatgcccccaaaaccaatccccaggtttttaactctcccgattttccaagtaccatagcccgctgagagagcgccgcggtaatgggatcccaggaccccggggaatataagtctgagggggacgtaagcaacccttccttttgtaacagggacaacatagcccctatttccttcttagaaggagaggttttcccgcaataggtcttacacgcggacgaaatcacctttatgacggcttccatgcttgatccaccgggcgaccggaatcacgcagagcaaccggaatcacgcctggggtggaccgctcagtcgtcgggcttccttcccgtcttccaacgactctctgagttctcggtagggtatgttggccccctgcagtagggctccctccgacgccactcagcttctgccctcctaagccgcagccccctctactagggtcatcgtccgctccccgaataagcgagacggatgaggacaggatcgccacgccgcctgtggccgaccactattccctaacgatcacgtcggggtcaccaaatgaagccttctgcttcatgcatgtgctcgtagtcgtcagggaatcaacggtccggccatcaacccaggtgcacaccaatgtggtgaatggtcaaatggcgtttattgtatcgagctaggcacttaaatacaatatctctgcaatgcggaattcagtggttcgtccaatccgtgttagacccgtctgttgccttcctaacaaggcacgatcataccacgatcataccaccttactcccaccaatcggcatgcacggtgctttttctctccttataaggcatgttgctaactcatcgttacataagcatgttgcaagactacaagagtattgcataagactacatttccccctccctatgcaaaagcgaaactactatatcctgaggggactcctaaccgcgtacaaccgaagccccgcttttcgcctaaacatgctattgtcccctcagtcaagccttgcccgttacaacccgattcgcaagccttgccctccccacattatccgtagcattatttcctagcagtcatcagagctacagaagatactctatgctgtagccaagtctacaagtttactattcagcgacctcctatattccgcgtgccagccgatcaattaccaatgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccgg

SEQ ID NO: 7 - DNA, 7780 bp, HOMO SAPIENS
aatgaagccttctgcttcatgcatgtgctcgtagtcgtcagggaatcaacggtccggccatcaacccaggtgcacaccaatgtggtgaatggtcaaatggcgtttattgtatcgagctaggcacttaaatacaatatctctgcaatgcggaattcagtggttcgtccaatccgtccccctccctatgcaaaagcgaaactactatatcctgaggggactcctaaccgcgtacaaccgaagccccgcttttcgcctaaacatgctattgtcccctcagtcaagccttgcccgttacaacccgattcgcaagccttgccctccccacattatccgtagcattatttcctagcagtcatcagagctacagaagatactctatgctgtagccaagtctacaagtttactattcagcgacctcctatattccgcgtgccagccgatcaattaccaatccaaccagctatcacacggaatacaagaactcgcctacgctcttctttcgggctgcttataagcctcctgtaatttttttatattcctcgctcgagtctcttcagaatggcacagcaccgctgcagaaaaatgccaggtggactatgaactcacatccaaaggagcttgacctgatacctgattttcttcaaacaggggaaacaacacaatcccacaaaacagctcagagagaaaccatcactgatggctacagcaccaaggtatgcaatggcaatccattcgacattcatctgtgacctgagcaaaatgatttatctctccatgaatggttgcttctttccctcatgaaaaggcaatttccacactcacaatatgcaacaaagacaaacagagaacaattaatgtgctccttcctaatgttaaaattgtagtggcaaagaggagaacaaaatctcaagttctgagtaggttttagtgattggataagaggctttgacctgtgagctcacctggacttcatatccttttggataaaaagtgcttttataactttcaggtctccgagtctttattcatgagactgttggtttagggacagacccacaatgaaatgcctggcataggaaagggcagcagagccttagctgaccttttcttgggacaagcattgtcaaacaatgtgtgacaaaactatttgtactgctttgcacagctgtgctgggcagggcaatccattgccacctatcccaggtaaccttccaactgcaagaagattgttgcttactctctctagacccccaagtcaaaccaactatgcaggtatgctgacaacactatgatgacagcctgttctgatcaagatctcatttgttcatggacaatttttgttgcttgcagctggtcttccattgggaaagagtgtagtatatccttctcatctgacagaaaagcagaaattctcatgctccacacttaatctacattgttttaaaccaccggctacttcttggagaggaaaaatggcttttataagactcacaaaacaaagctctgcaagtcaaatgcatacaaaactgttctgtaggtctggaatcaggacactatgtggaagtcaaatagagcagctttaaaaagcctttgggatcattctcatcttatatttgcagcacgatactatgacagtgataactgacataactgcatcaatttccttgatattttatttgtcttaaagtacaagacatagagatggacgtaaagatggacatatgactcaggtctggacaggtccgtggtccatgtatgataaaagagatgaagggaaggagaattgagactgtctaagaagggcttcagggacgttctgaaggcagatttgactgaatcagatgtactgtccaagtctcatatgtagcaatggaaggctgatattggagaaatataaagaaatggctgtgaactcaaagtgaccctgaacagaaaagggatatggagttaaaataatgtcacagaactgaggtttatatgatataccatgggctgcagagggtcagagtgctccaccatgggcctctcttgggctgcagggaacttctgttctacacctggaacacctcctgccctcctccgcactgacctcagtgtcatcagggctgtttctctcacattttctcactcacctctcccaactaccattgtacagcagttgttcttacatattgctcctcctgaggtacatctagcatcgttaagtcctcagacttggcaaggagaatgtagatttccacagtatatatgttttcacaaaaggaaggagagaaacaaaagaaaatggcactgactaaacttcagctagtggtataggaaagtaattctgcttaacagagattgcagtgatctctatgtatgtcctgaagaattatgttgtacttttttcccccatttttaaatcaaacagtgctttacagaggtcagaatggtttctttactgtttgtcaattctattatttcaatacagaacaatagcttctataactgaaatatatttgctattgtatattatgattgtccctcgaaccatgaacactcctccagctgaatttcacaattcctctgtcatctgccaggccattaagttattcatggaagatctttgaggaacactgcaagttcatatcataaacacatttgaaattgagtattgttttgcattgtatggagctatgttttgctgtatcctcagaataaaagtttgttataaagcattcacacccataaaaagatagatttaaatattccaactataggaaagaaagtgtgtctgctcttcactctagtctcagttggctccttcacatgcacgcttctttatttctcctattttgtcaagaaaataataggtcaagtcttgttctcatttatgtcctgtctagcgtggctcagatgcacattgtacatacaagaaggatcaaatgaaacagacttctggtctgttactacaaccatagtaataagcacactaactaataattgctaattatgttttccatctccaaggttcccacatttttctgttttcttaaagatcccattatctggttgtaactgaagctcaatggaacatgagcaatatttcccagtcttctctcccatccaacagtcctgatggattagcagaacaggcagaaaacacattgttacccagaattaaaaactaatatttgctctccattcaatccaaaatggacctattgaaactaaaatctaacccaatcccattaaatgatttctatggtgtcaaaggtcaaacttctgaagggaacctgtgggtgggtcacaattcagactatatattccccagggctcagccagtgtctgtacatacagctagaaagctgtattgcctttagcagtcaagctcgaaaggtaagcaactctctggaattaccttctctctatattagctcttacttgcacctaaactttaaaaaattaacaattattgtgctatgtgttgtatctttaagggtgaagtacctgcgtgataccccctataaaaacttctcacctgtgtatgcattctgcactattttattatgtgtaaaagctttgtgtttgttttcaggaggcttattctttgtgcttaaaatatgtttttaatttcagaacatcttatcctgtcgttcactatctgatatgctttgcagtttgcttgattaacttctagccctacagagtgcacagagagcaaaatcatggtgttcagtgaattctggggagttattttaatgtgaaaattctctagaagtttaattcctgcaaagtgcagctgctgatcactacacaagataaaaatgtggggggtgcataaacgtatattcttacaataatagatacatgtgaacttatatacagaaaagaaaatgagaaaaatgtgtgtgtgtatactcacacacgtggtcagtaaaaacttttgaggggtttaatacagaaaatccaatcctgaggccccagcactcagtacgcatataaagggctgggctctgaaggacttctgactttcacagattatataaatctcaggaaagcaactagattcatgctggctccaaaagctgtgctttatataagcacactggctatacaatagttgtacagttcagctctttataatagaaacagacagaacaagtataaatcttctattggtctatgtcatgaacaagaattcattcagtggctctgttttatagtaaacattgctattttatcatgtctgcatttctcttctgtctgaatgtcaccactaaaatttaactccacagaaagtttatactacagtacacatgcatatctttgagcaaagcaaaccatacctgaaagtgcaatagagcagaatatgaattacatgcgtgtctttctcctagactacatgaccccatataaattacattccttatctattctgccatcaccaaaacaaaggtaaaaatacttttgaagatctactcatagcaagtagtgtgcaacaaacagatatttctctacatttatttttagggaataaaaataagaaataaaatagtcagcaagcctctgctttctcatatatctgtccaaacctaaagtttactgaaatttgctctttgaatttccagttttgcaagcctatcagattgtgttttaatcagaggtactgaaaagtatcaatgaattctagctttcactgaacaaaaatatgtagaggcaactggcttctgggacagtttgctacccaaaagacaactgaatgcaaatacataaatagatttatgaatatggttttgaacatgcacatgagaggtggatatagcaacagacacattaccacagaattactttaaaactacttgttaacatttaattgcctaaaaactgctcgtaatttactgttgtagcctaccatagagtaccctgcatggtactatgtacagcattccatccttacattttcactgttctgctgtttgctctagacaactcagagttcaccatgaaaatgcggttcttggggttggtggtctgtttggttctctggaccctgcattccgaggggtccggagggaaactgacagctgtggatccagaaacaaacatgaatgtcagtgaaattatctcttactggggattccctagtgaggaatacctagttgagacagaagatggatatattctgtgccttaaccgaattcctcatgggaggaagaaccattctgacaaaggtcccaaaccagttgtcttcctgcaacatggcttgctggcagattctagtaactgggtcacaaaccttgccaacagcagcctgggcttcattcttgctgatgctggttttgacgtgtggatgggcaacagcagaggaaatacctggtctcggaaacataagacactctcagtttctcaggatgaattctgggctttcagttatgatgagatggcaaaatatgacctaccagcttccattaacttcattctgaataagactggccaagaacaagtgtattatgtgggtcattctcaaggcaccactataggttttatagcattttcacagatccctgagctggctaaaaggattaaaatgttttttgccctgggtcctgtggcttccgtcgccttctgtactagccctatggccaaactgggacgactgccagatcatctcattaaggacctctttggagacaaagaatttcttccccagagtgcgtttttgaagtggctgggtacccacgtttgcactcatgtcatactgaaggagctctgtggaaatctctgttttcttctgtgtggatttaatgagagaaatttaaatatgtctagagtggatgtgtatacaacacattctcctgctggaacttctgtgcaaaacatgttacactggagccaggctgttaaattccaaaagtttcaagcctttgactggggaagcagtgccaagaattattttcattacaaccagagttatcctcccacatacaatgtgaaggacatgcttgtgccgactgcagtctggagcgggggtcacgactggcttgcagatgtctacgacgtcaatatcttactgactcagatcaccaacttggtgttccatgagagcattccggaatgggagcatcttgacttcatttggggcctggatgccccttggaggctttataataagattattaatctaatgaggaaatatcagtgattcgaagcggccgcaagaagaaagctgaaaaactctgtcccttccaacaagacccagagcactgtagtatcaggggtaaaatgaaaagtatgttatctgctgcatccagacttcataaaagctggagcttaatctagaaaaaaaatcagaaagaaattacactgtgagaacaggtgcaattcacttttcctttacacagagtaatactggtaactcatggatgaaggcttaagggaatgaaattggactcacagtactgagtcatcacactgaaaaatgcaacctgatacatcagcagaaggtttatgggggaaaaatgcagccttccaattaagccagatatctgtatgaccaagctgctccagaattagtcactcaaaatctctcagattaaattatcaactgtcaccaaccattcctatgctgacaaggcaattgcttgttctctgtgttcctgatactacaaggctcttcctgacttcctaaagatgcattataaaaatcttataattcacatttctccctaaactttgactcaatcatggtatgttggcaaatatggtatattactattcaaattgttttccttgtacccatatgtaatgggtcttgtgaatgtgctcttttgttcctttaatcataataaaaacatgtttaagcaaacacttttcacttgtagtatttgaaggtaccggatctcgagccgccttcaatgcccccaaaaccaatccccaggtttttaactctcccgattttccaagtaccatagcccgctgagagagcgccgcggtaatgggatcccaggaccccggggaatataagtctgagggggacgtaagcaacccttccttttgtaacagggacaacatagcccctatttccttcttagaaggagaggttttcccgcaataggtcttacacgcggacgaaatcacctttatgacggcttccatgcttgatccaccgggcgaccggaatcacgcagagcaaccggaatcacgcctggggtggaccgctcagtcgtcgggcttccttcccgtcttccaacgactctctgagttctcggtagggtatgttggccccctgcagtagggctccctccgacgccactcagcttctgccctcctaagccgcagccccctctactagggtcatcgtccgctccccgaataagcgagacggatgaggacaggatcgccacgccgcctgtggccgaccactattccctaacgatcacgtcggggtcaccaaatgaagccttctgcttcatgcatgtgctcgtagtcgtcagggaatcaacggtccggccatcaacccaggtgcacaccaatgtggtgaatggtcaaatggcgtttattgtatcgagctaggcacttaaatacaatatctctgcaatgcggaattcagtggttcgtccaatccgtgttagacccgtctgttgccttcctaacaaggcacgatcataccacgatcataccaccttactcccaccaatcggcatgcacggtgctttttctctccttataaggcatgttgctaactcatcgttacataagcatgttgcaagactacaagagtattgcataagactacatt

SEQ ID NO: 8 - DNA, 10762 bp, HOMO SAPIENS
  misc_feature (1)..(2735): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttcgaagcggccgcaagaagaaagctgaaaaactctgtcccttccaacaagacccagagcactgtagtatcaggggtaaaatgaaaagtatgttatctgctgcatccagacttcataaaagctggagcttaatctagaaaaaaaatcagaaagaaattacactgtgagaacaggtgcaattcacttttcctttacacagagtaatactggtaactcatggatgaaggcttaagggaatgaaattggactcacagtactgagtcatcacactgaaaaatgcaacctgatacatcagcagaaggtttatgggggaaaaatgcagccttccaattaagccagatatctgtatgaccaagctgctccagaattagtcactcaaaatctctcagattaaattatcaactgtcaccaaccattcctatgctgacaaggcaattgcttgttctctgtgttcctgatactacaaggctcttcctgacttcctaaagatgcattataaaaatcttataattcacatttctccctaaactttgactcaatcatggtatgttggcaaatatggtatattactattcaaattgttttccttgtacccatatgtaatgggtcttgtgaatgtgctcttttgttcctttaatcataataaaaacatgtttaagcaaacacttttcacttgtagtatttgaaggtaccggatctcgagccgccttcaatgcccccaaaaccaatccccaggtttttaactctcccgattttccaagtaccatagcccgctgagagagcgccgcggtaatgggatcccaggaccccggggaatataagtctgagggggacgtaagcaacccttccttttgtaacagggacaacatagcccctatttccttcttagaaggagaggttttcccgcaataggtcttacacgcggacgaaatcacctttatgacggcttccatgcttgatccaccgggcgaccggaatcacgcagagcaaccggaatcacgcctggggtggaccgctcagtcgtcgggcttccttcccgtcttccaacgactctctgagttctcggtagggtatgttggccccctgcagtagggctccctccgacgccactcagcttctgccctcctaagccgcagccccctctactagggtcatcgtccgctccccgaataagcgagacggatgaggacaggatcgccacgccgcctgtggccgaccactattccctaacgatcacgtcggggtcaccaaatgaagccttctgcttcatgcatgtgctcgtagtcgtcagggaatcaacggtccggccatcaacccaggtgcacaccaatgtggtgaatggtcaaatggcgtttattgtatcgagctaggcacttaaatacaatatctctgcaatgcggaattcagtggttcgtccaatccgtgttagacccgtctgttgccttcctaacaaggcacgatcataccacgatcataccaccttactcccaccaatcggcatgcacggtgctttttctctccttataaggcatgttgctaactcatcgttacataagcatgttgcaagactacaagagtattgcataagactacatttccccctccctatgcaaaagcgaaactactatatcctgaggggactcctaaccgcgtacaaccgaagccccgcttttcgcctaaacatgctattgtcccctcagtcaagccttgcccgttacaacccgattcgcaagccttgccctccccacattatccgtagcattatttcctagcagtcatcagagctacagaagatactctatgctgtagccaagtctacaagtttactattcagcgacctcctatattccgcgtgccagccgatcaattaccaatgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgtattccctaacgatcacgtcggggtcaccaaatgaagccttctgcttcatgcatgtgctcgtagtcgtcagggaatcaacggtccggccatcaacccaggtgcacaccaatgtggtgaatggtcaaatggcgtttattgtatcgagctaggcacttaaatacaatatctctgcaatgcggaattcagtggttcgtccaatccgtccccctccctatgcaaaagcgaaactactatatcctgaggggactcctaaccgcgtacaaccgaagccccgcttttcgcctaaacatgctattgtcccctcagtcaagccttgcccgttacaacccgattcgcaagccttgccctccccacattatccgtagcattatttcctagcagtcatcagagctacagaagatactctatgctgtagccaagtctacaagtttactattcagcgacctcctatattccgcgtgccagccgatcaattaccaatccaaccagctatcacacggaatacaagaactcgcctacgctcttctttcgggctgcttataagcctcctgtaatttttttatattcctcgttaagtcctcagacttggcaaggagaatgtagatttccacagtatatatgttttcacaaaaggaaggagagaaacaaaagaaaatggcactgactaaacttcagctagtggtataggaaagtaattctgcttaacagagattgcagtgatctctatgtatgtcctgaagaattatgttgtacttttttcccccatttttaaatcaaacagtgctttacagaggtcagaatggtttctttactgtttgtcaattctattatttcaatacagaacaatagcttctataactgaaatatatttgctattgtatattatgattgtccctcgaaccatgaacactcctccagctgaatttcacaattcctctgtcatctgccaggccattaagttattcatggaagatctttgaggaacactgcaagttcatatcataaacacatttgaaattgagtattgttttgcattgtatggagctatgttttgctgtatcctcagaataaaagtttgttataaagcattcacacccataaaaagatagatttaaatattccaactataggaaagaaagtgtgtctgctcttcactctagtctcagttggctccttcacatgcacgcttctttatttctcctattttgtcaagaaaataataggtcaagtcttgttctcatttatgtcctgtctagcgtggctcagatgcacattgtacatacaagaaggatcaaatgaaacagacttctggtctgttactacaaccatagtaataagcacactaactaataattgctaattatgttttccatctccaaggttcccacatttttctgttttcttaaagatcccattatctggttgtaactgaagctcaatggaacatgagcaatatttcccagtcttctctcccatccaacagtcctgatggattagcagaacaggcagaaaacacattgttacccagaattaaaaactaatatttgctctccattcaatccaaaatggacctattgaaactaaaatctaacccaatcccattaaatgatttctatggtgtcaaaggtcaaacttctgaagggaacctgtgggtgggtcacaattcagactatatattccccagggctcagccagtgtctgtacatacagctagaaagctgtattgcctttagcagtcaagctcgaaaggtaagcaactctctggaattaccttctctctatattagctcttacttgcacctaaactttaaaaaattaacaattattgtgctatgtgttgtatctttaagggtgaagtacctgcgtgataccccctataaaaacttctcacctgtgtatgcattctgcactattttattatgtgtaaaagctttgtgtttgttttcaggaggcttattctttgtgcttaaaatatgtttttaatttcagaacatcttatcctgtcgttcactatctgatatgctttgcagtttgcttgattaacttctagccctacagagtgcacagagagcaaaatcatggtgttcagtgaattctggggagttattttaatgtgaaaattctctagaagtttaattcctgcaaagtgcagctgctgatcactacacaagataaaaatgtggggggtgcataaacgtatattcttacaataatagatacatgtgaacttatatacagaaaagaaaatgagaaaaatgtgtgtgtgtatactcacacacgtggtcagtaaaaacttttgaggggtttaatacagaaaatccaatcctgaggccccagcactcagtacgcatataaagggctgggctctgaaggacttctgactttcacagattatataaatctcaggaaagcaactagattcatgctggctccaaaagctgtgctttatataagcacactggctatacaatagttgtacagttcagctctttataatagaaacagacagaacaagtataaatcttctattggtctatgtcatgaacaagaattcattcagtggctctgttttatagtaaacattgctattttatcatgtctgcatttctcttctgtctgaatgtcaccactaaaatttaactccacagaaagtttatactacagtacacatgcatatctttgagcaaagcaaaccatacctgaaagtgcaatagagcagaatatgaattacatgcgtgtctttctcctagactacatgaccccatataaattacattccttatctattctgccatcaccaaaacaaaggtaaaaatacttttgaagatctactcatagcaagtagtgtgcaacaaacagatatttctctacatttatttttagggaataaaaataagaaataaaatagtcagcaagcctctgctttctcatatatctgtccaaacctaaagtttactgaaatttgctctttgaatttccagttttgcaagcctatcagattgtgttttaatcagaggtactgaaaagtatcaatgaattctagctttcactgaacaaaaatatgtagaggcaactggcttctgggacagtttgctacccaaaagacaactgaatgcaaatacataaatagatttatgaatatggttttgaacatgcacatgagaggtggatatagcaacagacacattaccacagaattactttaaaactacttgttaacatttaattgcctaaaaactgctcgtaatttactgttgtagcctaccatagagtaccctgcatggtactatgtacagcattccatccttacattttcactgttctgctgtttgctctagacaactcagagttcaccatg

SEQ ID NO: 9 - DNA, 242 bp, HOMO SAPIENS
cccgggttgttaacatttaattgcctaaaaactgctcgtaatttactgttgtagcctaccatagagtaccctgcatggtactatgtacagcattccatccttacattttcactgttctgctgtttgctctagacaactcagagttcaccatgaaaatgcggttcttggggttggtggtctgtttggttctctggaccctgcattccgaggggtccggagggaaactgacagctgtggatcct

SEQ ID NO: 10 - DNA, 1575 bp, HOMO SAPIENS
ccattatctggttgtaactgaagctcaatggaacatgagcaatatttcccagtcttctctcccatccaacagtcctgatggattagcagaacaggcagaaaacacattgttacccagaattaaaaactaatatttgctctccattcaatccaaaatggacctattgaaactaaaatctaacccaatcccattaaatgatttctatggtgtcaaaggtcaaacttctgaagggaacctgtgggtgggtcacaattcagactatatattccccagggctcagccagtgtctgtacatacagctagaaagctgtattgcctttagcagtcaagctcgaaagacaactcagagttcaccatgaaaatgcggttcttggggttggtggtctgtttggttctctggaccctgcattccgaggggtccggagggaaactgacagctgtggatcctgaaacaaacatgaatgtcagtgaaattatctcttactggggattccctagtgaggaatacctagttgagacagaagatggatatattctgtgccttaaccgaattcctcatgggaggaagaaccattctgacaaaggtcccaaaccagttgtcttcctgcaacatggcttgctggcagattctagtaactgggtcacaaaccttgccaacagcagcctgggcttcattcttgctgatgctggttttgacgtgtggatgggcaacagcagaggaaatacctggtctcggaaacataagacactctcagtttctcaggatgaattctgggctttcagttatgatgagatggcaaaatatgacctaccagcttccattaacttcattctgaataagactggccaagaacaagtgtattatgtgggtcattctcaaggcaccactataggttttatagcattttcacagatccctgagctggctaaaaggattaaaatgttttttgccctgggtcctgtggcttccgtcgccttctgtactagccctatggccaaactgggacgactgccagatcatctcattaaggacctctttggagacaaagaatttcttccccagagtgcgtttttgaagtggctgggtacccacgtttgcactcatgtcatactgaaggagctctgtggaaatctctgttttcttctgtgtggatttaatgagagaaatttaaatatgtctagagtggatgtgtatacaacacattctcctgctggaacttctgtgcaaaacatgttacactggagccaggctgttaaattccaaaagtttcaagcctttgactggggaagcagtgccaagaattattttcattacaaccagagttatcctcccacatacaatgtgaaggacatgcttgtgccgactgcagtctggagcgggggtcacgactggcttgcagatgtctacgacgtcaatatcttactgactcagatcaccaacttggtgttccatgagagcattccggaatgggagcatcttgacttcatttggggcctggatgccccttggaggctttataataagattattaatctaatgaggaaatatcagtgattcgaagcggccgccccggg

SEQ ID NO: 11 - DNA, 2789 bp, HOMO SAPIENS
ctcgagtctcttcagaatggcacagcaccgctgcagaaaaatgccaggtggactatgaactcacatccaaaggagcttgacctgatacctgattttcttcaaacaggggaaacaacacaatcccacaaaacagctcagagagaaaccatcactgatggctacagcaccaaggtatgcaatggcaatccattcgacattcatctgtgacctgagcaaaatgatttatctctccatgaatggttgcttctttccctcatgaaaaggcaatttccacactcacaatatgcaacaaagacaaacagagaacaattaatgtgctccttcctaatgttaaaattgtagtggcaaagaggagaacaaaatctcaagttctgagtaggttttagtgattggataagaggctttgacctgtgagctcacctggacttcatatccttttggataaaaagtgcttttataactttcaggtctccgagtctttattcatgagactgttggtttagggacagacccacaatgaaatgcctggcataggaaagggcagcagagccttagctgaccttttcttgggacaagcattgtcaaacaatgtgtgacaaaactatttgtactgctttgcacagctgtgctgggcagggcaatccattgccacctatcccaggtaaccttccaactgcaagaagattgttgcttactctctctagacccccaagtcaaaccaactatgcaggtatgctgacaacactatgatgacagcctgttctgatcaagatctcatttgttcatggacaatttttgttgcttgcagctggtcttccattgggaaagagtgtagtatatccttctcatctgacagaaaagcagaaattctcatgctccacacttaatctacattgttttaaaccaccggctacttcttggagaggaaaaatggcttttataagactcacaaaacaaagctctgcaagtcaaatgcatacaaaactgttctgtaggtctggaatcaggacactatgtggaagtcaaatagagcagctttaaaaagcctttgggatcattctcatcttatatttgcagcacgatactatgacagtgataactgacataactgcatcaatttccttgatattttatttgtcttaaagtacaagacatagagatggacgtaaagatggacatatgactcaggtctggacaggtccgtggtccatgtatgataaaagagatgaagggaaggagaattgagactgtctaagaagggcttcagggacgttctgaaggcagatttgactgaatcagatgtactgtccaagtctcatatgtagcaatggaaggctgatattggagaaatataaagaaatggctgtgaactcaaagtgaccctgaacagaaaagggatatggagttaaaataatgtcacagaactgaggtttatatgatataccatgggctgcagagggtcagagtgctccaccatgggcctctcttgggctgcagggaacttctgttctacacctggaacacctcctgccctcctccgcactgacctcagtgtcatcagggctgtttctctcacattttctcactcacctctcccaactaccattgtacagcagttgttcttacatattgctcctcctgaggtacatctagcatcgttaagtcctcagacttggcaaggagaatgtagatttccacagtatatatgttttcacaaaaggaaggagagaaacaaaagaaaatggcactgactaaacttcagctagtggtataggaaagtaattctgcttaacagagattgcagtgatctctatgtatgtcctgaagaattatgttgtacttttttcccccatttttaaatcaaacagtgctttacagaggtcagaatggtttctttactgtttgtcaattctattatttcaatacagaacaatagcttctataactgaaatatatttgctattgtatattatgattgtccctcgaaccatgaacactcctccagctgaatttcacaattcctctgtcatctgccaggccattaagttattcatggaagatctttgaggaacactgcaagttcatatcataaacacatttgaaattgagtattgttttgcattgtatggagctatgttttgctgtatcctcagaataaaagtttgttataaagcattcacacccataaaaagatagatttaaatattccaactataggaaagaaagtgtgtctgctcttcactctagtctcagttggctccttcacatgcacgcttctttatttctcctattttgtcaagaaaataataggtcaagtcttgttctcatttatgtcctgtctagcgtggctcagatgcacattgtacatacaagaaggatcaaatgaaacagacttctggtctgttactacaaccatagtaataagcacactaactaataattgctaattatgttttccatctccaaggttcccacatttttctgttttcttaaagatcccattatctggttgtaactgaagctcaatggaacatgagcaatatttcccagtcttctctcccatccaacagtcctgatggattagcagaacaggcagaaaacacattgttacccagaattaaaaactaatatttgctctccattcaatccaaaatggacctattgaaactaaaatctaacccaatcccattaaatgatttctatggtgtcaaaggtcaaacttctgaagggaacctgtgggtgggtcacaattcagactatatattccccagggctcagccagtgtctgt

SEQ ID NO: 12 - DNA, 20 bp, HOMO SAPIENS
agaaactgagagtgtcttat

SEQ ID NO: 13 - DNA, 29 bp, HOMO SAPIENS
tgacagctgtggatccagaaacaaacatg

SEQ ID NO: 14 - DNA, 29 bp, HOMO SAPIENS
gccgctcgagcgaggaatataaaaaaatt

SEQ ID NO: 15 - DNA, 20 bp, HOMO SAPIENS
tccgcgcacatttccccgaa

SEQ ID NO: 16 - DNA, 21 bp, HOMO SAPIENS
acgactggcttgcagatgtct

SEQ ID NO: 17 - DNA, 22 bp, HOMO SAPIENS
ccccaaatgaagtcaagatgct

SEQ ID NO: 18 - DNA, 25 bp, HOMO SAPIENS
ccggaatgctctcatggaacaccaa

SEQ ID NO: 19 - protein, 372 aa, HOMO SAPIENS
AVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 20 - protein, 399 aa, HOMO SAPIENS; identical to SEQ ID NO: 1